(19) United States
(12) Patent Application Publication
(10) Pub. No.: US 2024/0076620 A1
(43) Pub. Date: Mar. 7, 2024

(54) PRODUCTION OF SKELETAL MUSCLE CELLS AND SKELETAL MUSCLE TISSUE FROM PLURIPOTENT STEM CELLS

(71) Applicant: Georg-August-Universität Göttingen Stiftung Öffentlichen Rechts, Universitätsmedizin, Göttingen (DE)

(72) Inventors: Wolfram-Hubertus ZIMMERMANN, Göttingen (DE); Malte TIBURCY, Göttingen (DE); Mina SHAHRIYARI, Göttingen (DE)

(73) Assignee: Georg-August-Universität Göttingen Stiftung Öffentlichen Rechts, Universitätsmedizin, Göttingen (DE)

(21) Appl. No.: 17/768,520

(22) PCT Filed: Oct. 13, 2020
(86) PCT No.: PCT/EP2020/078738

§ 371 Date: Apr. 13, 2022

(30) Foreign Application Priority Data
Oct. 14, 2019 (DE) 10 2019 127 604.7

(51) Int. Cl. C12N 5/077; A61K 35/34; C12N 5/00; G01N 33/50

(52) U.S. Cl. CPC C12N 5/0659; A61K 35/34; C12N 5/0018; G01N 33/5023; C12N 2500/25; C12N 2500/46; C12N 2501/115; C12N 2501/12; C12N 2501/392; C12N 2501/42; C12N 2501/998; C12N 2501/999

(57) ABSTRACT

The application describes methods for producing artificial skeletal muscle tissue from pluripotent stem cells. A method for producing skeletal myoblasts, skeletal myotubes and satellite cells from pluripotent stem cells is also disclosed. During the described methods, there is directed differentiation and maturation of the pluripotent stem cells into skeletal myotubes and satellite cells. The application also describes artificial skeletal muscle tissue which has multinuclear skeletal muscle fibres with satellite cells. Furthermore, the invention relates to mesodermally differentiated skeletal myoblast precursor cells, myogenically specified skeletal myoblast precursor cells, skeletal myoblast cells, satellite cells and skeletal myotubes, which can be produced by means of the disclosed methods. The application also describes the use of skeletal muscle tissue or the disclosed cells in drug testing or in medicine. Lastly, the application relates to in vitro methods in which the skeletal muscle tissue or the disclosed cells are used.

### BACKGROUND OF THE INVENTION

The human body is composed of 35-40% skeletal muscle, enabling breathing, posture and movement. A healthy skeletal muscle can fully regenerate from small injuries such as lacerations or cuts, since the muscle stem cells, also called satellite cells (SCs), can fully regenerate the injured tissue. However, major injuries do not heal, leaving permanent damage.

The current theory of “tissue engineering” consists in generating the required cell types and differentiating them in an engineered environment to produce an in vivo-like tissue. In tissue engineering, it is important to note that the extracellular environment is lost during a dissociation of differentiated cells, and thus developmentally relevant information may be lost. For example, dissociation breaks cell-cell interconnectivity, the geometric cell positioning, and the cell-extracellular matrix connectivity. This environment must be rebuilt during tissue engineering (Zimmermann et al. 2004, Tiburcy et al. 2017). Moreover, a differentiated skeletal muscle tissue consists not only of skeletal muscle fibers, but also of stromal/connective tissue cells, especially satellite cells, which form according to their environment and chemical stimuli.

The skilled person knows different tissue engineering methods related to skeletal muscle cells that use 2D cell cultures, small animal models, or extracted muscle tissue from small animals (Beldjilali-Labro et al. (2018) and Khodabukus et al. (2018)). For example, Chal et al. (2016) describe the production of muscle fibers in a 2D method.

In the past, small animal models have often been used to study biological processes. However, animal models generally have some limitations. With animal models, it is fundamentally questionable whether the results can be transferred to humans, especially with regard to disease/healing processes and drug efficacy.

To overcome the limitation of animal models, the production of engineered skeletal muscle cells and/or skeletal muscle tissues promises to be highly beneficial.

To support the differentiation of stem cells into skeletal muscle cells, stem cells have often been transfected with muscle-specific transcription factors in the past. For example, Rao et al. (2018) describe the production of an engineered skeletal muscle tissue wherein the Pax7 transgene is transiently overexpressed. However, the transfection rate differs for different cells and may vary with each experiment. In addition, many researchers use blood serum during the differentiation protocols. However, it is often unclear which factors are present in mammalian-derived blood serum and how they affect the differentiation. Thus, differentiation protocols using transgenes or serum have weaknesses, as the reproducibility of these methods is severely limited. Therefore, it is essential to develop a method in which human pluripotent stem cells are differentiated and matured into skeletal muscle cells and satellite cells or skeletal muscle tissue by defined factors, wherein no transgenes or serum are needed.

Furthermore, increasing evidence suggests that not only chemical stimuli but also physical stimuli play a role in skeletal muscle tissue development. Thus, a combination of chemical and physical stimuli in addition to surface topography and structural composition appears to be a possibility to reliably produce functional muscle tissue in vitro (Liao et al. (2008), Pavesi et al. (2015)). However, the chronology, duration, and identity of these different stimulations during differentiation and maturation of the cells remain unclear.

The development of a robust differentiation and maturation protocol is a very important step to enable a production of skeletal muscle cells and satellite cells as well as engineered skeletal muscle tissue.

The inventors of patent application WO 2017/100498A1 disclose a serum-free differentiation protocol of human pluripotent stem cells into skeletal myoblasts in a 2D method. However, this procedure requires an enrichment step of skeletal myoblasts via flow cytometry to remove non-differentiated cell types from the cell pool. A purification via flow cytometry does not allow for scaling, is associated with infection risks and very high cell loss, and therefore represents a key barrier to commercial application of cell products.

No method has been successfully described yet for an efficient differentiation of pluripotent stem cells—which is transgenic and serum-free—in which no further enrichment step is required for a specific cell type. Specifically, a method for an efficient differentiation of pluripotent stem cells into a skeletal muscle tissue that describes the chemical and physical stimuli while avoiding transgenes and serum has not yet been successfully described.

Shahriyari et al. (2018) merely report the production of a preliminary engineered skeletal muscle tissue. However, Shahriyari et al. lack information on essential features necessary for the production of engineered skeletal muscle tissue of the present invention. Kramer et al. (2014) describe the production of an engineered skeletal muscle from rat myoblasts, and not from pluripotent stem cells.

### SUMMARY OF THE INVENTION

The present invention describes methods for the preparation of engineered skeletal muscle tissue as well as skeletal myoblasts, skeletal myotubes and satellite cells, wherein the used media are serum-free, and the different chemical substances and their concentrations as well as the physical stimulation are defined. In addition, the method described herein does without a transfection of the human cells with transgenes. The engineered skeletal muscle cells exhibit myoblast-specific, myotube-specific, or satellite cell-specific gene markers, demonstrating the efficient differentiation of these cell types. The skeletal muscle tissue has, despite its engineered production, a very good stimulus-dependent contractility and shows contractions in response to different stimulation frequencies.

The invention includes methods in which pluripotent stem cells are differentiated and matured into skeletal myoblasts, skeletal myotubes, and satellite cells or skeletal muscle tissue. The skeletal muscle tissue is dispersed/embedded in an extracellular matrix.

The present invention relates to a method for producing engineered skeletal muscle tissue from pluripotent stem cells, comprising the steps of
(i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
(ii) inducing the myogenic specification by culturing the cells obtained in step (i) in a basal medium comprising an effective amount of (a) a gamma-secretase/NOTCH inhibitor, (b) FGF2, and (c) a serum-free additive as in (i), followed by
   continuing the cultivation in the medium with the addition of an effective amount of (d) HGF, followed by
   culturing the cells in a basal medium comprising an effective amount of (a) a gamma secretase/NOTCH inhibitor, (b) HGF, (c) a serum-free additive as in (i), and (d) knockout serum replacement (KSR);
(iii) expanding and maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR);
(iv) maturing the cells into skeletal myotubes and satellite cells by culturing the cells obtained in step (iii), which are dispersed in an extracellular matrix, under mechanical stimulation in a basal medium, comprising an effective amount of (a) a serum-free additive as in step (i) and (b) an additional serum-free additive comprising albumin, transferrin, ethanolamine, selenium or a bioavailable salt thereof, L-carnitine, fatty acid additive, and triiodo-L-thyronine (T3);
thereby producing engineered skeletal muscle tissue.

In addition, the present invention relates to a method for producing skeletal myoblasts, skeletal myotubes, and satellite cells from pluripotent stem cells, comprising the steps of
(i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
(ii) inducing myogenic specification by culturing the cells obtained in step (i) in a basal medium comprising an effective amount of (a) a gamma-secretase/NOTCH inhibitor, (b) FGF2, and (c) a serum-free additive as in (i), followed by
   continuing the cultivation in the medium with the addition of an effective amount of (d) HGF, followed by
   culturing the cells in a basal medium comprising an effective amount of (a) a gamma-secretase/NOTCH inhibitor, (b) HGF, (c) a serum-free additive as in (i), and (d) knockout serum replacement (KSR);
(iii) maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR), followed by
(iv) maturing the cells into skeletal myotubes and satellite cells by culturing the cells obtained in step (iii) in a basal medium comprising an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3)
   thereby producing skeletal myoblasts, skeletal myotubes, and satellite cells.

Furthermore, the present invention relates to engineered skeletal muscle tissue having multinuclear mature skeletal muscle fibers with satellite cells, and having no blood supply and/or no central nervous system control. In this regard, the presence of skeletal muscle fibers can be detected by staining of actinin and with DAPI.

In addition, the present invention is directed to mesodermally differentiated skeletal myoblast progenitor cells, prepared and obtained according to step (i) and characterized by the expression of the genes MSGN1 and/or TBX6, wherein the expression of MSGN1 and/or TBX6 can be determined by flow cytometry and/or immunostainings. These cells express the mRNA SP5, wherein the expression of SP5 can be determined by RNA sequencing.

In addition, the invention relates to a myogenically specified skeletal myoblast progenitor cell, produced and obtained according to steps (i) to (ii) and characterized by expression of the gene PAX3, wherein the expression of PAX3 can be determined by flow cytometry and/or immunostainings. These cells express the mRNA SIM1, wherein the expression of SIM1 can be determined by RNA sequencing.

Furthermore, the invention relates to skeletal myoblast cells, produced and obtained according to steps (i) to (iii) and characterized by the expression of actinin, wherein the expression of actinin can be determined by flow cytometry and/or immunostainings in skeletal myoblasts.

The invention further relates to satellite cells, prepared and obtained according to steps (i) to (iii), characterized by the expression of the gene Pax7, wherein the expression of Pax7 can be determined by flow cytometry and/or immunostainings, more preferably wherein satellite cells further express Ki67. Furthermore, a mixture of skeletal myoblast cells and satellite cells is according to the present invention, wherein a proportion of satellite cells out of the amount of all available cells of at least 10% is obtained, preferably at least 15%, more preferably at least 20%, even more preferably at least 30%, determined by the expression of Pax7 by flow cytometry; and/or wherein a proportion of skeletal myoblasts out of the amount of all available cells of at least 40% is obtained, preferably at least 50%, more preferably at least 60%, most preferably at least 70%, determined by expression of actinin by flow cytometry.

Furthermore, the present invention relates to skeletal myotubes, prepared and obtained according to steps (i) to (iv) and characterized by an anisotropic orientation of the actinin protein-containing sarcomere structure.

Further disclosed is the use of a skeletal muscle tissue according to the invention, and/or cells according to the invention, and/or skeletal myotubes according to the invention in an in vitro drug assay. The drug test may be a toxicity assay or an assay for skeletal muscle tissue function under the influence of pharmacological and gene therapeutic drug candidates.

In addition, the invention relates to skeletal muscle tissue and/or cells, and/or of skeletal myotubes according to the invention for use in medicine.

More specifically, the invention relates to satellite cells according to the invention for use in the therapy of damaged skeletal muscle and/or in the treatment of skeletal muscle diseases, preferably genetic skeletal muscle defects, in particular Duchenne muscular dystrophy and/or Becker-Kiener muscular dystrophy, and/or lysosomal storage diseases, in particular Pompe disease, preferably wherein the skeletal muscle disease is Duchenne muscular dystrophy.

Finally, the invention relates to the following in vitro methods:

An in vitro method for testing the efficacy of a drug candidate on a skeletal muscle tissue, comprising the steps of
(a) providing a skeletal muscle tissue according to the present invention,
(b) optionally inflicting damage on the skeletal muscle tissue, and
(c) contacting the skeletal muscle tissue of step (a) or (b) with a drug candidate;
preferably wherein the method further comprises determining the contraction force and/or structure of the skeletal muscle tissue and/or metabolic function and/or molecular parameters and/or protein biochemical parameters before and/or after step (c).

An in vitro method for testing the toxicity of a substance on a skeletal muscle tissue, comprising the steps of
(a) providing a skeletal muscle tissue according to the present invention,
(b) contacting the skeletal muscle tissue from step (a) with a substance to be tested.
preferably wherein the method further comprises determining the contraction force and/or structure of the skeletal muscle tissue and/or metabolic function and/or molecular parameters and/or protein biochemical parameters before and/or after step (b).

An in vitro method for testing the effect of nutrients and dietary supplements on skeletal muscle tissue performance, comprising the steps of
(a) providing a skeletal muscle tissue according to the present invention,
(b) contacting the skeletal muscle tissue from step (a) with a nutrient or dietary supplement to be tested,
preferably wherein the method further comprises determining the contraction force and/or structure of the skeletal muscle tissue and/or the metabolic function and/or molecular parameters and/or protein biochemical parameters before and/or after step (b).

An in vitro method for testing the efficacy of a drug candidate on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the present invention,
(b) optionally inflicting damage on the cells from step (a), and
(c) contacting the cells of step (a) or (b) with a drug candidate;
preferably wherein the method further comprises determining the expression of actinin and/or Pax7 before and/or after step (c), wherein the expression can be determined by flow cytometry and/or immunostainings.

An in vitro method for testing the toxicity of a substance to mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the present invention,
(b) contacting the cells of step (a) with a substance to be tested,
preferably wherein the method further comprises determining the expression of actinin and/or Pax7 before and/or after step (b), wherein the expression can be determined by flow cytometry and/or immunostainings.

An in vitro method for testing the effect of nutrients and dietary supplements on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the present invention,
(b) contacting the cells of step (a) with a nutrient or dietary supplement to be tested,
preferably wherein the method further comprises determining the expression of actinin and/or Pax7 before and/or after step (b), wherein the expression can be determined by flow cytometry and/or immunostainings.

### DETAILED DESCRIPTION OF THE INVENTION

The present disclosure relates to a method for producing engineered skeletal muscle tissue from pluripotent stem cells, comprising the steps of
(i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
(ii) inducing the myogenic specification by culturing the cells obtained in step (i) in a basal medium comprising an effective amount of (a) a gamma-secretase/NOTCH inhibitor, (b) FGF2, and (c) a serum-free additive as in (i), followed by
   continuing the cultivation in the medium with the addition of an effective amount of (d) HGF, followed by
   culturing the cells in a basal medium comprising an effective amount of (a) a gamma secretase/NOTCH inhibitor, (b) HGF, (c) a serum-free additive as in (i), and (d) knockout serum replacement (KSR);
(iii) expanding and maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR);
(iv) maturing the cells into skeletal myotubes and satellite cells by culturing the cells obtained in step (iii), which are dispersed in an extracellular matrix, under mechanical stimulation in a basal medium, comprising an effective amount of (a) a serum-free additive as in step (i) and (b) an additional serum-free additive comprising albumin, transferrin, ethanolamine, selenium or a bioavailable salt thereof, L-carnitine, fatty acid additive, and triiodo-L-thyronine (T3);
thereby producing engineered skeletal muscle tissue.

In a preferred embodiment, the pluripotent stem cells are of primate origin, in particular human pluripotent stem cells. In a particularly preferred embodiment, the pluripotent stem cells are selected from induced pluripotent stem cells, embryonic stem cells, parthenogenetic stem cells, pluripotent stem cells produced via nuclear transfer, and pluripotent cells produced via chemical reprogramming, in particular wherein the pluripotent stem cells are induced pluripotent stem cells.

“Pluripotent stem cells” are capable of differentiating into any cell type of the body. Therefore, human pluripotent stem cells offer a considerable possibility of obtaining, for example, skeletal myoblasts, skeletal myotubes and satellite cells. Currently, the most commonly used pluripotent cells are induced pluripotent stem cells (iPSCs) or embryonic stem cells (ESCs). Human ESC lines were first produced by Thomson et al (Thomson et al, Science 282: 1145-1147 (1998)). Nowadays, human ESC research enables the development of a new technology to reprogram somatic cells into an ES-like cell. This technology was developed by Yamanaka et al. in 2006 and is also applicable to human cells (Takahashi & Yamanaka Cell 126: 663-676 (2006) and Takahashi, Kazutoshi et al. Cell, 131:(5)861-872(2007)). The resulting induced pluripotent cells (iPSC) show very similar behavior to ESC and are also capable of differentiating into any cell of the body. Furthermore, in another embodiment, parthenogenetic stem cells can be used. Parthogenetic stem cells can be derived in mammals, preferably in mice as well as humans, from blastocysts that develop after in vitro activation of unfertilized oocytes. These cells exhibit the key features of pluripotent stem cells, such that they are able to differentiate into any cell type in vitro (Espejel S et al. (2014)). Accordingly, the pluripotent stem cells can be selected from induced pluripotent stem cells, embryonic stem cells, and parthenogenetic stem cells. However, in the context of the present invention, the pluripotent stem cells are not produced by a method in which the genetic identity of a human is altered in the germline, or in which a human embryo is used for industrial or commercial purposes. In a particularly preferred embodiment, induced pluripotent stem cells are selected.

In order to achieve a directed cell differentiation of the pluripotent stem cells into skeletal muscle tissue, differentiation is achieved with the aid of specific factors or additives. In general, the differentiation steps according to the invention are carried out in the presence of a “basal medium”. Any suitable basal medium can be used for the method. Preferably, the basal medium used in steps (i)-(iv) is selected from DMEM, DMEM/F12, RPMI, IMDM, alphaMEM, medium 199, Hams F-10 and Hams F-12. Preferably, the basal medium used in steps (i)-(iv) is DMEM supplemented with pyruvate. Even more preferably, the basal medium used in steps (i)-(iv) is DMEM supplemented with pyruvate containing 1 g/l glucose. Basal media are commercially available or can be prepared according to publicly available formulations, e.g. from catalogs of the ATCC. In a strongly preferred embodiment, the basal medium is DMEM with 1 g/l glucose plus a glutamine preparation (e.g., L-alanyl-L-glutamine or GlutaMAX™) and consists of the substances listed in Table 3. If deemed appropriate, the basal medium may be supplemented with an effective concentration of non-essential amino acids. In a preferred embodiment, the basal medium is supplemented with a single effective concentration of the non-essential amino acids listed in Table 2. The basal medium in steps (ii), (iii) and (iv) may be independently selected from the basal medium used in step (i). However, in a preferred embodiment, the basal medium in steps (i)-(iv) is the same.

In general, the different differentiation stages of steps (i)-(iv) can be detected using expressed genes that are characteristic of a particular stage. One method that can measure gene expression is RNA sequencing (RNA-Seq). RNA sequencing is also called transcriptome analysis. RNA sequencing is the determination of the nucleotide sequence of RNA, based on high-throughput methods. For this purpose, RNA is converted (transcribed) into cDNA so that the DNA sequencing method can be applied. Thus, RNA sequencing provides information about which mRNAs are expressed and is characterized by low background noise, higher resolution, and high replication rates. The person skilled in the art is familiar with and can perform the method of mRNA sequencing. Example 1 of the present invention shows exemplary data measured using RNA sequencing. Specifically, FIG. 4 shows a time course of mRNA expression of different genes in a time frame of 0 to 60 days during the differentiation protocol of the invention.

The mRNA expression of NANOG, POU5F1 (OCT4), and ZFP42 is characteristic of pluripotent stem cells. This means that cells expressing these markers are pluripotent.

During the differentiation of pluripotent stem cells according to the invention, “mesodermal differentiation” is induced by specific factors/additives in step (i). In all bilaterian animals (bilateria), as well as humans, the mesoderm is one of the three primary germ layers in the very early embryo. In bilaterian animals, there are three major components of the mesoderm: the paraxial mesoderm, the intermediate mesoderm, and the lateral plate mesoderm. The paraxial mesoderm in bilaterian animals gives rise to skeletal muscle, among other things. Induction of mesodermal differentiation is characterized by gene expression of specific genes, such as the mRNAs of MSGN1, TBX6, and MEOX1. An mRNA expression of these or other genes specific for paraxial mesoderm expression can be measured by RNA sequencing as described herein.

As set forth above, the basal medium of step (i) comprises an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof. It is known to the skilled person that an effective concentration or amount of a receptor/enzyme agonist or inhibitor varies with the availability and biological activity of the respective substance.

In one embodiment, the effective amount of FGF2 is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml.

Glycogen synthase kinase 3 (GSK-3) is a serine/threonine protein kinase that selectively adds phosphate residues to the serine and threonine residues of other proteins. The inhibition of glycogen synthase kinase 3 (GSK3) helps activate the Wnt signalling pathway for the differentiation of pluripotent stem cell. For example, the GSK3 inhibitor in the basal medium is selected from the group consisting of CHIR99021, CHIR98014, SB216763, TWS119, tideglusib, SB415286, 6-bromoindirubin-3-oxime, and a valproate salt, with the GSK3 inhibitor CHIR99021 being preferred. However, any GSK3 inhibitor suitable for the method of the invention may be used. When the GSK3 inhibitor is CHIR99021, an effective amount is 1-20 μM, preferably 2-19 μM, more preferably 3-18 μM, even more preferably 4-17 μM, even more preferably 5-16 μM, even more preferably 6-15 μM, even more preferably 7-14 μM, even more preferably 7.5-13 μM, even more preferably 8-12 μM, even more preferably 9-11 μM, and most preferably about 10 μM.

A SMAD inhibitor inhibits proteins that are of critical importance for regulating cell development and growth. For example, the SMAD inhibitor in the basal medium is selected from the group consisting of LDN193189, K02288, LDN214117, ML347, LDN212854, DMH1, wherein preferably the SMAD inhibitor is LDN193189. However, any SMAD inhibitor suitable for the method of the invention may be used. When the SMAD inhibitor is LDN193189, an effective amount is 0.05-5 μM, preferably 0.1-2.5 μM, more preferably 0.2-1 μM, even more preferably 0.25-0.8 μM, even more preferably 0.3-0.75 μM, even more preferably 0.35-0.7 μM, even more preferably 0.4-0.6 μM, even more preferably 0.45-0.55 μM, and most preferably about 0.5 μM.

It is known to a skilled person that an effective concentration or amount of an inhibitor varies with the availability and biological activity of the respective substance and this applies to all substances, such as proteins/peptides, nucleotides or chemical compounds.

In one embodiment, the serum-free additive is provided in steps (i), (ii), (iii) and (iv) of the method at a final concentration in the medium of 50-500 μg/ml transferrin (preferably 70-300 μg/ml transferrin, more preferably 80-200 μg/ml transferrin, even more preferably 90-150 μg/ml transferrin, most preferably about 100 μg/ml transferrin),

1-25 μg/ml insulin (more preferred 2-13 μg/ml insulin, more preferred 3-10 μg/ml insulin, more preferred 4-6 μg/ml insulin, most preferred about 5 μg/ml insulin), 0.001-0.1 μg/ml progesterone (preferably 0.002-0.05 μg/ml progesterone, more preferably 0.004-0.01 μg/ml progesterone, even more preferably 0.005-0.008 μg/ml progesterone, most preferably about 0.0063 μg/ml progesterone), 5-50 μg/ml putrescine (preferably 10-35 μg/ml putrescine, more preferably 12-25 μg/ml putrescine, even more preferably 14-18 μg/ml putrescine, most preferably about 16 μg/ml putrescine); and

6-600 nM selenium (preferably 12-300 nM selenium, more preferably 20-150 nM selenium, even more preferably 25-50 nM selenium, most preferably about 30 nM selenium) or a bioavailable salt thereof. In a preferred embodiment, selenium is present as selenite, wherein an effective concentration thereof is 1-30 μg/l selenite (preferably 2-20 μg/l selenite, more preferably 3-10 μg/l selenite, even more preferably 4-6 μg/l selenite, most preferably about 5 μg/l selenite) in the medium.

A serum-free additive that meets the above requirements can be purchased commercially. For example, N2 additive can be used. In a preferred embodiment, the serum-free additive is N2 additive at a concentration 0.1-10% (v/v) N2 additive, preferably 0.3-7.5% (v/v) N2 additive, more preferably 0.5-5% (v/v) N2 additive, more preferably 0.75%-2% (v/v) N2 additive, more preferably 0.9%-1.2% (v/v) N2 additive, and most preferably about 1% (v/v) N2 additive. The N2 additive is commercially available in a 100-times effective concentration, and the composition is listed in Table 1. This means that 1% (v/v) of the N2 additive corresponds to a single effective concentration.

In a preferred embodiment, step (i) of the method is carried out for 24 to 132 hours, preferably for 48 to 120 hours, more preferably for 60 to 114 hours, even more preferably for 72 to 108 hours, more preferably for 84 to 102 hours, and most preferably for about 96 hours. The duration of step (i) and the concentration of the substances (a) FGF2, (b) a GSK3 inhibitor, (c) a SMAD inhibitor, and (d) a serum-free additive may be optimized by monitoring the efficiency of the induction of mesoderm differentiation. As described above, the efficiency of mesoderm differentiation can be tracked by RNA sequencing. The induction of mesoderm differentiation is given if, for example, one or more of the gene markers MSGN1, TBX6 and MEOX1 have an expression value at least 5-fold higher compared to the pluripotent stem cell (preferably an expression value at least 10-fold higher, more preferably an expression value 20-fold higher, even more preferably an expression value at least 30-fold higher, most preferably an expression value at least 50-fold higher), as measured by “reads per kilobase million” by RNA sequencing.

In step (ii) of the method according to the invention, the “myogenic specification” is induced. This differentiation stage is characterized by the expression of specific factors. For example, the mRNA Pax3 is expressed in myogenic specification, the expression of which can be determined by RNA sequencing (see FIGS. 1 and 2 for a schematic overview; see FIG. 4 for experimental data on the expression of PAX3). A myogenic specification is given if, for example, the gene marker Pax3 has an expression value at least 5-fold higher compared to the pluripotent stem cell (preferably an expression value at least 10-fold higher, more preferably an expression value 20-fold higher, even more preferably an expression value 30-fold higher), as measured by “reads per kilobase million” by RNA sequencing.

As described above, step (ii) includes three cultivation steps. Particularly, step (ii) comprises culturing the cells obtained from step (i) in basal medium in an effective amount of (a) a gamma-secretase/NOTCH inhibitor, (b) FGF2, and (c) a serum-free additive as in (i), followed by continuing the cultivation in the medium with the addition of an effective amount of (d) HGF, followed by culturing the cells in a basal medium comprising an effective amount of (a) a gamma-secretase/NOTCH inhibitor, (b) HGF, (c) a serum-free additive as in (i), and (d) knockout serum replacement (KSR).

As in step (i), the basal medium in step (ii) may be selected from DMEM, DMEM/F12, RPMI, IMDM, alphaMEM, Medium 199, Hams F-10, and Hams F-12. In addition, the basal medium may be supplemented with non-essential amino acids and/or pyruvate. Exemplary and preferred embodiments for the basal medium in step (ii) may be selected analogous to the exemplary and preferred embodiments in step (i). The basal medium in step (ii) may be independently selected from the basal medium used in step (i). However, in a preferred embodiment, the basal medium in steps (i) and (ii) is the same.

For example, the gamma-secretase/NOTCH inhibitor is selected from the group consisting of DAPT, R04929097, semagacestat (LY450139), avagacestat (BMS-708163), dibenzazepine (YO-01027), LY411575, IMR-1, L685458, preferably wherein the gamma-secretase/NOTCH inhibitor is DAPT. However, any gamma-secretase/NOTCH inhibitor suitable for the method of the invention may be used.

When the gamma-secretase/NOTCH inhibitor is DAPT, its effective amount is 1-20 μM, preferably 2-19 μM, more preferably 3-18 μM, even more preferably 4-17 μM, even more preferably 5-16 μM, even more preferably 6-15 μM, even more preferably 7-14 μM, even more preferably 7.5-13 μM, even more preferably 8-12 μM, even more preferably 9-11 μM, and most preferably about 10 μM.

In step (ii), the effective amount of FGF2 is, for example, 15-30 ng/ml, preferably 17.5-25 ng/ml, more preferably 18-22 ng/ml, even more preferably 19-21 ng/ml, and most preferably about 20 ng/ml.

For example, an effective amount of HGF is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml.

It is known to a skilled person that an effective concentration or amount of a receptor/enzyme agonist or inhibitor varies with the availability and biological activity of the respective substance.

As used herein, the term “knockout serum replacement” (KSR) means an effective concentration of ascorbic acid, insulin, transferrin, and albumin. In a preferred embodiment, the KSR additionally comprises an effective concentration of selenium or a bioavailable salt thereof, glutathione, and trace elements. In a more preferred embodiment, the KSR comprises an effective concentration of the substances listed in Table 5. In the most preferred embodiment, the KSR comprises the substances of Table 5 at the concentration indicated. The “knockout serum replacement” (KSR) is known in the prior art and can be prepared according to the formulation on pages 27-29 of patent application WO 98/30679. Alternatively, KSR is commercially available, e.g. from Gibco.

In a preferred embodiment, the KSR is used in an amount of 5-20% (v/v), preferably 6-17.5% (v/v), more preferably 7-15% (v/v), more preferably 8%-12% (v/v), more preferably 9%-11% (v/v), and most preferably about 10% (v/v) KSR. In a strongly preferred embodiment, the KSR is used in the presence of a reducing agent. Any suitable reducing agent may be used, and examples of reducing agents are beta-mercaptoethanol and/or alpha-thioglycerol. Beta-mercaptoethanol is typically used at a concentration of 0.02-0.5 mM, more preferably at a concentration of 0.05-0.02 mM, most preferably at a concentration of about 0.1 mM. Alternatively, alpha-thioglycerol may be used, for example at a concentration of 0.02-0.5 mM, more preferably at a concentration of 0.05-0.02 mM, most preferably at a concentration of about 0.1 mM.

In one embodiment, the cultivation in step (ii) is carried out in the presence of (a) a gamma-secretase/NOTCH inhibitor, (b) FGF2, and (c) the serum-free additive for 36 to 60 hours, preferably for 42 to 54 hours, and most preferably for about 48 hours; and/or

the cultivation is carried out in the presence of (a) a gamma-secretase/NOTCH inhibitor, (b) FGF2, (c) the serum-free additive, and (d) HGF for 36 to 60 hours, preferably for 42 to 54 hours, and most preferably for about 48 hours; and/or the cultivation is carried out in the presence of (a) a gamma-secretase/NOTCH inhibitor, (b) HGF, (c) the serum-free additive, and (d) knockout serum replacement (KSR) for 72 to 120 hours, preferably for 76 to 114 hours, more preferably for 84 to 108 hours, even more preferably for 90 to 102 hours, and most preferably for about 96 hours.

In step (iii) of the method of the invention, the cells are advantageously matured and expanded into skeletal myoblasts and satellite cells. Skeletal myoblasts are characterized by being fusion competent and therefore able to fuse into skeletal myotubes in a further step. Satellite cells, also called muscle stem cells, are small multipotent cells. Satellite cells are capable of giving rise to (i) satellite cells or (ii) differentiated skeletal myoblasts. More specifically, after activation, satellite cells can re-enter the cell cycle to proliferate and differentiate into myoblasts. This differentiation stage of the method is characterized by the expression of specific factors. For example, the expression of Pax7 is characteristic of the presence of satellite cells. At the same time, the expression of MyoD is characteristic of skeletal myoblasts, the respective expression of which can be determined by RNA sequencing (see FIGS. 1 and 2 for a schematic overview; see FIG. 4 for experimental data on the expression of MyoD1 and PAX7). Skeletal myoblasts are present if, for example, the gene marker MyoD has at least a 5-fold higher expression value compared to the pluripotent stem cell (preferably at least a 10-fold higher expression value, more preferably a 15-fold higher expression value, even more preferably a 20-fold higher expression value), as measured by “reads per kilobase million” using RNA sequencing. Satellite cells are present if, for example, the gene marker PAX7 has an expression value at least 5-fold higher compared to the pluripotent stem cell (preferably an expression value at least 10-fold higher, more preferably an expression value 15-fold higher, even more preferably an expression value 20-fold higher), as measured by “reads per kilobase million” by RNA sequencing.

As described above, the basal medium of step (iii) comprises an effective amount of (a) HGF, (b) a serum-free additive as in (i), and (c) knockout serum replacement (KSR). As in steps (i) and (ii), the basal medium in step (iii) may be selected from DMEM, DMEM/F12, RPMI, IMDM, alphaMEM, Medium 199, Hams F-10, and Hams F-12, and the basal medium may be supplemented with non-essential amino acids and/or pyruvate. Exemplary and preferred embodiments for the basal medium in step (iii) may be selected analogously to the exemplary and preferred embodiments in step (i). The basal medium in step (iii) may be selected independently of the basal medium used in steps (i) and (ii). However, in a preferred embodiment, the basal medium in steps (i), (ii) and (iii) is the same.

The KSR and the optional reducing agent in step (iii) comprise the same preferred embodiments as the KSR and the optional reducing agent in step (ii). Thus, the KSR can either be prepared by the person skilled in the art or purchased commercially.

In step (iii), the effective amount of HGF is, for example, 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or

the KSR is used in an amount of 5-20% (v/v), preferably 6-17.5% (v/v), more preferably 7-15% (v/v), more preferably 8%-12% (v/v), more preferably 9%-11% (v/v), and most preferably about 10% (v/v) KSR; in particular wherein the KSR is used in the presence of a reducing agent such as beta-mercaptoethanol and/or alpha-thioglycerol.

It is known to the skilled person that an effective amount or effective concentration of a receptor/enzyme agonist or inhibitor varies with the availability and biological activity of the respective substance.

In step (iv) of the method according to the invention, the cells are advantageously matured into skeletal myotubes and satellite cells. Skeletal myotubes are formed by the fusion of skeletal myoblasts. Therefore, skeletal myotubes are multinuclear cellular structures formed by fusion of mature myoblasts into long thin myotubes. Skeletal myotubes are also called myocytes or muscle fibers. FIG. 2 provides a schematic overview of the developmental stages undergone by the engineered skeletal muscle tissue, and the formation of engineered skeletal muscle tissue is known in the prior art as myogenesis. Skeletal myotubes (muscle fibers) are characterized by an anisotropic orientation of the actinin-containing sarcomere structure. A regeneration-competent satellite cell niche forms adjacent to the fused skeletal myotubes. The satellite cell niche is external to the skeletal myotubes, but in close contact with the skeletal myotubes. The satellite cell niche is anatomically characteristic and also forms in natural skeletal muscle tissues. Consequently, together with the generated satellite cells, it represents another desirable quality feature for the engineered skeletal muscle tissue. This differentiation stage is characterized by the expression of specific factors, too. For example, the expression of Pax7 is characteristic of the presence of satellite cells. At the same time, the expression of myogenin and actinin is characteristic of skeletal myotubes, the respective expression of which can be determined by RNA sequencing (see FIGS. 1 and 2 for a schematic overview; see FIG. 4 for experimental data on the expression of PAX7 (paired box 7), ACTN2 (actinin alpha 2), DMD (dystrophin), and MYH3 (myosin heavy chain 3). Satellite cells are present if, for example, the mRNA of PAX7 has at least a 5-fold higher expression value compared to the pluripotent stem cell (preferably at least a 10-fold higher expression value, more preferably a 15-fold higher expression value, even more preferably a 20-fold higher expression value), as measured by “reads per kilobase million” by RNA sequencing. Skeletal myotubes are present if, for example, the gene marker ACTN2 has an expression value at least 5-fold higher compared to the pluripotent stem cell (preferably an expression value at least 50-fold higher, more preferably an expression value 100-fold higher, even more preferably an expression value 150-fold higher), measured by “reads per kilobase million” by RNA sequencing. In fact, the DMD and MYH3 gene markers typically have at least a 200-fold higher expression value compared to the pluripotent stem cell (preferably at least a 500-fold higher expression value, more preferably a 1000-fold higher expression value).

As described above, the cells obtained in step (iii) are dispersed in an extracellular matrix and are matured under mechanical simulation. Mechanical simulation can be performed, for example, with the aid of a stretching device, as is generally known and used in the art. Preferably, the stretching device exerts a static, phasic or dynamic strain. Thus, the mechanical strain may be (a) static, (b) phasic, or (c) dynamic. An example of a static strain is an isometric muscle contraction, in which the muscle undergoes only a change in tension and no change in length. Thus, no shortening occurs in the muscle during an isometric muscle contraction. A phasic strain can be a quasi-isotonic muscle contraction in which the muscle shortens during the contraction and the tension applied to the muscle remains the same. Dynamic strain can occur, for example, when the muscle is suspended on flexible supports to promote auxotonic contraction. In an auxotonic contraction, both the muscle length and the muscle tension change. Preferably, the mechanical stimulation in step (iv) is a static mechanical stimulation, i.e., static tension (static strain). This means that the cells from step (iii) and the extracellular matrix are under a force and an opposing force (counterforce).

As set forth above, the basal medium of step (iv) comprises an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3).

Exemplary and preferred embodiments for the basal medium in step (iv) may be selected analogously to the exemplary and preferred embodiments in step (i).

The additional serum-free additive in step (iv) of the method is formulated such that the additional serum-free additive provides a final concentration of the following substances: 0.5-50 mg/ml albumin (preferably 1-40 mg/ml, more preferably 2-30 mg/ml, even more preferably 3-20 mg/ml, even more preferably 4-10 mg/ml, and most preferably 4.5-7.5 mg/ml, such as about 5 mg/ml);

1-100 μg/ml transferrin (preferably 2-90 μg/ml, more preferably 3-80 μg/ml, even more preferably 4-70 μg/ml, even more preferably 5-60 μg/ml, more preferably 6-50 μg/ml, more preferably 7-40 μg/ml, more preferably 8-30 μg/ml, more preferably 9-20 μg/ml, such as about 10 μg/ml);

0.1-10 μg/ml ethanolamine (preferably 0.2-9 μg/ml, more preferably 0.3-8 μg/ml, even more preferably 0.4-7 μg/ml, even more preferably 0.5-6 μg/ml, more preferably 0.6-5 μg/ml, more preferably 0.7-4 μg/ml, more preferably 0.8-3 μg/ml, most preferably 1-2.5 μg/ml, such as about 2 μg/ml); 17.4-1744 nM selenium or a bioavailable salt thereof (preferably 35-850 nM, more preferably 70-420 nM, even more preferably 120-220 μg/ml, most preferably about 174 nM);

0.4-40 μg/ml L-carnitine HCl (preferably 0.5-30 μg/ml, more preferably 1-20 μg/ml, even more preferably 2-10 μg/ml, more preferably 3-5 μg/ml, and most preferably about 4 μg/ml);

0.05-5 μl/ml fatty acid additive (preferably 0.1-4 μl/ml, more preferably 0.2-3 μl/ml, even more preferably 0.3-3 μl/ml, more preferably 0.4-2 μl/ml, and most preferably 0.45-1 μl/ml, such as about 0.5 μl/ml); and

0.0001-0.1 μg/ml triiodo-L-thyronine (T3) (preferably 0.001-0.01 μg/ml, more preferably 0.002-0.0075 μg/ml, even more preferably 0.003-0.005 μg/ml, most preferably about 0.004 μg/ml).

The fatty acid additive may comprise, for example, linoleic acid and/or linolenic acid. In a preferred embodiment, the additional serum-free additive further comprises 0.1-10 μg/ml hydrocortisone (preferably 0.2-9 μg/ml, more preferably 0.3-8 μg/ml, even more preferably 0.4-7 μg/ml, even more preferably 0.5-6 μg/ml, even more preferably 0.6-5 μg/ml, even more preferably 0.7-4 μg/ml, even more preferably 0.8-3 μg/ml, most preferably 0.9-2 μg/ml, such as about 1 μg/ml). In a likewise preferred embodiment, the additional serum-free additive further comprises 0.3-30 μg/ml insulin (preferably 0.5-25 μg/ml, more preferably 1-20 μg/ml, even more preferably 1.5-15 μg/ml, even more preferably 2-10 μg/ml, most preferably 2.5-5 μg/ml, such as about 3 μg/ml). For example, a bioavailable salt of selenium is sodium selenite, such that a final concentration of 0.003-0.3 μg/ml (preferably 0.005-0.2 μg/ml, more preferably 0.01-0.1 μg/ml, even more preferably 0.02-0.05 μg/ml, and most preferably 0.03 μg/ml, such as about 0.032 μg/ml) is provided in the basal medium.

Furthermore, the additional serum-free additive may further comprise one or more components selected from the group consisting of hydrocortisone, ascorbic acid, vitamin A, D-galactose, linolenic acid, progesterone, and putrescine. These components are beneficial for cell viability. Suitable concentrations of the respective components are known to those skilled in the art or can be readily determined by routine measures.

An example of an additional serum-free additive mentioned in step (iv) can be prepared according to published protocols (see also Brewer et al. 1993) or purchased commercially. For example, B27 (Table 4) may be used. In a preferred embodiment, the B27 additive is used in an amount of 0.1-10% B27, preferably 0.5-8%, more preferably 1-6%, more preferably 1.5-4%, even more preferably 1.5-4%, and most preferably about 2% B27.

The invention may be preceded by a seeding step prior to step (i), and the engineered skeletal muscle tissue obtained is called bioengineered skeletal muscle (BSM). In the seeding step, the pluripotent stem cells are seeded in a stem cell medium in the presence of a ROCK inhibitor, preferably wherein the seeding step is performed 18-30 hours prior to step (i). For example, the ROCK inhibitor is selected from the group consisting of Y27632, H-1152P, thiazovivin, fasudil, hydroxyfasudil, GSK429286A and RKI1447, preferably the ROCK inhibitor is selected from the group consisting of Y27632, H-1152P, thiazovivin, fasudil, and hydroxyfasudil, more preferably the ROCK inhibitor is selected from the group consisting of Y27632 and H-1152P, wherein particularly preferably the ROCK inhibitor is Y27632. However, any ROCK inhibitor suitable for the method of the invention may be used. It is understood by those skilled in the art that the concentration of an effective amount of a ROCK inhibitor varies with the availability and inhibition constant of the inhibitor in question. For example, in the case of Y27632, the medium used in the seeding step may be used at a concentration of 0.5-10 μM, preferably 1-9 μM, more preferably 2-8 μM, more preferably 3-7 μM, more preferably 4-6 μM, and most preferably about 5 μM. A stem cell medium may be used in the seeding step, and in principle any stem cell medium suitable for the method may be used. Suitable stem cell media are known to the person skilled in the art, wherein the iPS-Brew XF stem cell medium is particularly preferred.

Furthermore, in the seeding step, the pluripotent stem cells may first be seeded into an engineered form in the presence of one or more components of an extracellular matrix in a master mix before the stem cell medium is added. In the seeding step, the pluripotent stem cells are dispersed into an extracellular matrix prior to step (i), so that the cells are embedded in the extracellular matrix to differentiate and mature into an engineered skeletal muscle tissue in a three-dimensional structure.

The “extracellular matrix” acts as a scaffold and provides a structural and functional microenvironment for cell growth and differentiation. Although the composition of the extracellular matrix is unique to each natural tissue, the major components of the extracellular matrix are collagens, fibronectin, laminin, and various types of glycoaminoglycans and proteoglycans. Proteoglycans form a class of particularly strongly glycosylated glycoproteins that accomplish a stabilization between the cells of an organism. Here, they form large complexes, both with other proteoglycans and with hyaluronic acid, as well as with proteins such as collagen—the main component of the extracellular matrix. Laminin is a glycoprotein similar to collagen. Fibronectin is also a glycoprotein that is important for extracellular collagen polymerization, and may play an important role in tissue repair, among other things. The component of an extracellular matrix in the master mix is preferably collagen, preferably type I collagen, more preferably of bovine origin, human origin, or marine origin, particularly collagen of bovine origin. Optionally, the extracellular matrix additionally comprises laminin and/or fibronectin.

The pluripotent stem cells are typically seeded in medium at a ratio of 1-6×10⁶ cells/ml and 0.7-1.4 mg/ml collagen. In one embodiment, the master mix comprises 5-15% (v/v), preferably 7.5%-12.5% (v/v), more preferably 9-11% (v/v), and most preferably about 10% (v/v) of an exudate of Engelbreth-Holm-Swarm (EHS) mouse sarcoma cells as an extracellular matrix component. In a particularly preferred embodiment, the exudate is Matrigel. The pH of the master mix is typically between pH 7.2 and pH 7.8. Matrigel is known to the skilled person and is further described in the prior art (Hughes et al. 2010).

As an alternative to an exudate of EHS mouse sarcoma cells, the master mix may comprise stromal cells, wherein the stromal cells generate the extracellular matrix components collagens, laminin, fibronectin, and/or proteoglycans. The pH of the master mix is typically between pH 7.2 and pH 7.8.

In a preferred embodiment, the stem cell medium is added to the master mix in the engineered form after about one hour, and the stem cell medium preferably comprises an effective concentration of KSR and FGF2. For example, the stem cell medium may comprise 5-20% (v/v), preferably 6-17.5% (v/v), more preferably 7-15% (v/v), more preferably 8%-12% (v/v), more preferably 9%-11% (v/v), and most preferably about 10% (v/v) KSR.

An effective amount of FGF2 is typically 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml FGF2.

When making BSM, step (iii) is carried out for 7-11 days, preferably for 8-10 days, and most preferably for about 9 days.

Alternatively, the skeletal myoblasts and satellite cells can be seeded in an additional step after step (iii) and before step (iv), and the obtained engineered skeletal muscle tissue is called engineered skeletal muscle (ESM). Herein, the skeletal myoblasts and satellite cells are seeded into an engineered form in the presence of one or more components of an extracellular matrix in a master mix. Preferably, the component of an extracellular matrix in the master mix is collagen, preferably type I collagen, more preferably of bovine origin, human origin or marine origin, in particular collagen of bovine origin, optionally wherein the extracellular matrix additionally comprises laminin and/or fibronectin. In a seeding step after step (iii) and prior to step (iv), the skeletal myoblasts and satellite cells are seeded, for example, at a ratio of 1-10×10⁶ cells/ml and 0.7-1.4 mg/ml collagen in medium.

In one embodiment, the master mix comprises 5-15% (v/v), preferably 7.5%-12.5% (v/v), more preferably 9-11% (v/v), and most preferably about 10% (v/v) of an exudate of Engelbreth-Holm-Swarm (EHS) mouse sarcoma cells as an extracellular matrix component. In a particularly preferred embodiment, the exudate is Matrigel. The pH of the master mix is typically between pH 7.2 and pH 7.8.

As an alternative to an exudate of EHS mouse sarcoma cells, the master mix may comprise stromal cells, wherein the stromal cells generate the extracellular matrix components collagens, laminin, fibronectin, and/or proteoglycans. The pH of the master mix is typically between pH 7.2 and pH 7.8.

In a preferred embodiment, after about one hour, the basal medium used as in step (iii) is added to the master mix in the engineered form, wherein the medium additionally comprises an effective amount of a ROCK inhibitor. For example, the ROCK inhibitor is selected from the group consisting of Y27632, H-1152P, thiazovivin, fasudil, hydroxyfasudil, GSK429286A, and RKI1447. Preferably, the ROCK inhibitor is selected from the group consisting of Y27632, H-1152P, thiazovivin, fasudil, and hydroxyfasudil. More preferably, the ROCK inhibitor is selected from the group consisting of Y27632 and H-1152P, wherein particularly preferred the ROCK inhibitor is Y27632. However, any ROCK inhibitor suitable for the method of the invention may be used. It is known to a person skilled in the art that the concentration of an effective amount of a ROCK inhibitor varies with the availability and inhibition constant of the inhibitor in question. For example, in the case of Y27632, the medium used in the seeding step may be used in a concentration of 0.5-10 μM, preferably 1-9 μM, more preferably 2-8 μM, more preferably 3-7 μM, more preferably 4-6 μM, and most preferably about 5 μM.

When preparing ESM, about one day after the seeding step, which occurs between step (iii) and step (iv), the medium is exchanged for a medium as used in step (iii), and the cells are then further cultured in this medium for another 5-9 days, preferably 6-8 days, most preferably about 7 days.

In the preparation of a BSM or an ESM, the engineered form may be, for example, in the form of a ring, a ribbon, a strand, a patch, a pouch, or a cylinder, optionally wherein individual skeletal muscle tissues may be fused. That means that individual and/or different geometries can be fused to form a skeletal muscle tissue, and thus different muscle shapes can be achieved. Especially the form of a ring, a strand or a ribbon is useful for applications in in vitro methods, e.g. for toxicity testing. Typically, the engineered form is obtained by casting the master mix, so that generally, any desired castable engineered form can be produced.

Step (iv) can be carried out for at least 19 days, preferably at least 28 days, more preferably for at least 56 days, even more preferably for at least 120 days, and especially for at least 240 days, wherein a longer cultivation is possible. The inventors have already been able to carry out a cultivation of 240 days (8 months), although there is nothing speaking against a longer cultivation period.

In contrast to many methods disclosed in the prior art, the method according to the invention does not comprise a transfection step with a differentiation- or maturation-related transgene. Preferably, the method does not comprise a myogenic transgene and more preferably, the method does not comprise the Pax7 or MyoD transgene. A “transgene” refers to a gene introduced into a cell. Such a transgene may be transfected into the cell in the form of DNA (e.g., in the form of a plasmid) or RNA. The transgene is then expressed in the cell, thereby altering the cell's characteristics. For example, transcription factors can be introduced into the cell as transgenes, which then affect the expression of other genes. Thus, a myogenic transgene can increase the proportion of skeletal myoblasts in a cell population. However, transfection experiments with a transgene, such as Pax7 or MyoD, have different transfection efficiencies depending on the experiment and cell type. This makes methods that require a transfection step less controllable and thus less reproducible. Thus, a transgene-free method is advantageous over a method that requires transfection with a transgene. However, it cannot be ruled out that the pluripotent stem cells are genetically modified in another form, for example, to simulate a disease pattern. Furthermore, the genetically engineered labelling of cell types and/or cell functions (e.g. calcium or voltage signals) or the control of the cell function via e.g. optogenetic mechanisms (e.g. contraction frequency) is not excluded.

Another advantage of the method according to the invention is that no further selection step of specific cell types is required, such as e.g. skeletal myoblasts. Preferably, the method does not include an enrichment step by cell selection, more preferably no enrichment step by antibody-based cell selection. This is advantageous because the cells do not need to be extracted from their environment in an additional step. One possible method of antibody-based cell selection is flow cytometry, which is known to the person skilled in the art. Such cell selection by flow cytometry is associated with significant cell loss. Therefore, purification via flow cytometry does not allow for scaling, is associated with infection risks, and therefore represents a key barrier to the commercial application of cell products. Since the methods of the invention do not require cell selection, the production of engineered skeletal muscle tissue and the cells of the invention is scalable and suitable for commercial or medical applications.

In addition, the method is serum-free, so there is no variability with respect to a different kind of serum batch. This results in a robust reproducible protocol for the production of engineered skeletal muscle tissue, in which all necessary chemical and physical stimuli are defined.

The invention further relates to a method for producing skeletal myoblasts, skeletal myotubes, and satellite cells from pluripotent stem cells, comprising the steps of
(i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
(ii) inducing myogenic specification by culturing the cells obtained in step (i) in a basal medium comprising an effective amount of (a) a gamma-secretase/NOTCH inhibitor, (b) FGF2, and (c) a serum-free additive as in (i), followed by
   continuing the cultivation in the medium with the addition of an effective amount of (d) HGF, followed by
   culturing the cells in a basal medium comprising an effective amount of (a) a gamma-secretase/NOTCH inhibitor, (b) HGF, (c) a serum-free additive as in (i), and (d) knockout serum replacement (KSR);
(iii) maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR), followed by
(iv) maturing the cells into skeletal myotubes and satellite cells by culturing the cells obtained in step (iii) in a basal medium comprising an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3) thereby generating skeletal myoblasts, skeletal myotubes, and satellite cells.

For example, the cells generated in this method have a proportion of skeletal myoblasts of the amount of all available cells of at least 40%, preferably at least 50%, more preferably at least 60%, most preferably at least 70%, as determined by expression of actinin by flow cytometry.

Preferably, the method achieves a proportion of satellite cells of the amount of all available cells of at least 10%, preferably at least 15%, more preferably at least 20%, most preferably at least 30%, determined by expression of Pax7 by flow cytometry.

The method of “flow cytometry” is known to a skilled person. In flow cytometry, physical and/or chemical properties of a cell population are detected. For the invention described herein, the presence of skeletal muscle-specific proteins characteristic of differentiation into skeletal myoblasts, skeletal myotubes, or satellite cells can be detected using fluorescent staining. Specifically, the proteins sarcomeric α-actinin, myogenin, Pax7, and MyoD are incubated with primary antibodies and thereby labelled. With the aid of a fluorescently labelled secondary antibody, the skeletal muscle-specific cells can be detected.

A major advantage of the method over the prior art is that it does not require a step of enriching cells, such as skeletal myoblasts. Preferably, the method does not contain an enrichment step by cell selection, more preferably no enrichment step by antibody-based cell selection, such as e.g. flow cytometry. This means that the method according to the invention does not require cell selection to achieve skeletal myoblasts, skeletal myotubes and/or satellite cells in high purity. Methods for cell selection are disclosed herein for analytical purposes only, to demonstrate the high purity of the skeletal myoblasts, skeletal myotubes and satellite cells produced. (See FIG. 5).

As described above, the basal medium of step (i) comprises an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof.

For example, the GSK3 inhibitor in the basal medium is selected from the group consisting of CHIR99021, CHIR98014, SB216763, TWS119, tideglusib, SB415286, 6-bromoindirubin-3-oxime, and a valproate salt, wherein the GSK3 inhibitor CHIR99021 is preferred. However, any GSK3 inhibitor suitable for the method of the invention may be used. When the GSK3 inhibitor is CHIR99021, an effective amount is 4-18 μM, preferably 5-16 μM, more preferably 6-15 μM, even more preferably 7-14 μM, even more preferably 8-13 μM, even more preferably 9-12 μM, even more preferably 9.5-11 μM, and most preferably about 10 μM.

Preferred and exemplary embodiments of steps (i)-(iii) are described in the method for preparing engineered skeletal muscle tissue and can be applied analogously to the method for preparing skeletal myoblasts, skeletal myotubes and satellite cells.

The respective stages of differentiation can be determined by simple experimental evidence known to those skilled in the art. For example, the inventors have analyzed the cells using fluorescence microscopy. This involves immunostaining of skeletal muscle-specific transcription factors (Pax7, MyoD, and myogenin). After step (iii) of the method, the fluorescence images show a high percentage of cells expressing Pax7, MyoD, and myogenin (FIG. 3). This method shows that satellite cells (Pax7) as well as skeletal myoblasts (MyoD and Myogenin) are generated by the method.

In step (iv) of the method of the invention, the cells are matured into skeletal myotubes and satellite cells. Skeletal myotubes arise by the fusion of skeletal myoblasts. Therefore, skeletal myotubes are multinucleated cellular structures formed by a fusion of mature myoblasts into long myotubes. As described in step (iv) of the method for producing skeletal muscle tissue, this differentiation stage is also characterized by the expression of specific factors. For example, the expression of Pax7 is characteristic of the presence of satellite cells. At the same time, the expression of myogenin and actinin is characteristic of skeletal myotubes, the respective expression of which can be determined by RNA sequencing (see FIGS. 1 and 2 for a schematic overview; see FIG. 4 for experimental data on the expression of PAX7, ACTN2, DMD, and MYH3). Satellite cells are given if, for example, the gene marker PAX7 has at least a 5-fold higher expression value compared with the pluripotent stem cell (preferably at least a 10-fold higher expression value, more preferably a 20-fold higher expression value) as measured by “reads per kilobase million” using RNA sequencing. Skeletal myotubes are given if, for example, the gene marker ACTN2 has an expression value at least 5-fold higher compared to the pluripotent stem cell (preferably an expression value at least 50-fold higher, more preferably an expression value 100-fold higher, even more preferably an expression value 150-fold higher), measured by “reads per kilobase million” by RNA sequencing. The gene markers DMD and MYH3 even exhibit an expression value at least 200-fold higher compared to the pluripotent stem cell (preferably an expression value at least 500-fold higher, more preferably an expression value 1000-fold higher).

As set forth above, the basal medium of step (iv) comprises an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3).

The additional serum-free additive in step (iv) of the method is formulated such that the additional serum-free additive provides a final concentration of the following substances: 0.5-50 mg/ml albumin (preferably 1-40 mg/ml, more preferably 2-30 mg/ml, even more preferably 3-20 mg/ml, even more preferably 4-10 mg/ml, and most preferably 4.5-7.5 mg/ml, such as about 5 mg/ml);

1-100 μg/ml transferrin (preferably 2-90 μg/ml, more preferably 3-80 μg/ml, even more preferably 4-70 μg/ml, even more preferably 5-60 μg/ml, more preferably 6-50 μg/ml, more preferably 7-40 μg/ml, more preferably 8-30 μg/ml, more preferably 9-20 μg/ml, such as about 10 μg/ml);

0.1-10 μg/ml ethanolamine (preferably 0.2-9 μg/ml, more preferably 0.3-8 μg/ml, even more preferably 0.4-7 μg/ml, even more preferably 0.5-6 μg/ml, more preferably 0.6-5 μg/ml, more preferably 0.7-4 μg/ml, more preferably 0.8-3 μg/ml, most preferably 1-2.5 μg/ml, such as about 2 μg/ml);

17.4-1744 nM selenium or a bioavailable salt thereof (preferably 35-850 nM, more preferably 70-420 nM, even more preferably 120-220 μg/ml, most preferably about 174 nM);

0.4-40 μg/ml L-carnitine HCl (preferably 0.5-30 μg/ml, more preferably 1-20 μg/ml, even more preferably 2-10 μg/ml, more preferably 3-5 μg/ml, and most preferably about 4 μg/ml);

0.05-5 μl/ml fatty acid additive (preferably 0.1-4 μl/ml, more preferably 0.2-3 μl/ml, even more preferably 0.3-3 μl/ml, more preferably 0.4-2 μl/ml, and most preferably 0.45-1 μl/ml, such as about 0.5 μl/ml); and

0.0001-0.1 μg/ml triiodo-L-thyronine (T3) (preferably 0.001-0.01 μg/ml, more preferably 0.002-0.0075 μg/ml, even more preferably 0.003-0.005 μg/ml, most preferably about 0.004 μg/ml).

In a preferred embodiment, the additional serum-free additive further comprises 0.1-10 μg/ml hydrocortisone (preferably 0.2-9 μg/ml, more preferably 0.3-8 μg/ml, even more preferably 0.4-7 μg/ml, even more preferably 0.5-6 μg/ml, even more preferably 0.6-5 μg/ml, even more preferably 0.7-4 μg/ml, even more preferably 0.8-3 μg/ml, most preferably 0.9-2 μg/ml, such as about 1 μg/ml). In a likewise preferred embodiment, the additional serum-free additive further comprises 0.3-30 μg/ml insulin (preferably 0.5-25 μg/ml, more preferably 1-20 μg/ml, even more preferably 1.5-15 μg/ml, even more preferably 2-10 μg/ml, most preferably 2.5-5 μg/ml, such as about 3 μg/ml). For example, a bioavailable salt of selenium is sodium selenite, such that a final concentration of 0.003-0.3 μg/ml (preferably 0.005-0.2 μg/ml, more preferably 0.01-0.1 μg/ml, even more preferably 0.02-0.05 μg/ml, and most preferably 0.03 μg/ml, such as about 0.032 μg/ml) is provided in the basal medium.

Furthermore, the additional serum-free additive may further comprise one or more components selected from the group consisting of vitamin A, hydrocortisone, D-galactose, linolenic acid, progesterone, and putrescine. These components are beneficial for cell viability. Suitable concentrations of the respective components are known to those skilled in the art or can be readily determined by routine measures. The additional serum-free additive mentioned in step (iv) is also commercially available. For example, B27 may be used. In a preferred embodiment, the B27 additive is used in an amount of 0.1-10% B27, preferably 0.5-8%, preferably 1-6%, more preferably 1.5-4%, even more preferably 1.5-4%, and most preferably about 2% B27.

In addition, step (iv) of this method may be carried out for at least 30 days, preferably at least 35 days, more preferably at least 40 days, and even more preferably at least to 50 days.

Step (i) of this method may be preceded by a seeding step, in which the pluripotent stem cells are seeded in a stem cell medium in the presence of a ROCK inhibitor, preferably wherein the seeding step is performed 18-30 hours prior to step (i), preferably 20-28 hours, more preferably 22-26 hours, even more preferably 23-25 hours, and most preferably about 24 hours. For example, the ROCK inhibitor is selected from the group consisting of Y27632, H-1152P, thiazovivin, fasudil, hydroxyfasudil, GSK429286A and RKI1447, more preferably the ROCK inhibitor is selected from the group consisting of Y27632, H-1152P, thiazovivin, fasudil, and hydroxyfasudil, more preferably the ROCK inhibitor is selected from the group consisting of Y27632 and H-1152P, wherein particularly preferably the ROCK inhibitor is Y27632. However, any ROCK inhibitor suitable for the method of the invention may be used. A person skilled in the art understands that the concentration of an effective amount of a ROCK inhibitor varies with the availability and inhibition constant of the inhibitor in question. For example, in the case of Y27632, the medium used in the seeding step may be used in a concentration of 0.5-10 μM, preferably 1-9 μM, more preferably 2-8 μM, more preferably 3-7 μM, more preferably 4-6 μM, and most preferably about 5 μM. A stem cell medium may be used in the seeding step, and in principle any stem cell medium suitable for the method may be used. Suitable stem cell media are known to those skilled in the art, with the iPS-Brew XF stem cell medium being particularly preferred. Preferably, the stem cell medium comprises an effective concentration of KSR and FGF2. In particular, the stem cell medium comprises, for example, 5-20% (v/v), preferably 6-17.5% (v/v), more preferably 7-15% (v/v), more preferably 8%-12% (v/v), more preferably 9%-11% (v/v), and most preferably about 10% (v/v) KSR; and/or 1-15 ng/ml FGF2, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml FGF2.

As in the method for producing skeletal muscle tissue, the differentiation stages of steps (i)-(iv) of the method for producing skeletal myoblasts, skeletal myotubes, and satellite cells can be detected using expressed genes that are characteristic of a particular stage. The method of RNA sequencing is used in this method analogously to the method for producing skeletal muscle tissue. Accordingly, the same expressed genes (i.e. MSGN1, TBX6, MEOX1, PAX3, PAX7, MYOD1, ACTN2, DMD, MYH3) can be detected depending on the differentiation stage.

The traditional methods disclosed in the prior art for obtaining skeletal muscle cells often require extensive digestion protocols and/or cell selection steps by flow cytometry. Digestion protocols transfer cells to a different environment, causing them to lose cell-cell connectivity as well as cell-matrix connectivity. This destroys the extracellular environment and spatial distribution of cell types formed during development, and may have an inhibitory effect on the skeletal muscle differentiation process that is difficult to control. The present invention minimizes the number of digestion steps and does not require cell selection to enrich, for example, skeletal myoblasts, as the sophisticated protocol produces a high purity of skeletal myoblasts, skeletal myotubes, and satellite cells (exemplary cell populations are shown in the examples that are at least 70% actinin positive and at least 30% PAX7 positive, see also FIG. 5).

By the method according to the invention, a skeletal muscle tissue produced in an engineered manner with advantageous properties can be obtained. In the skeletal muscle tissue produced in an engineered manner, the presence of skeletal myotubes can be detected by staining of actinin (see FIG. 8). In particular, the skeletal muscle tissue does not comprise a differentiation- or maturation-related transgene, preferably wherein the skeletal muscle tissue does not comprise a myogenic transgene, more preferably wherein the skeletal muscle tissue does not comprise the transgene Pax7 or MyoD. Compared to natural skeletal muscle tissue, the engineered skeletal muscle tissue, e.g. the BSM or ESM, does not have a blood supply or central nervous system control. The central nervous system is known to the skilled person, and consists of brain and spinal cord in vertebrates. For example, the engineered skeletal muscle tissue also has no innervation by nerve cells. Blood supply means a vascularization of the muscle, which supplies the muscle with blood. Another difference from natural skeletal muscle tissue is that engineered skeletal muscle tissue has no musculoskeletal attachment via tendon portions or bone, and develops entirely ex vivo into an engineered skeletal muscle. Thus, the engineered skeletal muscle tissue is clearly distinguishable from the natural skeletal muscle tissue. Despite its engineered production, the skeletal muscle tissue according to the invention exhibits many characteristics of a native skeletal muscle. These include morphological features of a syncytium of fused myocytes (muscle fibers, multinucleated skeletal myotubes) and contractile performance (positive force-frequency ratio and tetanic contractions). Unlike prior art tissues, satellite cell niches form in direct contact with skeletal muscle fibers. In addition, the skeletal muscle tissue produced according to the invention exhibits the typical fibers of striated skeletal muscle, with the skeletal muscle tissue consisting of many muscle fibers (syncytia). It is known to the skilled person that natural skeletal muscle tissue has multinuclear skeletal muscle fibers, with each skeletal muscle fiber consisting of sarcomeres strung together. Therefore, multinuclear skeletal muscle fibers can be recognized by their characteristic striated skeletal muscle pattern in actin staining or actinin staining, because the actin/actinin is stained within the sarcomeres. The sarcomeres have a strict, regular structure; they are arranged in a row and together form a multinuclear muscle fiber. This means that a characteristic striated skeletal muscle pattern proves that multinuclear skeletal muscle fibers have formed. The characteristic skeletal muscle tissue structure (skeletal muscle fibers with satellite cell niches) may be visualized by fluorescence microscopy after staining for actinin or actin and Pax7. In the present invention, the inventors have stained the structural protein actin in the engineered skeletal muscle tissue, and the fluorescence images in FIG. 8 exemplify the characteristic stripe pattern.

A key functional characteristic for engineered skeletal muscle tissue is that the tissue contracts in response to electrical stimulation, so that it is force generating. This force-generating character can be determined, for example, by measuring the contractile output. These contraction experiments measure the contraction frequency and contraction force of the engineered skeletal muscle tissue in response to electrical stimulation. The skeletal muscle tissue in the form of a ring was tested in organ baths (Fohr Medical Instruments) containing Tyrode's solution (e.g., in mmol/L: 120 NaCl, 1 MgCl₂, 1.8 CaCl₂), 5.4 KCl, 22.6 NaHCO₃, 4.2 NaH₂PO4, 5.6 glucose, and 0.56 ascorbate) at 37° C. and continuous gassing with 5% CO₂ and 95% O₂. The engineered skeletal muscle tissue is mechanically stretched, and the maximum force amplitude (force of contraction=FOC) is typically measured at electric field stimulation frequencies in the range of 1-100 Hz (4 ms rectangular pulses; 200 mA). Exemplary measurement methods for the tissue according to the invention are shown in FIGS. 6B, 6C, 7B, and 7C. These contraction experiments show that the engineered skeletal muscle tissue exhibits particularly advantageous properties when generating force in response to electrical stimulation. The engineered skeletal muscle tissues show a reproducible contraction frequency and contraction force in response to stimulation frequencies between 1 Hz and 100 Hz. Typically, at a single stimulation of 1 Hz, a contraction and complete relaxation takes approximately 0.5 seconds. Because the contraction and relaxation time takes approximately 0.5 seconds, incipient or complete tetanus forms at higher stimulation frequencies. Tetanus is also formed in natural skeletal muscle tissues at increased stimulation frequencies, so that the engineered skeletal muscle tissue behaves analogously to natural skeletal muscle tissue even in this regard. Furthermore, the inventors are able to show that the contraction force of the muscle tissue increases with increasing contraction frequency (positive force-frequency relationship). These properties are consistent with native skeletal muscle tissue, which also exhibits single contractions and tetanic contractions, as well as a positive force-frequency relationship in response to electrical stimulation. Unlike the engineered skeletal muscle tissue, the electrical impulses in natural muscle tissues arise from the action potentials of neurons, whereas the engineered skeletal muscle tissue can contract spontaneously and in response to electrical stimulation.

As exemplified in FIGS. 6B, 6C, 7B, 7C, and 8, the engineered skeletal muscle tissue produced by the method of the invention has a characteristic formation of multinuclear muscle fibers (skeletal myotubes) and generates force in response to electrical stimulation. Typically, the engineered skeletal muscle tissue can generate at least a contraction force of 0.3 millinewtons (mN) upon a stimulus of 100 Hz at 200 mA, preferably at least 0.4 mN, more preferably at least 0.5 mN, more preferably at least 0.6 mN, more preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, and most preferably at least 2 mN.

In principle, the engineered skeletal muscle tissue can have any desired form. For example, it may have the engineered form of a ring, a ribbon, a strand, a patch, a pouch, or a cylinder, wherein optionally individual skeletal muscle tissues are fused. For example, in the examples herein, the skeletal muscle tissue had the form of a ring. However, individual and/or different geometries may also be fused into a skeletal muscle tissue as desired, thereby achieving many other different muscle forms. Especially the form of a ring, a strand, a patch or a band is useful for applications in in vitro methods e.g. for testing the toxicity or for a therapeutic application for muscle repair in vivo. Usually, the engineered form is already obtained by casting the master mix, so that generally any castable engineered form can be produced.

Furthermore, the invention comprises mesodermally differentiated skeletal myoblast progenitor cells obtained according to step (i) of the invention, characterized by the expression of the MSGN1 and/or TBX6 genes, wherein the expression of MSGN1 and/or TBX6 can be determined by flow cytometry and/or immunostainings. These cells are also characterized in that they express the mRNA of SP5, wherein the expression of SP5 can be determined by RNA sequencing.

In addition, the invention relates to myogenically specified skeletal myoblast progenitor cells obtained according to step (ii) of the invention, produced by steps (i) and (ii) of the invention, characterized by expression of the gene PAX3, wherein the expression of PAX3 can be determined by flow cytometry and/or immunostainings. These cells are characterized in that they express the mRNA of SIM1, wherein the expression of SIM1 can be determined by RNA sequencing.

Furthermore, the invention relates to skeletal myoblast cells obtained according to step (iii) of the invention, produced by steps (i) to (iii) of the invention, characterized by the expression of actinin, wherein the expression of actinin can be determined by flow cytometry and/or immunostainings in skeletal myoblasts.

Further provided by the present disclosure are satellite cells, obtainable according to step (iii) of the methods disclosed herein and producible by steps (i) through (iii) of the methods disclosed herein, which are characterized by the expression of the gene Pax7. In this regard, the expression of Pax7 can be determined by flow cytometry and/or immunostainings. Satellite cells are characterized by an active or activatable cell cycle and then express Pax7 and Ki67. In a particularly preferred embodiment, the satellite cells therefore further express Ki67. The cell cycle activation in engineered skeletal muscle tissue is observed more frequently after tissue damage (e.g., by pressure injury, cardiotoxin treatment, irradiation, or frostbite) and leads to a repair of the tissue damage in the sense of an endogenous regeneration.

Also disclosed is a mixture of skeletal myoblast cells and satellite cells, wherein a proportion of satellite cells of the amount of all available cells is achieved of at least 10%, preferably at least 15%, more preferably at least 20%, even more preferably at least 30%, determined by expression of Pax7 by flow cytometry; and/or wherein a proportion of skeletal myoblasts of the amount of all available cells is obtained of at least 40%, preferably at least 50%, more preferably at least 60%, most preferably at least 70%, determined by the expression of actinin by flow cytometry.

Furthermore, the invention relates to skeletal myotubes obtained according to step (iv) of the invention, produced by steps (i) to (iv) of the invention, characterized by an anisotropic orientation of the actinin protein-containing sarcomere structure. Advantageously, the engineered skeletal muscle tissue, the mesodermally differentiated skeletal myoblast precursor cells, the myogenically specified skeletal myoblast precursor cells, the skeletal myoblast cells, the satellite cells, and/or the skeletal myotubes may be used in an in vitro drug assay. The drug assay is preferably a toxicity assay or an assay for skeletal muscle tissue function under the influence of pharmacological and gene therapeutic drug candidates. Pharmacological drug candidates are typically drug candidates that comprise small molecule compounds as well as protein-based molecules. Gene therapeutic drug candidates typically alter the genome of the skeletal muscle tissue by introducing corresponding nucleic acids.

Furthermore, the engineered skeletal muscle tissue, mesodermally differentiated skeletal myoblast progenitor cells, myogenically specified skeletal myoblast progenitor cells, skeletal myoblast cells, satellite cells, and/or skeletal myotubes can be used in medicine.

Of particular importance here are the satellite cells. They are contemplated for use in the therapy of damaged skeletal muscle and/or in the treatment of skeletal muscle diseases, preferably in genetic skeletal muscle defects, in particular Duchenne muscular dystrophy and/or Becker-Kiener muscular dystrophy, and/or lysosomal storage diseases, in particular Pompe disease, preferably wherein the skeletal muscle disease is Duchenne muscular dystrophy. The skilled person is aware from the prior art that satellite cells have already been applied in clinical studies in the therapy of muscular dystrophies (Tedesco F S et al. 2010). In addition, satellite cells are being considered for use in the treatment of skeletal muscle diseases, such as amyotrophic lateral sclerosis, mysthenia gravis, or myotonia. Myotonia subsumes various muscle diseases that exhibit delayed relaxation and consequently pathologically prolonged tonic muscle contraction. Satellite cells are particularly suitable for the therapy of damaged skeletal muscle and/or for the treatment of skeletal muscle diseases, as they continuously regenerate the skeletal muscle tissue. The term “damaged skeletal muscle tissue” refers to injuries and wounding of a tissue caused by external force. Human satellite cells obtained according to step (iii) or (iv) of the method of the invention exhibit the characteristic marker Pax7. Therefore, satellite cells according to the invention are promising candidates for cell-based therapy in damaged skeletal muscle tissue, as satellite cells lead to increased regeneration of skeletal muscle tissue (Yin et al. (2013)). Similarly, engineered skeletal muscle tissues according to the invention are promising candidates for cell-based therapy in damaged skeletal muscle tissue; especially for the treatment of large muscle defects. Especially with trauma or massive muscle destruction, a direct implantation of replacement tissue, such as engineered skeletal muscle tissue, is a promising approach. Skeletal muscle implants can be functionally integrated and controllable via electrical stimulation or optogenetic activation, so as to restore or therapeutically support muscle function. The satellite cell proportion in engineered skeletal muscle tissue ensures the endogenous regenerative capacity of skeletal muscle in the long term.

The engineered skeletal muscle tissue, as well as the mesodermally differentiated skeletal myoblast progenitor cells, myogenically specified skeletal myoblast progenitor cells, satellite cells, skeletal myoblast cells, skeletal myotubes, or a mixture of skeletal myoblasts and satellite cells, are also suitable model systems for studying cellular mechanisms important for differentiation and maturation. Thus, they represent important scientific tools for basic research. Thus, for example, chemical substances and, optionally, physical stimuli such as stretching or damage can be tested outside the human body on human cells or on engineered skeletal muscle tissue. The cells, skeletal myotubes and engineered skeletal muscle tissue according to the invention enable pharmacological safety and efficacy experiments, whereby the effect on cells and tissue can be tested. This is a clear advantage over animal experiments (e.g., mouse or rat tissues/cells), as the pharmacological effect can be tested on e.g. human tissues and, in a particular embodiment, patient-specific tissues. Due to the high similarity to natural skeletal tissue, the skeletal tissue produced according to the above disclosed method can advantageously be used in various in vitro procedures.

One such possible application is an in vitro method for testing the efficacy of a drug candidate on a skeletal muscle tissue, comprising the steps of
(a) providing a skeletal muscle tissue in accordance with the invention described herein,
(b) optionally inflicting damage on the skeletal muscle tissue, and
(c) contacting the skeletal muscle tissue of step (a) or (b) with a drug candidate; preferably wherein the method further comprises determining the contraction force and/or the structure of the skeletal muscle tissue and/or the metabolic function and/or molecular and/or protein biochemical parameters before and/or after step (c).

The contraction force and/or structure of the skeletal muscle tissue may be measured by the contraction experiments described herein and by the fluorescence microscopy experiments described herein. For example, metabolic function can be measured by using a Seahorse Metabolic Flux Analyzer, which is known to those skilled in the art. For example, a Seahorse Metabolic Flux Analyzer measures the oxygen consumption and extracellular acid generation rate of living cells and can further measure important cellular functions such as mitochondrial respiration and glycolysis. Molecular parameters (markers) can be measured, for example, by transcriptome analyses (PCR or RNA sequencing). Protein biochemical parameters (markers) can be measured, for example, via mass spectrometry or common clinical chemistry measurement methods (e.g., ELISA or other antibody and/or chromatographic and/or electrophoretic and/or affinity-based methods). These molecular and protein biochemical parameters are also called markers or biomarkers, and common biomarkers related to skeletal muscle are known to those skilled in the art. For example, creatine kinase (also known as creatine kinase CK, CPK, or creatine phosphokinase) and L-lactate dehydrogenase (LDH) are such biomarkers.

Drug candidates comprise pharmacological drug candidates such as drug candidates comprising small molecule compounds and protein-based or nucleic acid-based molecules. Furthermore, drug candidates include gene therapeutic drug candidates, which typically modify the genome of cells of the invention by introducing corresponding nucleic acids. In addition, drug candidates can also be a body's own substances, so that the effect of, for example, hormones or hormone-like signal substances can be tested. Examples of hormone-like signalling substances are myokines, such as myostatin, follistatin, irisin, visfatin and myonectin.

A further in vitro method is contemplated for testing the toxicity of a substance on a skeletal muscle tissue, comprising the steps of
(a) providing a skeletal muscle tissue according to the invention described herein,
(b) contacting the skeletal muscle tissue from step (a) with a substance to be tested, preferably wherein the method further comprises determining the contraction force and/or structure of the skeletal muscle tissue and/or metabolic function and/or molecular and/or protein biochemical parameters before and/or after step (b).

The contraction force and/or skeletal muscle tissue structure may be measured by the contraction experiments described herein and by the fluorescence microscopy experiments described herein. The metabolic function may be measured, for example, by using a Seahorse Metabolic Flux Analyzer, which is known to those skilled in the art.

The substances used in toxicity testing may be, but are not limited to, drug candidates, for example. Rather, any substance whose toxicity is to be evaluated can be tested.

Other possible applications relate to an in vitro method for testing the effect of nutrients and dietary supplements on skeletal muscle tissue performance, comprising the steps of
(a) providing a skeletal muscle tissue in accordance with the invention described herein,
(b) contacting the skeletal muscle tissue from step (a) with a nutrient and nutritional supplement to be tested,
preferably wherein the method further comprises determining the contraction force and/or structure of the skeletal muscle tissue and/or metabolic function and/or molecular and/or protein biochemical parameters before and/or after step (b).

This in vitro method provides the opportunity to measure the effects of nutrients and dietary supplements on skeletal muscle tissue at clinically relevant concentrations. This method is of particular interest when measuring the effects of these substances in muscle growth, cachexia, or diabetes mellitus. Cachexia is understood to be a pathological, very severe emaciation. Many patients with chronic diseases such as cancer or autoimmune diseases suffer from the additional condition of cachexia. The in vitro method offers the possibility to measure the effect of substances on skeletal muscle tissue outside the body.

However, similarly, the various cells prepared according to the method disclosed herein can also be used in such in vitro methods. For example, an in vitro method for testing the efficacy of a drug candidate on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells is described herein, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the invention described herein,
(b) optionally inflicting damage on the cells from step (a), and
(c) contacting the cells of step (a) or (b) with a drug candidate;
preferably wherein the method further comprises determining the expression of actinin and/or Pax7 before and/or after step (c), wherein the expression can be determined by flow cytometry and/or immunostainings.

Another possible application relates to an in vitro method for testing the toxicity of a substance on mesodermally differentiated skeletal myoblast progenitor cells, myogenically specified skeletal myoblast progenitor cells, satellite cells, skeletal myoblast cells, skeletal myotubes or a mixture of skeletal myoblast cells and satellite cells, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the invention described herein,
(b) contacting the cells of step (a) with a substance to be tested,
preferably wherein the method further comprises determining the expression of actinin and/or Pax7 before and/or after step (b), wherein the expression can be determined by flow cytometry and/or immunostainings.

An additional possible application relates to an in vitro method for testing the effect of nutrients and dietary supplements on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
(a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to the invention described herein,
(b) contacting the cells of step (a) with a nutrient or dietary supplement to be tested,
preferably wherein the method further comprises determining the expression of actinin and/or Pax7 before and/or after step (b), wherein the expression can be determined by flow cytometry and/or immunostainings.

In another preferred embodiment, the skeletal muscle tissue can generate a contraction force of at least 0.6 millinewtons (mN) upon a stimulus of 100 Hz, preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, and most preferably at least 2 mN are generated. The contraction force is typically measured above the stimulus threshold. Suitable methods for determining a stimulus threshold are known to those skilled in the art. For example, the contraction force can be recorded at an electric field stimulation with 200 mA (see FIGS. 6, 7, 9 and 10). In a more preferred embodiment, skeletal muscle tissue can generate at least a contraction force of 2 millinewtons (mN) upon a stimulus of 100 Hz, preferably at least 2.3 mN, more preferably at least 2.6 mN, even more preferably at least 3 mM, even more preferably at least 3.3 mN, even more preferably at least 3.6 mN, most preferably at least 4 mN. This typically occurs when step (iv) of the method is carried out for at least 50 days, such as e.g. 56 days. A typical characteristic of the engineered skeletal muscle tissue described herein is that the contraction force increases with the duration of the maturation.

In another preferred embodiment, the skeletal muscle tissue has a contraction speed of at least 3 mN/sec upon a stimulation of 100 Hz, preferably at least 4 mN/sec, more preferably at least 5 mN, more preferably at least 6 mN/sec, even more preferably at least 6.5 mN/sec, even more preferably at least 7 mN/sec. For example, the contraction speed can be recorded at a stimulation of 100 Hz with 200 mA (5 ms, mono- or biphasic). The contraction speed, also called force generation speed, is the time required for an engineered skeletal muscle tissue to build up an amount of tension, or the rate of tension increase, respectively. The contraction speed is determined as the time point of the maximum increase in contraction force (+dFOC/dt) in the context of an isometric contraction experiment.

In another preferred embodiment, the skeletal muscle tissue has a relaxation speed of at least 0.5 mN/sec upon termination of a stimulation of 100 Hz, preferably at least 0.7 mN/sec, more preferably at least 0.9 mN/sec more preferably at least 1 mN/sec, even more preferably at least 1.2 mN/sec, even more preferably at least 1.5 mN/sec. The relaxation speed is determined in the relaxation phase of the skeletal muscle as the time point of the maximum decrease of the contraction force (−dFOC/dt) in the context of an isometric contraction experiment.

In a particularly preferred embodiment, the basal medium in step (iv) may comprise an effective amount of creatine and/or triiodo-L-thyronine (T3). For example, if creatine is present in an effective amount in the basal medium of the maturation medium, the contraction force of the engineered skeletal muscle may increase compared to a maturation in step (iv) without an effective amount of creatine. Such an increase in contraction force is shown in Example 4 and FIG. 9 with experimental data. For example, an effective amount of creatine as a final concentration in the basal medium of step (iv) is 0.1-10 mM creatine. More preferred concentrations are, for example, 0.2-6 mM creatine, more preferred 0.4-4 mM creatine, even more preferred 0.6-3 mM creatine, even more preferred 0.7-2.5 mM creatine, even more preferred 0.8-2 mM creatine, even more preferred 0.85-1.5 mM creatine, even more preferred 0.9-1.2 mM creatine, and most preferred about 1 mM creatine.

Furthermore, the maturation medium in step (iv) may also have an increased amount of T3. Such an increased amount of T3 may reduce the contraction speed and/or relaxation speed of the engineered skeletal muscle compared to an engineered skeletal muscle tissue prepared without an increased amount of T3 in step (iv). Exemplary increased amounts of T3 in the basal medium in step (iv) are 0.001-1 μM triiodo-L-thyronine (T3), preferably 0.005-0.7 μM T3, more preferably 0.01-0.35 μM T3, even more preferably 0.04-0.02 μM T3, even more preferred 0.05-0.18 μM T3, even more preferred 0.06-0.15 μM T3, even more preferred 0.08-0.12 μM T3, even more preferred about 0.1 μM T3. Furthermore, Example 4 as well as FIG. 10 shows the advantageous effect with experimental data of an increased concentration of T3.

In a particularly strongly preferred embodiment, the basal medium in step (iv) may comprise an effective amount of creatine and/or an increased amount of triiodo-L-thyronine (T3) for a given time period of the maturation. As shown in Example 4, such a time period may be 4 weeks, for example, from week 1 to week 5 in step (iv), or week 5 to week 9 in step (iv). However, other time periods, such as 1-9 weeks, may be selected during any maturation period. For example, this time period may be at least one week, preferably at least 2 weeks, more preferably at least 3 weeks, more preferably at least 4 weeks, even more preferably at least 5 weeks, even more preferably at least 6 weeks, even more preferably at least 7 weeks, even more preferably at least 8 weeks. Further, this time period may be, for example, at most 9 weeks, more preferably at most 8 weeks, more preferably at most 7 weeks, even more preferably at most 6 weeks, even more preferably at most 5 weeks, even more preferably at most 4 weeks. In light of the present disclosure, the person skilled in the art may freely combine the exemplary time period endpoints.

In another preferred embodiment, the skeletal muscle tissue produced by the method described herein has a regenerative property. The regenerative property is characterized by bringing about a natural restoration of the previously existing condition. For example, the contractility of an engineered skeletal muscle tissue can be restored. Thus, a contractility can be regained and/or the muscle can be rebuilt. In a strongly preferred embodiment, the regenerative property is characterized by a regained contractility and/or muscle reconstruction, preferably wherein the ability to regain contractility and/or muscle reconstruction is measured after a 24-hour exposure to cardiotoxin and/or muscle reconstruction, more preferably wherein said regained contractility and/or muscle reconstruction is measured 10-30 days after a cardiotoxin exposure. Cardiotoxin is a polypeptide toxin and destroys skeletal muscle cells by inducing permanent depolarization. Functionally, incubation with cardiotoxin results in a loss of contractility of engineered skeletal muscle. Structurally, irreversible destruction of formed myotubes in engineered skeletal muscle is observed. Even after, for example, 2 days, no contractions could be recorded in Example 5 described here. As shown in FIG. 11, an engineered skeletal muscle tissue with regenerative property can regain this contractility. For example, as described in Example 5, a muscle can contract again 21 days after cardiotoxin treatment. However, engineered skeletal muscle treated with gamma radiation (X-rays) does not exhibit regenerative property and cannot contract even 21 days after cardiotoxin incubation, for example. This example shows that in engineered skeletal muscle tissue, when skeletal muscle cells are irreversibly destroyed, skeletal muscle cell precursor cells with regenerative capacity are preserved and can regenerate or rebuild the skeletal muscle structure with contractile function in engineered skeletal muscle tissue by cell division and differentiation into newly formed skeletal muscle cells. As shown in FIG. 11, engineered skeletal muscle tissue with regenerative property can accomplish this muscle reconstruction. FIG. 11B shows the regeneration of contraction force, and FIG. 11C (top) shows the structural regeneration of skeletal muscle. Failure of regeneration after gamma irradiation demonstrates that skeletal muscle progenitor cells (e.g., satellite cells) competent for cell division for regeneration survived the cardiotoxin treatment.

As shown in Example 4, step (iv) of the procedure can be extended over several weeks. In a strongly preferred embodiment, step (iv) is performed for at least 50 days, more preferably at least 60 days, even more preferably at least 70 days, even more preferably at least 80 days. To the present knowledge of the inventors, there is no upper limit to the duration of step (iv). For example, a maximum duration of step (iv) may be 365 days, preferably 300 days, more preferably 250 days. The person skilled in the art may freely combine the exemplary time period limits of step (iv) in light of the present disclosure.

Furthermore, the present invention comprises an engineered skeletal muscle tissue produced by a method as described herein. In a preferred embodiment, the skeletal muscle tissue generates upon a stimulus of 100 Hz at least a contraction force of 0.6 millinewtons (mN), preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, more preferably at least 2 mN, more preferably at least 2.3 mN, more preferably at least 2.6 mN, even more preferably at least 3 mM, even more preferably at least 3.3 mN, even more preferably at least 3.6 mN, and most preferably at least 4 mN. The contraction force can be recorded, for example, at a stimulation of 200 mA.

In a particularly preferred embodiment, the skeletal muscle tissue has a contraction speed of at least 3 mN/sec upon a stimulation of 100 Hz, preferably at least 4 mN/sec, more preferably at least 5 mN/sec, more preferably at least 6 mN/sec, even more preferably at least 6.5 mN/sec, even more preferably at least 7 mN/sec. In another preferred embodiment, the skeletal muscle tissue has a relaxation speed of at least 0.5 mN/sec when terminating a stimulation of 100 mN/sec, preferably at least 0.7 mN/sec, more preferably at least 0.9 mN/sec, more preferably at least 1 mN/sec, even more preferably at least 1.2 mN/sec, even more preferably at least 1.5 mN/sec.

The invention is further described by the following embodiments:
1. A method for producing engineered skeletal muscle tissue from pluripotent stem cells, comprising the steps of
   (i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
   (ii) inducing the myogenic specification by culturing the cells obtained in step (i) in a basal medium comprising an effective amount of (a) a gamma-secretase/NOTCH inhibitor, (b) FGF2, and (c) a serum-free additive as in (i), followed by
      continuing the cultivation in the medium with the addition of an effective amount of (d) HGF, followed by
      culturing the cells in a basal medium comprising an effective amount of (a) a gamma secretase/NOTCH inhibitor, (b) HGF, (c) a serum-free additive as in (i), and (d) knockout serum replacement (KSR);
   (iii) expanding and maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR);
   (iv) maturing the cells into skeletal myotubes and satellite cells by culturing the cells obtained in step (iii), which are dispersed in an extracellular matrix, under mechanical stimulation in a basal medium, comprising an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3);
   thereby producing engineered skeletal muscle tissue.
2. The method of embodiment 1, wherein the pluripotent stem cells are of primate origin, in particular human pluripotent stem cells; and/or wherein the pluripotent stem cells are selected from induced pluripotent stem cells, embryonic stem cells, parthenogenetic stem cells, pluripotent stem cells produced via nuclear transfer, and pluripotent cells produced via chemical reprogramming, in particular wherein the pluripotent stem cells are induced pluripotent stem cells.
3. The method of embodiment 1 or 2, wherein step (i) is carried out for 24 to 132 hours, preferably for 48 to 120 hours, more preferably for 60 to 114 hours, even more preferably for 72 to 108 hours, more preferably for 84 to 102 hours, and most preferably for about 96 hours.
4. The method of any one of embodiments 1-3, wherein in step (i), the GSK3 inhibitor is selected from the group consisting of CHIR99021, CHIR98014, SB216763, TWS119, tideglusib, SB415286, 6-bromoindirubin-3-oxime and a valproate salt, preferably wherein the GSK3 inhibitor is CHIR99021; and/or wherein in step (i), the SMAD inhibitor is selected from the group consisting of LDN193189, K02288, LDN214117, ML347, LDN212854, DMH1, preferably wherein the SMAD inhibitor is LDN193189.
5. The method of any one of embodiments 1-4, wherein in step (i), the effective amount of FGF2 is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or the serum-free additive provides in the medium a final concentration of 50-500 μg/ml transferrin, 1-20 μg/ml insulin, 0.001-0.1 μg/ml progesterone, 5-50 μg/ml putrescine and 6-600 nM selenium or a bioavailable salt thereof, in particular sodium selenite, and/or
   the GSK3 inhibitor is CHIR99021, and the effective amount is 1-20 μM, preferably 2-19 μM, more preferably 3-18 μM, even more preferably 4-17 μM, even more preferably 5-16 μM, even more preferably 6-15 μM, even more preferably 7-14 μM, even more preferably 7.5-13 μM, even more preferably 8-12 μM, even more preferably 9-11 μM, and most preferably about 10 μM; and/or
   the SMAD inhibitor is LDN193189, and the effective amount is 0.05-5 μM, preferably 0.1-2.5 μM, more preferably 0.2-1 μM, even more preferably 0.25-0.8 μM, even more preferably 0.3-0.75 μM, even more preferably 0.35-0.7 μM, even more preferably 0.4-0.6 μM, even more preferably 0.45-0.55 μM, and most preferably about 0.5 μM.
6. The method of any of embodiments 1-5, wherein the serum-free additive in step (i) is 0.1-10% (v/v) N2 additive, more preferably 0.3-7.5% (v/v) N2 additive, more preferably 0.5-5% (v/v) N2 additive, more preferably 0.75%-2% (v/v) N2 additive, more preferably 0.9%-1.2% (v/v) N2 additive, and most preferably about 1% (v/v) N2 additive.
7. The method of any of embodiments 1-6, wherein the basal medium in step (i), step (ii), step (iii), and/or in step (iv) is selected from DMEM, DMEM/F12, RPMI, IMDM, alphaMEM, Medium 199, Hams F-10, Hams F-12, wherein the basal medium is preferably DMEM, in particular wherein the basal medium is supplemented with pyruvate and/or non-essential amino acids, and/or comprises 1 g/l glucose.
8. The method of any one of embodiments 1-7, wherein in step (ii), the cultivation is carried out in the presence of (a) a gamma-secretase/NOTCH inhibitor, (b) FGF2, and (c) the serum-free additive for 36 to 60 hours, preferably for 42 to 54 hours, and most preferably for about 48 hours; and/or the cultivation is carried out in the presence of (a) a gamma-secretase/NOTCH inhibitor, (b) FGF2, (c) the serum-free additive, and (d) HGF for 36 to 60 hours, preferably for 42 to 54 hours, and most preferably for about 48 hours; and/or the culturing is carried out in the presence of (a) a gamma-secretase/NOTCH inhibitor, (b) HGF, (c) the serum-free additive, and (d) knockout serum replacement (KSR) for 72 to 120 hours, preferably for 76 to 114 hours, more preferably for 84 to 108 hours, even more preferably for 90 to 102 hours, and most preferably for about 96 hours.
9. The method of any of embodiments 1-8, wherein in step (ii) the gamma-secretase/NOTCH inhibitor is selected from the group DAPT, 804929097, semagacestat (LY450139), avagacestat (BMS-708163), dibenzazepine (YO-01027), LY411575, IMR-1, L685458, preferably wherein the gamma-secretase/NOTCH inhibitor is DAPT.
10. The method of any one of embodiments 1-9, wherein in step (ii), the effective amount of FGF2 is 15-30 ng/ml, preferably 17.5-25 ng/ml, more preferably 18-22 ng/ml, even more preferably 19-21 ng/ml, and most preferably about 20 ng/ml; and/or
   the effective amount of HGF is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or
   the gamma-secretase/NOTCH inhibitor is DAPT, and the effective amount is 1-20 μM, preferably 2-19 μM, more preferably 3-18 μM, even more preferably 4-17 μM, even more preferably 5-16 μM, even more preferably 6-15 μM, even more preferably 7-14 μM, even more preferably 7.5-13 μM, even more preferably 8-12 μM, even more preferably 9-11 μM, and most preferably about 10 μM;
   the KSR is used in an amount of 5-20% (v/v), preferably 6-17.5% (v/v), more preferably 7-15% (v/v), more preferably 8%-12% (v/v), more preferably 9%-11% (v/v), and most preferably about 10% (v/v) KSR; in particular wherein the KSR is used in the presence of a reducing agent such as beta-mercaptoethanol and/or alpha-thioglycerol.
11. The method of any one of embodiments 1-10, wherein in step (iii), the effective amount of HGF is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or the KSR is used in an amount of 5-20% (v/v), preferably 6-17.5% (v/v), more preferably 7-15% (v/v), more preferably 8%-12% (v/v), more preferably 9%-11% (v/v), and most preferably about 10% (v/v) KSR; in particular wherein the KSR is used in the presence of a reducing agent such as beta-mercaptoethanol and/or alpha-thioglycerol.
12. The method of any one of embodiments 1-11, wherein in step (iv), the additional serum-free additive provides in the medium a final concentration of 0.5-50 mg/ml albumin, 1-100 μg/ml transferrin, 0.1-10 μg/ml ethanolamine, 17.4-1744 nM selenium or a bioavailable salt thereof, in particular sodium selenite, 0.4-40 μg/ml L-carnitine, 0.05-5 μl/ml fatty acid additive, 0.0001-0.1 μg/ml triiodo-L-thyronine (T3).
13. The method of any of embodiments 1-12, wherein the additional serum-free additive in step (iv) is 0.1-10% (v/v) B27, preferably 0.5-8% (v/v), more preferably 1-6% (v/v), even more preferably 1.5-4% (v/v), and most preferably about 2% (v/v) B27.
14. The method of any of embodiments 1-13, wherein in step (iv), the mechanical stimulation is a static tension or a dynamic stimulation or an auxotonic stimulation, preferably wherein the mechanical stimulation is a static tension.
15. The method of any one of embodiments 1-14, comprising, prior to step (i), a seeding step, wherein the pluripotent stem cells are seeded in a stem cell medium in the presence of a ROCK inhibitor, preferably wherein the seeding step is carried out 18-30 hours prior to step (i).
16. The method of embodiment 15, wherein the ROCK inhibitor is selected from the group consisting of Y27632, H-1152P, thiazovivin, fasudil, hydroxyfasudil, GSK429286A and RKI1447, preferably the ROCK inhibitor is selected from the group consisting of Y27632, H-1152P, thiazovivin, fasudil and hydroxyfasudil, more preferably the ROCK inhibitor is selected from the group consisting of Y27632 and H-1152P, particularly preferably wherein the ROCK inhibitor is Y27632.
17. The method of embodiment 15 or 16, wherein the ROCK inhibitor is Y27632 and is used at a concentration of 0.5-10 μM, preferably 1-9 μM, more preferably 2-8 μM, more preferably 3-7 μM, more preferably 4-6 μM, and most preferably at a concentration of about 5 μM; and/or
   wherein the stem cell medium is iPS-Brew XF.
18. The method of any one of embodiments 15-17, wherein the pluripotent stem cells in the seeding step are first seeded into an engineered form in the presence of one or more components of an extracellular matrix in a master mix before the stem cell medium is added.
19. The method of embodiment 18, wherein the component of an extracellular matrix in the master mix is collagen, preferably type I collagen, more preferably of bovine origin, human origin or marine origin, in particular collagen of bovine origin, optionally wherein the extracellular matrix additionally comprises laminin and/or fibronectin.
20. The method of embodiment 19, wherein the pluripotent stem cells are seeded in medium at a ratio of 1-6×10⁶ cells/ml and 0.7-1.4 mg/ml collagen.
21. The method of any of embodiments 18-20, wherein the master mix comprises 5-15% (v/v) of an exudate of Engelbreth-Holm-Swarm (EHS) mouse sarcoma cells as an extracellular matrix component, preferably 7.5%-12.5% (v/v), more preferably 9-11% (v/v), and most preferably comprises about 10% (v/v), in particular wherein the exudate is Matrigel; and/or wherein the pH of the mastermix is pH 7.2 to pH 7.8.
22. The method of any of embodiments 18-20, wherein the master mix comprises stromal cells, wherein the stromal cells generate the extracellular matrix components collagens, laminin, fibronectin and/or proteoglycans; and/or wherein the pH of the master mix is pH 7.2 to pH 7.8.
23. The method of any one of embodiments 18-22, wherein the stem cell medium is added to the master mix in the engineered form after about 1 hour, wherein the stem cell medium comprises KSR and FGF2.
24. The method of embodiment 23, wherein the stem cell medium comprises 5-20% (v/v), preferably 6-17.5% (v/v), more preferably 7-15% (v/v), more preferably 8%-12% (v/v), more preferably 9%-11% (v/v), and most preferably about 10% (v/v) KSR; and/or
   wherein the stem cell medium comprises 1-15 ng/ml FGF2, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml FGF2.
25. The method of any of embodiments 18-24, wherein step (iii) is carried out for 7-11 days, preferably for 8-10 days, and most preferably for about 9 days.
26. The method of any of embodiments 1-17, wherein after step (iii), the skeletal myoblasts and satellite cells are seeded into an engineered form in an additional step prior to step (iv) in the presence of one or more components of an extracellular matrix in a master mix.
27. The method of embodiment 26, wherein the component of an extracellular matrix in the master mix is collagen, preferably type I collagen, more preferably of bovine origin, human origin or marine origin, in particular collagen of bovine origin, optionally wherein the extracellular matrix additionally comprises laminin and/or fibronectin.
28. The method of embodiment 27, wherein the skeletal myoblasts and satellite cells are seeded in medium at a ratio of 1-10×10⁶ cells/ml and 0.7-1.4 mg/ml collagen.
29. The method of any one of embodiments 26-28, wherein the master mix comprises 5-15% (v/v) of an exudate of Engelbreth-Holm-Swarm (EHS) mouse sarcoma cells as an extracellular matrix component, preferably 7.5%-12.5% (v/v), more preferably 9-11% (v/v), and most preferably about 10% (v/v), in particular wherein the exudate is Matrigel; and/or
   wherein the pH of the mastermix is pH 7.2 to pH 7.8.
30. The method of any one of embodiments 26-28, wherein the master mix comprises stromal cells, wherein the stromal cells produce the extracellular matrix components collagens, laminin, fibronectin, and/or proteoglycans; and/or
   wherein the pH of the master mix is pH 7.2 to pH 7.8.
31. The method of any one of embodiments 26-30, wherein after about one hour, a medium as used in step (iii) is added to a master mix in an engineered form, wherein the medium additionally comprises an effective amount of a ROCK inhibitor;
   in particular wherein the ROCK inhibitor is selected from the group consisting of Y27632, H-1152P, thiazovivin, fasudil, hydroxyfasudil, GSK429286A and RKI1447, preferably the ROCK inhibitor is selected from the group consisting of Y27632, H-1152P, thiazovivin, fasudil and hydroxyfasudil, more preferably the ROCK inhibitor is selected from the group consisting of Y27632 and H-1152P, particularly preferably the ROCK inhibitor is Y27632.
32. The method of embodiment 31, wherein the ROCK inhibitor is Y27632 and is used at a concentration of 0.5-10 μM, preferably 1-9 μM, more preferably 2-8 μM, more preferably 3-7 μM, more preferably 4-6 μM, and most preferably at a concentration of about 5 μM.
33. The method of any one of embodiments 26-32, wherein after about one day, the medium is replaced with a medium as used in step (iii), and the cells are then further cultured in this medium for a further 5-9 days, preferably 6-8 days, most preferably about 7 days.
34. The method of any of embodiments 18-33, wherein the engineered form has the form of a ring, ribbon, strand, patch, pouch, or cylinder, wherein optionally individual skeletal muscle tissues are fused.
35. The method of any of embodiments 1-34, wherein step (iv) is carried out for at least 19 days, preferably at least 28 days, more preferably for at least 56 days, even more preferably is carried out for at least 120 days, and in particular is carried out for at least 240 days.
36. The method of any one of embodiments 1-35, wherein the method does not comprise a differentiation- or maturation-related transgene, preferably wherein the method does not comprise a myogenic transgene, more preferably wherein the method does not comprise the transgene Pax7 or MyoD.
37. The method of any of embodiments 1-36, wherein the method does not comprise a skeletal myoblast enrichment step, preferably not an enrichment step by cell selection, more preferably not an enrichment step by antibody-based cell selection.
38. A method for producing skeletal myoblasts, skeletal myotubes, and satellite cells from pluripotent stem cells, comprising the steps of
   (i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine, and selenium or a bioavailable salt thereof;
   (ii) inducing myogenic specification by culturing the cells obtained in step (i) in a basal medium comprising an effective amount of (a) a gamma-secretase/NOTCH inhibitor, (b) FGF2, and (c) a serum-free additive as in (i), followed by
      continuing the cultivation in the medium with the addition of an effective amount of (d) HGF, followed by
      culturing the cells in a basal medium comprising an effective amount of (a) a gamma-secretase/NOTCH inhibitor, (b) HGF, (c) a serum-free additive as in (i), and (d) knockout serum replacement (KSR);
   (iii) maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR), followed by
   (iv) maturing the cells into skeletal myotubes and satellite cells by culturing the cells obtained in step (iii) in a basal medium comprising an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3)
      thereby producing skeletal myoblasts, skeletal myotubes, and satellite cells.
39. The method of embodiment 38, wherein a proportion of skeletal myoblasts of the amount of available all cells of at least 40% is achieved by the method, preferably at least 50%, more preferably at least 60%, most preferably at least 70%, as determined by the expression of actinin by flow cytometry.
40. The method of embodiment 38 or 39, wherein a proportion of satellite cells of the amount of all available cells of at least 10% is achieved by the method, preferably at least 15%, more preferably at least 20%, most preferably at least 30% is achieved, determined by expression of Pax7 by flow cytometry.
41. The method of any of embodiments 38-40, wherein the method does not include a skeletal myoblast enrichment step, preferably not an enrichment step by cell selection, more preferably not an enrichment step by antibody-based cell selection.
42. The method of any of embodiments 38-41, wherein the pluripotent stem cells are of primate origin, in particular human pluripotent stem cells; and/or wherein the pluripotent stem cells are selected from induced pluripotent stem cells, embryonic stem cells, parthenogenetic stem cells, pluripotent stem cells produced via nuclear transfer, and pluripotent cells produced via chemical reprogramming, in particular wherein the pluripotent stem cells are induced pluripotent stem cells.
43. The method of any one of embodiments 38-42, wherein step (i) is carried out for 48 to 132 hours, preferably for 48 to 120 hours, more preferably for 60 to 114 hours, even more preferably for 72 to 108 hours, more preferably for 84 to 102 hours, and most preferably for about 96 hours.
44. The method of any one of embodiments 38-43, wherein in step (i), the GSK3 inhibitor is selected from the group consisting of CHIR99021, CHIR98014, SB216763, TWS119, tideglusib, SB415286, 6-bromoindirubin-3-oxime and a valproate salt, preferably wherein the GSK3 inhibitor is CHIR99021; and/or wherein in step (i), the SMAD inhibitor is selected from the group consisting of LDN193189, K02288, LDN214117, ML347, LDN212854, DMH1, preferably wherein the SMAD inhibitor is LDN193189.
45. The method of any one of embodiments 38-44, wherein in step (i), the effective amount of FGF2 is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or
   the serum-free additive provides a final concentration of 50-500 mg/I transferrin, 1-20 mg/I insulin, 1-30 μg/l progesterone, 5-50 μg/ml putrescine and 6-600 nM selenium or a bioavailable salt thereof, in particular sodium selenite, in the medium, and/or
   the GSK3 inhibitor is CHIR99021, and the effective amount is 4-18 μM, preferably 5-16 μM, more preferably 6-15 μM, even more preferably 7-14 μM, even more preferably 8-13 μM, even more preferably 9-12 μM, even more preferably 9.5-11 μM, and most preferably about 10 μM; and/or
   the SMAD inhibitor is LDN193189, and the effective amount is 0.05-5 μM, preferably 0.1-2.5 μM, more preferably 0.2-1 μM, even more preferably 0.25-0.8 μM, even more preferably 0.3-0.75 μM, even more preferably 0.35-0.7 μM, even more preferably 0.4-0.6 μM, even more preferably 0.45-0.55 μM, and most preferably about 0.5 μM.
46. The method of any of embodiments 38-45, wherein the serum-free additive in step (i) is 0.1-10% (v/v) N2 additive, preferably 0.3-7.5% (v/v) N2 additive, more preferably 0.5-5% (v/v) N2 additive, more preferably 0.75%-2% (v/v) N2 additive, more preferably 0.9%-1.2% (v/v) N2 additive, and most preferably about 1% (v/v) N2 additive.
47. The method of any of embodiments 38-46, wherein the basal medium in step (i), step (ii), step (iii) and/or step (iv) is selected from DMEM, DMEM/F12, RPMI, IMDM, alphaMEM, Medium 199, Hams F-10, Hams F-12, wherein the basal medium is preferably DMEM, in particular wherein the basal medium is supplemented with pyruvate and/or non-essential amino acids, and/or comprises 1 g/l glucose.
48. The method of any one of embodiments 38-47, wherein in step (ii), the cultivation is carried out in the presence of (a) a gamma-secretase/NOTCH inhibitor, (b) FGF2, and (c) the serum-free additive for 36 to 60 hours, preferably for 42 to 54 hours, and most preferably for about 48 hours; and/or the cultivation is carried out in the presence of (a) a gamma-secretase/NOTCH inhibitor, (b) FGF2, (c) the serum-free additive, and (d) HGF for 36 to 60 hours, preferably for 42 to 54 hours, and most preferably for about 48 hours; and/or the cultivation is carried out in the presence of (a) a gamma-secretase/NOTCH inhibitor, (b) HGF, (c) the serum-free additive, and (d) knockout serum replacement (KSR) for 72 to 120 hours, preferably for 76 to 114 hours, more preferably for 84 to 108 hours, even more preferably for 90 to 102 hours, and most preferably for about 96 hours.
49. The method of any one of embodiments 38-48, wherein in step (ii), the gamma-secretase/NOTCH inhibitor is selected from the group DAPT, RO4929097, semagacestat (LY450139), avagacestat (BMS-708163), dibenzazepine (YO-01027), LY411575, IMR-1, L685458, wherein the gamma-secretase/NOTCH inhibitor is preferably DAPT.
50. The method of any one of embodiments 38-49, wherein in step (ii), the effective amount of FGF2 is 15-30 ng/ml, preferably 17.5-25 ng/ml, more preferably 18-22 ng/ml, even more preferably 19-21 ng/ml, and most preferably about 20 ng/ml; and/or
   the effective amount of HGF is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml; and/or
   the gamma-secretase/NOTCH inhibitor is DAPT, and the effective amount is 1-20 μM, preferably 2-19 μM, more preferably 3-18 μM, even more preferably 4-17 μM, even more preferably 5-16 μM, even more preferably 6-15 μM, even more preferably 7-14 μM, even more preferably 7.5-13 μM, even more preferably 8-12 μM, even more preferably 9-11 μM, and most preferably about 10 μM;
   the KSR is used in an amount of 6-14% (v/v), preferably 7-13% (v/v), more preferably 8%-12% (v/v), more preferably 9%-11% (v/v), and most preferably about 10% (v/v) KSR; in particular wherein the KSR is used in the presence of a reducing agent such as beta-mercaptoethanol and/or alpha-thioglycerol.
51. The method of any one of embodiments 38-50, wherein in step (iii), the effective amount of HGF is 1-15 ng/ml, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml;
   the KSR is used in an amount of 5-20% (v/v), preferably 6-17.5% (v/v), more preferably 7-15% (v/v), more preferably 8%-12% (v/v), more preferably 9%-11% (v/v), and most preferably about 10% (v/v) KSR; in particular wherein the KSR is used in the presence of a reducing agent such as beta-mercaptoethanol and/or alpha-thioglycerol.
52. The method of any of embodiments 38-51, wherein step (iii) is carried out for 7-11 days, preferably 8-10 days, and most preferably for about 9 days.
53. The method of any of embodiments 38-52, wherein in step (iv) the additional serum-free additive has a final concentration of 0.5-50 mg/ml albumin, 1-100 μg/ml transferrin, 0.1-10 μg/ml ethanolamine, 17.4-1744 nM selenium or a bioavailable salt thereof, in particular sodium selenite, 0.4-40 μg/ml L-carnitine, 0.05-5 μl/ml fatty acid additive, 0.0001-0.1 μg/ml triiodo-L-thyronine (T3) provided in the medium.
54. The method of any of embodiments 38-53, wherein the additional serum-free additive in step (iv) is 0.1-10% (v/v) B27, preferably 0.5-8% (v/v), more preferably 1-6% (v/v), even more preferably 1.5-4% (v/v), and most preferably about 2% (v/v) B27.
55. The method of any one of embodiments 38-54, wherein step (iv) is carried out for at least 30 days, preferably at least 35 days, more preferably at least 40 days, and even more preferably at least to 50 days.
56. The method of any one of embodiments 38-55, comprising, prior to step (i), a seeding step in which the pluripotent stem cells are seeded in a stem cell medium in the presence of a ROCK inhibitor, preferably wherein the seeding step is carried out 18-30 hours prior to step (i).
57. The method of embodiment 56, wherein the ROCK inhibitor is selected from the group consisting of Y27632, H-1152P, thiazovivin, fasudil, hydroxyfasudil, GSK429286A and RKI1447, preferably the ROCK inhibitor is selected from the group consisting of Y27632, H-1152P, thiazovivin, fasudil and hydroxyfasudil, more preferably the ROCK inhibitor is selected from the group consisting of Y27632 and H-1152P, wherein particularly preferably the ROCK inhibitor is Y27632.
58. The method of embodiment 56 or 57, wherein the ROCK inhibitor is Y27632 and is used at a concentration of 0.5-10 μM, preferably 1-9 μM, more preferably 2-8 μM, more preferably 3-7 μM, more preferably 4-6 μM, and most preferably at a concentration of about 5 μM; and/or wherein the stem cell medium is iPS-Brew XF.
59. The method of embodiment 58, wherein the stem cell medium comprises 5-20% (v/v), preferably 6-17.5% (v/v), more preferably 7-15% (v/v), more preferably 8%-12% (v/v), more preferably 9%-11% (v/v), and most preferably about 10% (v/v) KSR; and/or
   wherein the stem cell medium comprises 1-15 ng/ml FGF2, preferably 2.5-14 ng/ml, more preferably 5-13 ng/ml, even more preferably 7.5-12.5 ng/ml, even more preferably 8-12 ng/ml, even more preferably 9-11 ng/ml, and most preferably about 10 ng/ml FGF2.
60. An engineered skeletal muscle tissue, having multinuclear mature skeletal muscle fibers with satellite cells, and having no blood supply and/or no central nervous system control; in particular wherein the presence of skeletal muscle fibers is determined by staining of actinin and with DAPI.
61. The engineered skeletal muscle tissue of embodiment 60, wherein the skeletal muscle tissue is serum-free and/or does not comprise a differentiation- or maturation-related transgene, preferably wherein the skeletal muscle tissue does not comprise a myogenic transgene, more preferably wherein the skeletal muscle tissue does not comprise the transgene Pax7 or MyoD.
62. The engineered skeletal muscle tissue of embodiment 60 or embodiment 61, wherein the skeletal muscle tissue generates at least a contraction force of 0.3 millinewtons (mN) upon a stimulus of 100 Hz at 200 mA, preferably at least 0.4 mN, more preferably at least 0.5 mN, more preferably at least 0.6 mN, more preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN; more preferably at least 1.8 mN; more preferably at least 1.9 mN; and most preferably generates at least 2 mN.
63. The engineered skeletal muscle tissue of any one of embodiments 60-62, wherein the skeletal muscle tissue is formed by engineering, preferably wherein it has the engineered form of a ring, a ribbon, a strand, a patch, a pouch, or a cylinder, optionally wherein individual skeletal muscle tissues are fused, particularly wherein the skeletal muscle tissue has the form of a ring.
64. Mesodermally differentiated skeletal myoblast progenitor cells obtained according to step (i) according to embodiment 1 or embodiment 38, prepared by a method according to embodiment 1 (i) or according to embodiment 38 (i), characterized by the expression of the genes MSGN1 and/or TBX6, wherein the expression of MSGN1 and/or TBX6 can be determined by flow cytometry and/or immunostaining; and/or the mRNA SP5 is expressed, wherein the expression of SP5 can be determined by RNA sequencing.
65. Myogenically specified skeletal myoblast precursor cells obtained according to step (ii) according to embodiment 1 or embodiment 38, produced by a method according to embodiment 1 (i) to (ii) or according to embodiment 38 (i) to (ii), characterized by the expression of the gene PAX3, wherein the expression of PAX3 can be determined by flow cytometry and/or immunostaining; and/or the mRNA SIM1 is expressed, wherein the expression of SIM1 can be determined by RNA sequencing.
66. Skeletal myoblast cells obtained according to step (iii) according to embodiment 1 or embodiment 38, produced by a method according to embodiment 1 (i) to (iii) or according to embodiment 38 (i) to (iii), characterized by the expression of actinin, preferably wherein the expression of actinin can be determined by flow cytometry and/or immunostainings.
67. A satellite cell, obtained according to step (iii) according to embodiment 1 or embodiment 38, produced by a method according to embodiment 1 (i) to (iii) or according to embodiment 38 (i) to (iii), characterized by the expression of the gene Pax7, wherein the expression of Pax7 can be determined by flow cytometry and/or immunostainings, more preferably wherein satellite cells further express Ki67.
68. A mixture of skeletal myoblast cells according to embodiment 66 and satellite cells according to embodiment 67, wherein a proportion of satellite cells of the amount of all available cells is obtained of at least 10%, preferably at least 15%, more preferably at least 20%, even more preferably at least 30%, determined by expression of Pax7 by flow cytometry; and/or wherein a proportion of skeletal myoblasts of the amount of all cells present of at least 40% is obtained, preferably at least 50%, more preferably at least 60%, most preferably at least 70%, determined by expression of actinin by flow cytometry.
69. Skeletal myotubes obtained according to step (iv) of embodiment 1 or embodiment 38, prepared by a method according to embodiment 1 (i) to (iv) or according to embodiment 38 (i) to (iv), characterized by an anisotropic orientation of the actinin protein-containing sarcomere structure.
70. Use of a skeletal muscle tissue according to embodiments 60-63, and/or cells according to any of embodiments 64-68, and/or skeletal myotubes according to embodiment 69 in an in vitro drug assay; in particular wherein the drug assay is a toxicity assay, or an assay for skeletal muscle tissue function under the influence of pharmacological and gene therapeutic drug candidates.
71. A skeletal muscle tissue according to embodiments 60-63 and/or cells according to any of embodiments 64-68, and/or of skeletal myotubes according to embodiment 69 for use in medicine.
72. Satellite cells according to embodiment 67 for use in the therapy of damaged skeletal muscle and/or in the treatment of skeletal muscle diseases, preferably genetic skeletal muscle defects, in particular Duchenne muscular dystrophy and/or Becker-Kiener muscular dystrophy, and/or lysosomal storage diseases, in particular Pompe disease, preferably wherein the skeletal muscle disease is Duchenne muscular dystrophy.
73. An in vitro method for testing the efficacy of a drug candidate on a skeletal muscle tissue, comprising the steps of
   (a) providing a skeletal muscle tissue according to any of embodiments 60-63,
   (b) optionally inflicting damage on the skeletal muscle tissue, and
   (c) contacting the skeletal muscle tissue from step (a) or (b) with a drug candidate;
   preferably wherein the method further comprises determining the contraction force and/or structure of the skeletal muscle tissue and/or metabolic function and/or molecular parameters and/or protein biochemical parameters before and/or after step (c).
74. An in vitro method for testing the toxicity of a substance on a skeletal muscle tissue, comprising the steps of
   (a) providing a skeletal muscle tissue according to any of embodiments 60-63,
   (b) contacting the skeletal muscle tissue from step (a) with a substance to be tested.
   preferably wherein the method further comprises determining contraction force and/or skeletal muscle tissue structure and/or metabolic function and/or molecular parameters and/or protein biochemical parameters before and/or after step (b).
75. An in vitro method for testing the effect of nutrients and dietary supplements on skeletal muscle tissue performance, comprising the steps of
   (a) providing a skeletal muscle tissue according to any of embodiments 60-63,
   (b) contacting the skeletal muscle tissue from step (a) with a nutrient or dietary supplement to be tested,
   preferably wherein the method further comprises determining contraction force and/or skeletal muscle tissue structure and/or metabolic function and/or molecular parameters and/or protein biochemical parameters before and/or after step (b).
76. An in vitro method for testing the efficacy of a drug candidate on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
   (a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to any of embodiments 64-69,
   (b) optionally inflicting damage on the cells from step (a), and
   (c) contacting the cells of step (a) or (b) with a drug candidate;
   preferably wherein the method further comprises determining the expression of actinin and/or Pax7 before and/or after step (c), wherein the expression can be determined by flow cytometry and/or immunostainings.
77. An in vitro method for testing the toxicity of a substance to mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
   (a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to any of embodiments 64-69,
   (b) contacting the cells of step (a) with a substance to be tested,
   preferably wherein the method further comprises determining the expression of actinin and/or Pax7 before and/or after step (b), wherein the expression can be determined by flow cytometry and/or immunostainings.
78. An in vitro method for testing the effect of nutrients and dietary supplements on mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells, comprising the steps of:
   (a) providing mesodermally differentiated skeletal myoblast progenitor cells, myogenically specified skeletal myoblast progenitor cells, satellite cells, skeletal myoblast cells, skeletal myotubes, or a mixture of skeletal myoblast cells and satellite cells according to any of embodiments 64-69,
   (b) contacting the cells of step (a) with a nutrient or dietary supplement to be tested,
   preferably wherein the method further comprises determining the expression of actinin and/or Pax7 before and/or after step (b), wherein the expression can be determined by flow cytometry and/or immunostainings.
79. The method of any one of embodiments 1-59, wherein the skeletal muscle tissue generates at least a contraction force of 0.6 millinewtons (mN) upon a stimulus of 100 Hz, preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, and most preferably generates at least 2 mN.
80. The method of any of embodiments 1-59 or 79, wherein the skeletal muscle tissue generates at least a contraction force of 2 millinewtons (mN) upon a stimulus of 100 Hz, preferably at least 2.3 mN, more preferably at least 2.6 mN, even more preferably at least 3 mM, even more preferably at least 3.3 mN, even more preferably at least 3.6 mN, most preferably at least 4 mN.
81. The method of any of embodiments 1-59, 79 or 80, wherein the skeletal muscle tissue has a contraction speed of at least 3 mN/sec upon a stimulation of 100 Hz, preferably at least 4 mN/sec, more preferably at least 5 mN/sec, more preferably at least 6 mN/sec, even more preferably at least 6.5 mN/sec, even more preferably at least 7 mN/sec.
82. The method of any of embodiments 1-59 or 79-81, wherein the skeletal muscle tissue has a relaxation speed of at least 0.5 mN/sec upon termination of a stimulation of 100 Hz, preferably at least 0.7 mN/sec, more preferably at least 0.9 mN/sec more preferably at least 1 mN/sec, even more preferably at least 1.2 mN/sec, even more preferably at least 1.5 mN/sec.
83. The method of any one of embodiments 1-59 or 79-82, wherein the basal medium in step (iv) comprises an effective amount of creatine and/or triiodo-L-thyronine (T3).
84. The method of embodiment 83, wherein the effective amount of creatine in the basal medium increases the contraction force of the engineered skeletal muscle compared to a maturing in step (iv) without an effective amount of creatine.
85. The method of embodiment 83 or 84, wherein the effective amount of T3 in the basal medium shortens the contraction speed and/or relaxation speed of the engineered skeletal muscle compared to maturing in step (iv) without an effective amount of T3.
86. The method of any of embodiments 1-59, or 79-85, wherein the basal medium in step (iv) provides a final concentration of 0.1-10 mM creatine, preferably 0.2-6 mM creatine, more preferably 0.4-4 mM creatine, even more preferably 0.6-3 mM creatine, even more preferred 0.7-2.5 mM creatine, even more preferred 0.8-2 mM creatine, even more preferred 0.85-1.5 mM creatine, even more preferred 0.9-1.2 mM creatine, and most preferred about 1 mM creatine.
87. The method of any of embodiments 1-59, or 79-86, wherein the basal medium in step (iv) provides a final concentration of 0.001-1 μM triiodo-L-thyronine (T3), preferably 0.005-0.7 μM T3, more preferably 0.01-0.35 μM T3, even more preferred 0.04-0.0.2 μM T3, even more preferred 0.05-0.18 μM T3, even more preferred 0.06-0.15 μM T3, even more preferred 0.08-0.12 μM T3, even more preferred about 0.1 μM T3.
88. The method of any one of embodiments 1-59, or 79-87, wherein the skeletal muscle tissue has the property of being self-regenerating.
89. The method of embodiment 88, wherein the regenerative property is characterized by a recovered contractility and/or muscle recovery, preferably wherein said recovered contractility and/or muscle recovery is measured after irreversible muscle damage with cardiotoxin, more preferably wherein said recovered contractility and/or muscle recovery is measured 10-30 days after incubation with cardiotoxin.
90. The method of any of embodiments 1-59 or 79-89, wherein step (iv) is carried out for at least 50 days, more preferably at least 60 days, even more preferably at least 70 days, even more preferably at least 80 days.
91. An engineered skeletal muscle tissue produced by a method according to any one of embodiments 1-59 or 79-90.
92. The engineered skeletal muscle tissue of any one of embodiments 60-63 or 91, wherein the skeletal muscle tissue generates at least a contraction force of 0.6 millinewtons (mN) at a stimulus of 100 Hz, preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, more preferably at least 2 mN, more preferably at least 2.3 mN, more preferably at least 2.6 mN, even more preferably at least 3 mM, even more preferably at least 3.3 mN, even more preferably at least 3.6 mN, and most preferably at least 4 mN.
93. The engineered skeletal muscle tissue of any of embodiments 60-63 or 91-92, wherein the skeletal muscle tissue has a contraction speed of at least 3 mN/sec upon a stimulation of 100 Hz, preferably at least 4 mN/sec, more preferably at least 5 mN/sec, more preferably at least 6 mN/sec, even more preferably at least 6.5 mN/sec, even more preferably at least 7 mN/sec.
94. The engineered skeletal muscle tissue of any one of embodiments 60-63 or 91-93, wherein the skeletal muscle tissue has a relaxation speed of at least 0.5 mN/sec upon termination of a stimulation of 100 Hz, preferably at least 0.7 mN/sec, more preferably at least 0.9 mN/sec, more preferably at least 1 mN/sec, even more preferably at least 1.2 mN/sec, even more preferably at least 1.5 mN/sec.
95. Use of a skeletal muscle tissue according to embodiments 60-63 or 91-94, and/or cells according to any of embodiments 64-68, and/or skeletal myotubes according to embodiment 69 in an in vitro drug assay; in particular wherein the drug assay is a toxicity assay, or an assay for skeletal muscle tissue function under the influence of pharmacological and gene therapeutic drug candidates.
96. A skeletal muscle tissue according to embodiments 60-63 and 91-94, and/or cells according to any of embodiments 64-68, and/or of skeletal myotubes according to embodiment 69 for use in medicine.

### DESCRIPTION OF THE FIGURES

FIG. 1. Schematic of the differentiation protocol in 2D cell culture. Differentiation protocol for the directed differentiation of human pluripotent stem cells (hPSCs) into skeletal myoblasts and satellite cells in 2D. The hPSCs are seeded the day before. For the mesodermal induction, cells are cultured from day 0 to day 4 in mesodermal induction medium (DMEM with 1 g/l glucose, supplemented with pyruvate) with CHIR-99021, LDN193189, and FGF-2. For myogenic specification, cells are cultured from day 4 to day 6 in medium containing DAPT and FGF-2, followed by cultivation from day 6 to day 8 in medium containing DAPT, FGF-2, and HGF, followed by cultivation from day 8 to day 12 in a medium containing DAPT, HGF, and “knockout serum replacement” (KSR). For the myogenic expansion and maturation, cells are cultured from day 12 to day 21 in medium containing HGF and KSR. For the myogenic maturation, cells are cultured from day 21 in medium containing albumin, transferrin, ethanolamine, selenium, carnitine, fatty acids, and T3. In addition, the medium comprises serum-free additive N-2 from day 0 to day 21.

FIG. 2. Schematic of cell differentiation from pluripotent stem cells into myotubes and satellite cells. Schematic representation of the differentiation of pluripotent stem cells into myotubes, comprising the following stages: (i) pluripotent stem cells, (ii) presomitic mesodermal cells, (iii) myoblasts with satellite cells, (iv) myotubes with satellite cells, and (v) myotubes with satellite cell niche. In addition, the expression sequence of marker genes during the different stages of differentiation is shown. Expression of Oct4 is characteristic of pluripotent stem cells. The expression of MSGN1 and Tbx6 is characteristic of presomitic mesodermal cells. Pax3 is mainly expressed during the transition from presomitic mesodermal cells to myoblasts. Pax7 expression is characteristic of the presence of satellite cells and is first expressed at the end of the presomitic mesodermal stage and at the beginning of the myoblast stage. While Pax7 expression is highest at the myoblast stage, Pax7 expression flattens out by the myotube stage, but remains as a sign of satellite cell niche formation. The expression of MyoD is present the strongest in myoblasts and is also detectable in myotubes. The expression of myogenin and actinin is characteristic of myotubes and is hardly expressed in myoblasts. Myotubes form satellite cell niches, the muscle stem cell niche. Satellite cell niches are Pax7 positive and quiescent. The cell cycle is activated upon muscle damage, at which time the cells are also Ki67 positive.

FIG. 3. Fluorescence microscopy of skeletal muscle cells and satellite cells. Immunostaining of myogenic cells in representative cell culture after 21 days (Example 1). The fluorescence images show the expression of skeletal muscle-specific transcription factors PAX7 (top row left), MyoD (middle row left), and myogenin (bottom row left). PAX7 detects satellite cells; MyoD and myogenin detect skeletal myoblasts and/or skeletal myotubes. Furthermore, the fluorescence images show cell nuclei (nuclei, right column) and the expression of actin (middle column). Scale: 100 μm.

FIG. 4. Gene expression patterns during the differentiation of human pluripotent stem cells (hPSCs) into skeletal muscle cells analyzed by RNA sequencing. The directed differentiation shows gene expression patterns similar to embryonic skeletal muscle development. Expression values (“reads per kilobase million, RPKM”) of genes typical of pluripotency and paraxial mesoderm are shown graphically over the time of differentiation and maturation (FIGS. 4A and 4B). In RNA sequencing, genes typical of pluripotency, such as NANOG, POU5F1, and ZFP42, show high expression on days 0 and 1. NANOG and POU5F1 show the highest expression at day 0; ZFP42 shows the highest expression on day 1. Genes typical of the paraxial mesoderm, such as MSGN1, TBX6, and MEOX1, show a high expression on days 1-8. MSGN1 shows the highest expression on day 1; TBX6 shows the highest expression on day 4; and MEOX shows the highest expression on day 8. Expression values (“reads per kilobase million, RPKM”) of typical genes for skeletal muscle-specific transcription factors and sarcomeres over the time of differentiation and maturation are graphically shown (FIGS. 4C and 4D). Skeletal muscle-specific transcription factors, such as PAX3, PAX7, and MYOD1, show the highest expression at days 8, 29, and 60, respectively. Genes typical of sarcomeres, such as ACTN2, DMD, and MYH3, show the highest expression on day 60.

FIG. 5. Analysis of the efficiency in directed differentiation of human pluripotent stem cells (hPSC) into skeletal muscle cells. Flow cytometric determination of the proportion of muscle cells (actinin as well as myogenin or MyoD-positive) and satellite cells (PAX7-positive) from four independent pluripotent stem cell lines (iPSC (WT 1), iPSC (WT 2), DMD iPSC, corrected DMD iPSC). The proportion of actinin-positive cells is from 71 to 77.6% in the four cell lines; the proportion of myogenin-positive cells is from 41.4% to 60.4% in the four cell lines; the proportion of MyoD-positive cells is from 40% to 54.1% in the four cell lines; the proportion of PAX7-positive cells is from 33.4% to 43.8% in the four cell lines.

FIG. 6. Production of engineered skeletal muscle tissue (ESM) from skeletal myoblasts derived from hPSCs. (A) Schematic of the ESM cultivation protocol. The 21-day-old cell pool from Example 1 is poured into an extracellular matrix (collagen/Matrigel) and cultured in ring-shaped moulds (left panel) for 7 days under expansion conditions. The formed rings are subsequently transferred to stretching apparatuses (center image) and further cultured under maturation conditions. After another 4 weeks, ESM function is measured in organ baths (right image); scale: 5 mm. (B) Representative contraction force curves of the engineered skeletal muscle tissue at different stimulation frequencies: 1 Hz (dashed line: eight single contractions having a single duration of approximately 500 ms with a force of contraction (“FOC”) of approximately 0.5 millinewtons each), 10 Hz (solid line: incipient tetanus with a force of contraction (“FOC”) of approximately 1 millinewton, individual contractions graphically distinguishable), 100 Hz (dash-dot line: fully developed tetanus with a force of contraction (“FOC”) of approximately 2.2 millinewtons). (C) Force of contraction (FOC) in millinewtons (mN) of the skeletal muscle tissue dependent on electrical stimulation frequency; n=3. For a stimulus of 1 Hz, the contraction force averages 0.5 millinewtons; for a stimulus of 10 Hz, the contraction force averages 0.9 millinewtons; for a stimulus of 20 Hz, the contraction force averages 1.1 millinewtons; for a stimulus of 40 Hz, the contraction force averages 1.4 millinewtons; for a stimulus of 60 Hz, the contraction force averages 1.55 millinewtons; for a stimulus of 80 Hz, the contraction force averages 1.6 millinewtons; for a stimulus of 100 Hz, the contraction force averages 2.1 millinewtons.

FIG. 7. Production of bioengineered skeletal muscle (BSM) from hPSC. (A) Schematic of the BSM cultivation protocol. Human induced pluripotent stem cells are cast into ring-shaped moulds in a collagen/Matrigel hydrogel and differentiated into skeletal muscle tissue in 3D. The formed rings are transferred to stretching apparatuses at day 21 and further cultured under maturation conditions. After an additional 4 weeks, BSM function is typically measured in organ baths. Specifically, for this purpose, human induced pluripotent stem cells are first dispersed in a collagen/Matrigel hydrogel and conditioned for 24 h in Brew XF containing Y-27632 and KSR (day −1). The cells are then cultured from day 0 to day 4 in medium containing CHIR-99021, LDN193189 and FGF-2. Cells are cultured from day 4 to day 6 in medium containing DAPT and FGF-2, followed by a cultivation from day 6 to day 8 in medium containing DAPT, FGF-2 and HGF, followed by a cultivation from day 8 to day 12 in medium containing DAPT, HGF and “knockout serum replacement” (KSR). The cells are cultured from day 12 to day 21 in medium containing HGF and KSR. From day 21 to day 50, cells are cultured in maturation medium on static stretching apparatuses, i.e., under mechanical stretching. In addition, from day 0 to day 50, the medium comprises the serum-free additive N-2. (B) Representative contraction force curves of the engineered skeletal muscle tissue at different stimulus frequencies: 1 Hz (dashed line: eight single contractions with a single duration of approximately 600 ms, each with a force of contraction (“FOC”) of approximately 0.7 millinewtons) and 100 Hz (solid line: fully developed tetanus with a force of contraction (“FOC”) of approximately 1.1 millinewtons). (C) Force of contraction (FOC) in millinewtons (mN) of skeletal muscle tissue dependent on the electrical stimulation frequency; n=3. For a stimulus of 1 Hz, the contraction force averages 0.3 millinewtons; for a stimulus of 10 Hz, the contraction force averages 0.5 millinewtons; for a stimulus of 20 Hz, the contraction force averages 0.55 millinewtons; for a stimulus of 40 Hz, the contraction force averages 0.6 millinewtons; for a stimulus of 60 Hz, the contraction force averages 0.65 millinewtons; for a stimulus of 80 Hz, the contraction force averages 0.72 millinewtons; for a stimulus of 100 Hz, the contraction force averages 0.9 millinewtons.

FIG. 8. Fluorescence microscopy of skeletal muscle tissue produced by ESM and BSM methods. Immunostaining of actin and DNA in representative skeletal muscle tissues prepared by the ESM (Examples 1 and 2) and BSM (Example 3) methods. Fluorescence images show multinuclear mature skeletal muscle fibers, characterized by the characteristic stripe pattern (stained actin). Scale: 50 μm (ESM) and 10 μm (BSM).

FIG. 9. Enhancement of ESM function by creatine treatment. A) Experimental procedure of ESM maturation over 5 or 9 weeks with additional administration of 1 mM creatine over 4 weeks. B) Force of contraction (FOC) in response to 100 Hz electric field stimulation in ESM after 5 and 9 weeks in culture; culture was as shown in A with (right bars) or without (left bars) addition of creatine; n=3 per group; *p<0.05 by Student's t test.

FIG. 10. Enhancement of ESM function by thyroid hormone treatment. A) Experimental scheme of ESM maturation over 5 or 9 weeks with additional administration of 0.1 μmol/L triiodo-L-thyronine (T3) over 4 weeks. (B) Maximum speed of tension (+dFOC/dt) and relaxation (−dFOC/dt) at and after 100 Hz electric field stimulation with representative curves. Comparison of ESM treated with (gray) and without (black) T3 in week 5 (day 56) and week 9 (day 84); n=4-11 per group; *p<0.05 by Student's t test. C) Protein content of myosin heavy chain protein 2 (MYH2; fast isoform), myosin heavy chain protein 7 (MYH7; slow isoform), and myosin heavy chain protein 3 (MYH3; embryonic isoform) in 9 week ESM cultures with (gray bars) and without T3 (black bars); n=3 per group; *p<0.05 by Student's t-test.

FIG. 11: Regenerative capacity of engineered skeletal muscle. A) RNA detection by RNA sequencing (in reads per kilobase million, RPKM) of skeletal muscle progenitor cell/stem cell markers in 2D culture on culture day 22 and 60, as well as in ESM on culture day 60 (ESM were prepared from 2D cultures on day 22). *p<0.05 by 1-way ANOVA and Tukey's multiple comparison test. B) Immunofluorescence staining of skeletal muscle cell progenitor cells (Pax7) in ESM: Pax7 (bright nuclei), laminin, f-actin (elongated muscle cell bodies), and nucleus in ESM (left) and 2D (right) cultures on culture day 60; bars: 20 μm. Magnifications represent satellite cell niches (skeletal muscle cell precursors) in ESM and 2D culture. C) Experimental scheme for cardiotoxin (CTX) injury. ESM were incubated with 25 μg/ml CTX for 24 hr. The irradiated group was treated with 30 Gy (gamma irradiation) 24 hours before CTX injury to inhibit the cell proliferation and regeneration associated therewith. D) Force of contraction (FOC) at 100 Hz tetanus of the ESM without (left bars) or with gamma irradiation (right bars) at the indicated time points after CTX injury (25 μg/ml) or vehicle treatment (Veh.); n=3-4, *p<0.05 vs control day+2, *p<0.05 by 1-way ANOVA and Tukey's multiple comparison test. E) Immunostaining of sarcomeric actinin and nuclei in ESM 21 days after CTX injury in the groups with and without gamma irradiation according to the scheme in A—muscle growth in the non-radiated control group is due to proliferation and differentiation of new muscle cells from muscle cell precursors in ESM. Bar: 50 μm

### EXAMPLES

The following examples are intended to further illustrate, but not limit, the invention. The examples describe technical features, and the invention also relates to combinations of the technical features presented in this section. Methods and materials that were used in all examples are described after the examples.

### Example 1: Directed Differentiation of Human Pluripotent Stem Cells (hPSCs) into Skeletal Muscle Cells and Satellite Cells in 2D Cell Culture

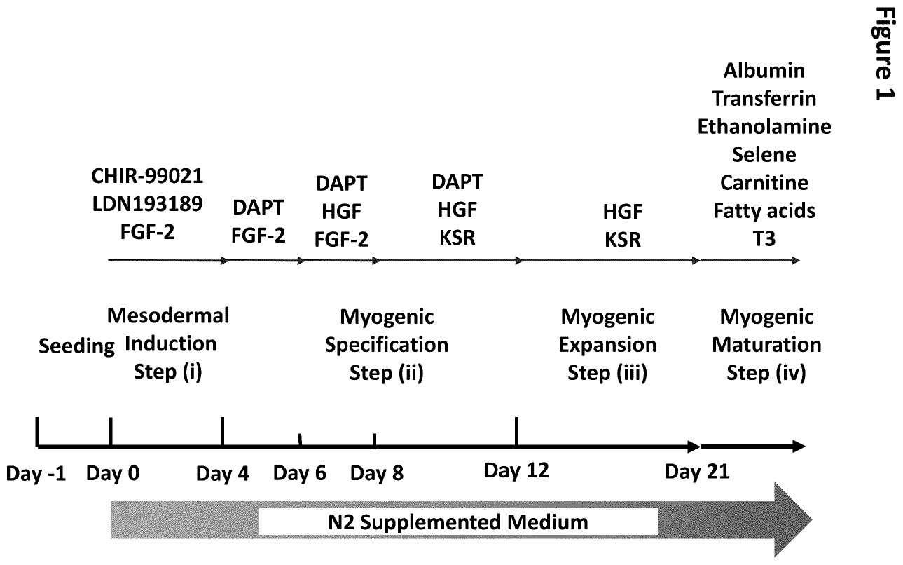

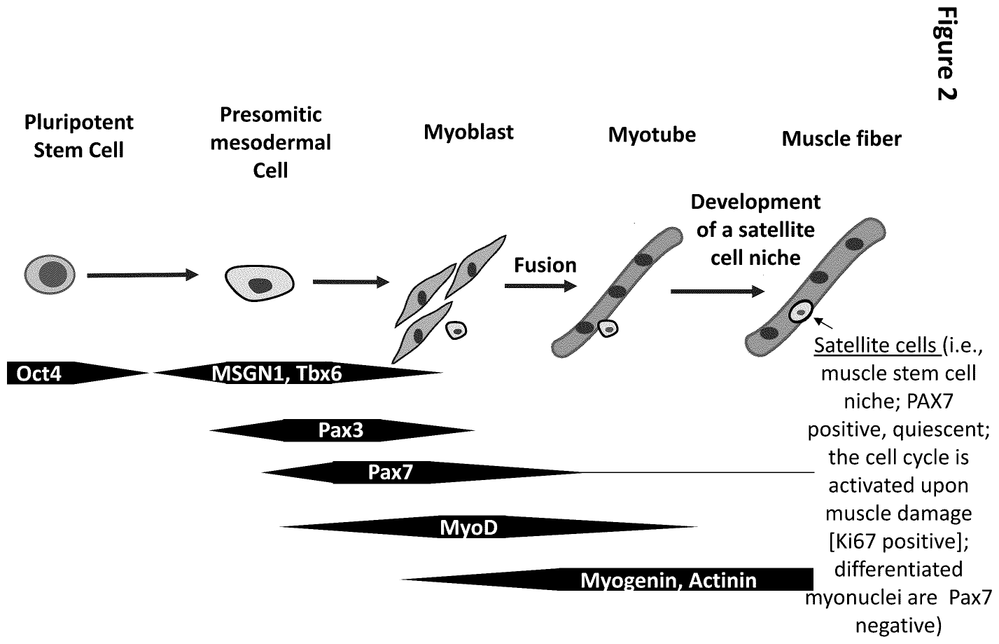

A method was developed for the directed differentiation of induced pluripotent stem cells into skeletal muscle cells and satellite cells in 2D cell culture. The method described here is transgene- and serum-free. Human skeletal myoblasts, skeletal myotubes, and satellite cells can be generated in high purity by this method. In this method, a specific temporal sequence of agents (small molecules and inhibitors and stimulators) was used to induce the differentiation of human pluripotent stem cells. Different genes were expressed at different differentiation stages of pluripotent stem cells. The typical gene expression during differentiation is also called gene expression patterns. These gene expression patterns are also undergone during embryonic skeletal muscle development in the human body. The schematic of the differentiation protocol is shown in FIG. 1, and it shows the sequence of the different agents added to the medium. In addition, FIG. 1 shows the differentiation stages undergone during differentiation into skeletal myoblasts/myotubes and satellite cells, i.e., the induction of mesoderm differentiation, the induction of myogenic specification, the (myogenic) expansion and maturation into skeletal myoblasts and satellite cells, and the maturation into skeletal myotubes and satellite cells.

To perform the method, human pluripotent stem cells were plated out at a density of 1.7×10⁴ cells/cm 2 on Matrigel-coated plates the previous day and cultured in the presence of 12 ml of StemMACS™ iPS-Brew XF medium with 5 μM Rock Inhibitor (Stemolecule Y27632) (the method for coating cell culture plates with Matrigel at the end of Example 1), so that the cell culture was approximately 30% confluent on the following day (day 0). However, the optimum cell number for plating must be determined individually for each cell line.

Culturing in N2-FCL medium induced mesoderm differentiation of the pluripotent stem cells. On each of days 0, 1, 2, and 3, the medium was replaced with 15 ml of N2-FCL medium and changed daily. N2-FCL medium: DMEM containing 1 g/l glucose and L-alanyl-L-glutamine (GlutaMAX™) supplemented with pyruvate (Gibco), 1% Pen/Strep (Invitrogen), 1% serum-free additive N-2 (100×) (Thermo Scientific), 1% non-essential amino acids (100×) (MEM-NEAA, Invitrogen), 10 ng/ml recombinant bFGF (Peprotech), 10 μM CHIR-99021 (Stemgent), 0.5 μM LDN193189 (Stemgent)).

Myogenic specification was induced by culturing in N2-FD, N2-FHD and N2-HKD media. On days 4 and 5, the medium was replaced with N2-FD medium and changed daily. N2-FD medium: DMEM with 1 g/l glucose and L-alanyl-L-glutamine (GlutaMAX™) supplemented with pyruvate (Gibco), 1% Pen/Strep (Invitrogen), 1% serum-free additive N-2 (100×) (Thermo Scientific), 1% non-essential amino acids (100×) (MEM-NEAA, Invitrogen), 20 ng/ml recombinant bFGF (Peprotech), 10 uM DAPT (TOCRIS).

On days 6 and 7, the medium was replaced with N2-FHD medium and changed daily. N2-FHD medium: DMEM with 1 g/l glucose and L-alanyl-L-glutamine (GlutaMAX™) supplemented with pyruvate (Gibco), 1% Pen/Strep (Invitrogen), 1% serum-free additive N-2 (100×) (Thermo Scientific), 1% non-essential amino acids (100×) (MEM-NEAA, Invitrogen), 20 ng/ml recombinant bFGF (Peprotech), 10 μM DAPT (TOCRIS), 10 ng/ml recombinant HGF (Peprotech).

On days 8, 9, 10 and 11, the medium was replaced by N2-HKD medium and changed daily. N2-HKD medium: DMEM with 1 g/l glucose and L-alanyl-L-glutamine (GlutaMAX™) supplemented with pyruvate (Gibco), 1% Pen/Strep (Invitrogen), 1% serum-free additive N-2 (100×) (Thermo Scientific), 1% non-essential amino acids (100×) (MEM-NEAA, Invitrogen), 0.1 mM 2-mercaptoethanol (Invitrogen), 10 μM DAPT (TOCRIS), 10 ng/ml recombinant HGF (Peprotech), 10% knockout serum replacement (Life Technologies).

By culturing in N2-HK medium, the cells were myogenically expanded and matured into skeletal myoblasts and satellite cells. On days 12 to 20, the medium was replaced with N2-HK medium (expansion medium) and changed every other day. N2-HK medium: DMEM with 1 g/l glucose and L-alanyl-L-glutamine (GlutaMAX™) supplemented with pyruvate (Gibco), 1% Pen/Strep (Invitrogen), 1% serum-free additive N-2 (100×) (Thermo Scientific), 1% non-essential amino acids (100×) (MEM-NEAA, Invitrogen), 0.1 mM 2-mercaptoethanol (Invitrogen), 10 ng/ml recombinant HGF (Peprotech), 10% knockout serum replacement (Life Technologies).

From day 21, cells were either further cultured on cell culture plates, frozen, or used in the method of Example 2. When cells were further cultured, the medium was replaced with differentiation medium (maturation medium). Maturation medium: DMEM with 1 g/l glucose and L-alanyl-L-glutamine (GlutaMAX™) supplemented with pyruvate (Gibco), 1% Pen/Strep (Invitrogen), 1% serum-free additive N-2 (Thermo Scientific), 1% B27 serum-free additive (Invitrogen). Skeletal myoblasts, skeletal myotubes and satellite cells are generated by further culturing on cell culture plates.

To track the directed differentiation during the described cultivation steps, the gene expression patterns of the cells were determined over a 60-day period using RNA sequencing. RNA sequencing was used to determine the increase and decrease in the expression of specific genes, i.e., the entry and exit at specific differentiation or maturation stages was analyzed.

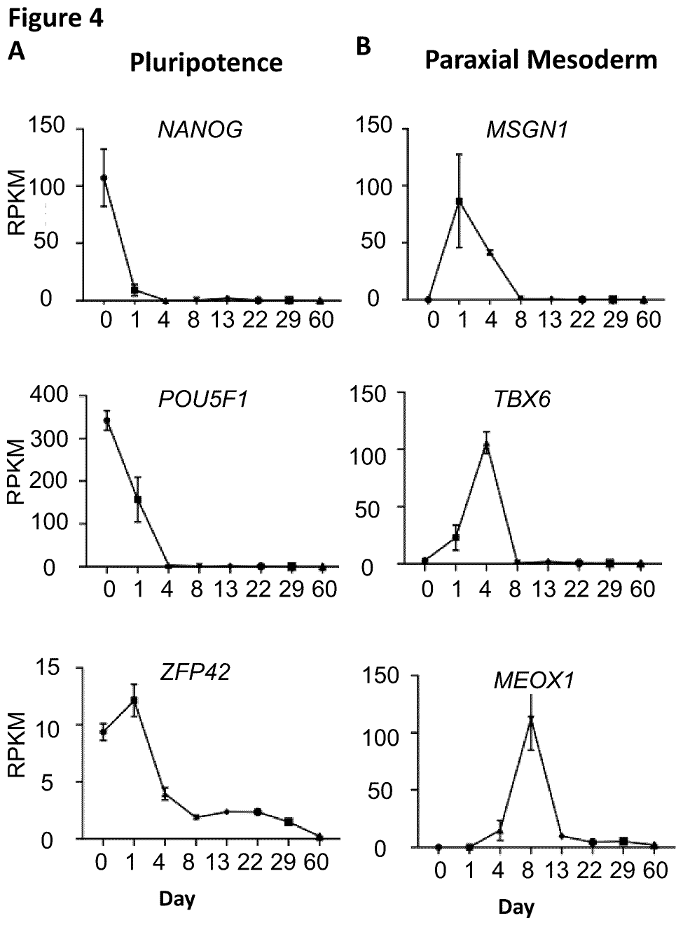

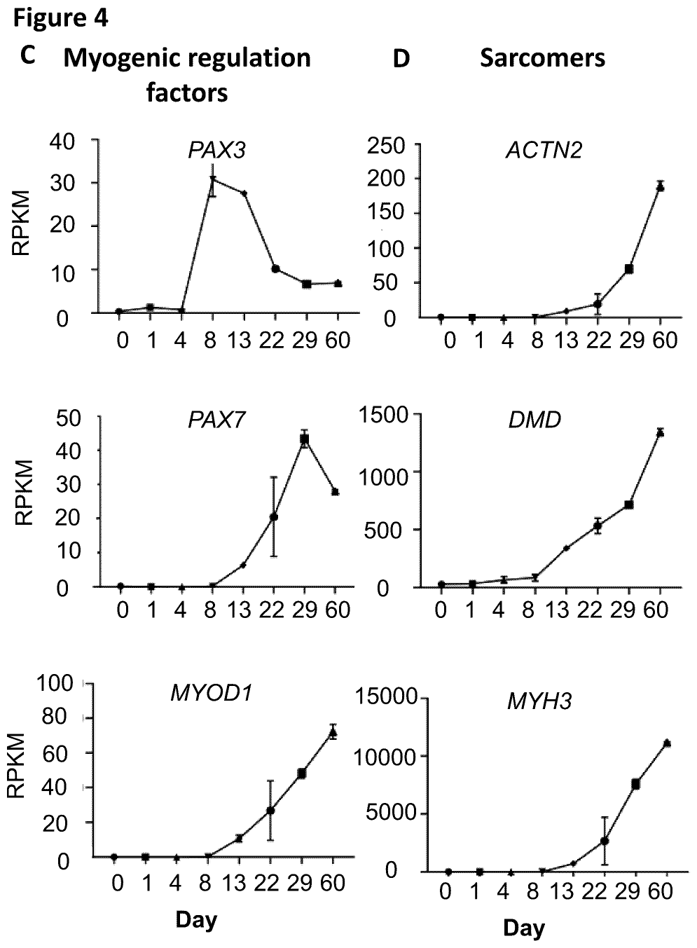

Specifically, the expression of specific genes for pluripotency, paraxial mesoderm, skeletal muscle-specific transcription factors, and sarcomeres were measured. Genes typical of pluripotency, such as NANOG, POU5F1, and ZFP42, showed high expression at days 0 and 1 (the day after the seed step and the following day) (FIG. 4a). NANOG and POU5F1 show the highest expression on day 0; ZFP42 shows the highest expression on day 1 (FIG. 4a). Genes typical of the paraxial mesoderm, such as MSGN1, TBX6, and MEOX1, show high expression on days 1-8 (FIG. 4b). MSGN1 shows the highest expression at day 1; TBX6 shows the highest expression at day 4; and MEOX shows the highest expression at day 8 (FIG. 4b). Skeletal muscle-specific transcription factors, such as PAX3, PAX7, and MYOD1, show the highest expression at days 8, 29, and 60, respectively (FIG. 4c). Genes typical of sarcomeres, such as ACTN2, DMD, and MYH3, show the highest expression at day 60 (FIG. 4d). Moreover, the gene expression patterns show a steep increase or decrease of the different markers especially during the first 21 days. For example, TBX6 and MEOX1 are strongly expressed only on days 4 and 8, respectively, whereas expression is at least 4-fold weaker on the other days (FIG. 4d). This time course indicates a homogeneous progression of the differentiation process.

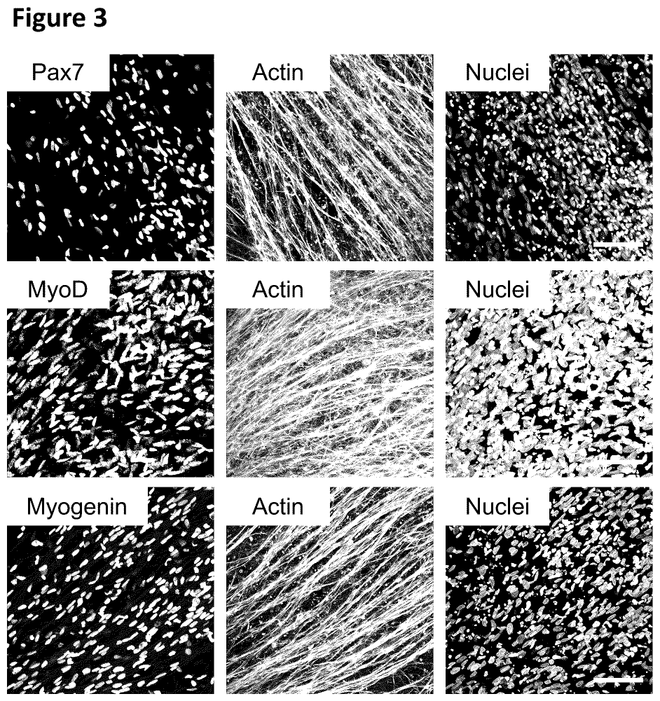

To determine differentiation using a second independent method, the inventors analyzed the cells after the 21-day differentiation method using fluorescence microscopy. This involved staining DNA of the cells with Hoechst, and immunostaining actin and skeletal muscle-specific transcription factors (Pax7, MyoD, and myogenin). After 21 days, fluorescence images showed a high percentage of cells expressing Pax7, MyoD, and myogenin (FIG. 3). Thus, using another method, it was shown that a cell population of myogenic cells was generated by the differentiation protocol.

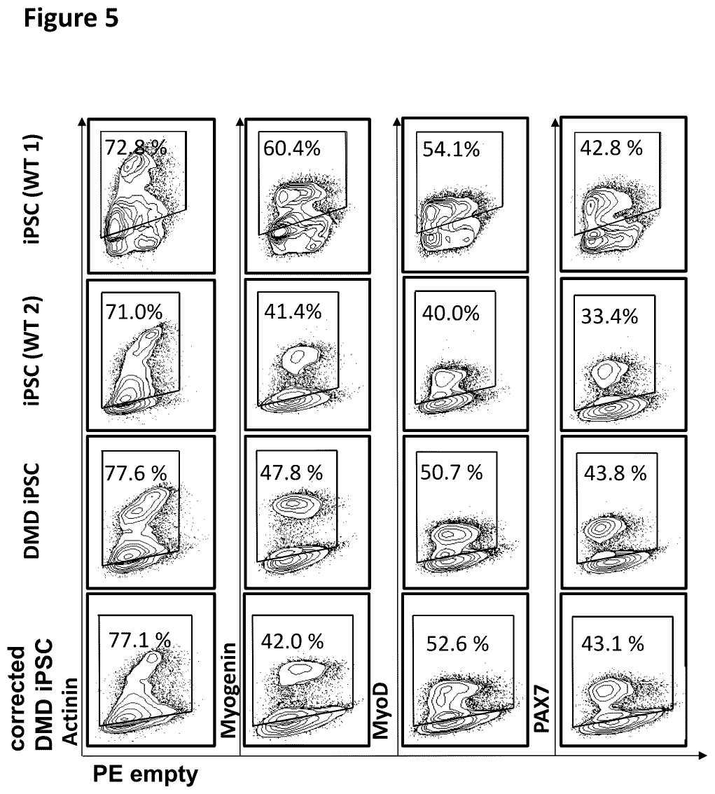

To determine differentiation by a third independent method, the cells were analyzed by flow cytometry. Flow cytometry, as used here, measures the expression of skeletal muscle-specific factors using immunostaining. Specifically, the proportion of skeletal myoblasts and skeletal myotubes (expression of the markers actinin, myogenin, MyoD) and satellite cells (expression of the marker PAX7) was determined in four independent pluripotent stem cell lines (iPSC (WT 1), iPSC (WT 2), DMD iPSC, corrected DMD iPSC) (FIG. 5). The percentage of actinin-positive cells was between 71 and 77.6% in the four cell lines; the percentage of myogenin-positive cells was between 41.4% and 60.4% in the four cell lines; the percentage of MyoD-positive cells was between 40% and 54.1% in the four cell lines; the percentage of PAX7-positive cells was between 33.4% and 43.8% in the four cell lines (FIG. 5).

Flow cytometry also showed that the analyzed cells produced skeletal myoblast- and skeletal myotube-specific, as well as satellite cell-specific markers in high purity (>70% actinin-positive and >30% PAX7-positive myocytes).

Thus, three different methods were used to measure that pluripotent stem cells were differentiated into a skeletal myoblast-containing cell pool and thus underwent mesodermal induction, myogenic specification, and myogenic maturation.

Materials and Methods

The following pluripotent stem cell lines were used: TC1133 (iPSC WT1; Baghbaderani et al. Stem Cell Reports 2015), iPSC WT2, DMD iPSC (DMD Del; Long et al. Sci Adv 2018), corrected DMD iPSC (Long et al. Sci Adv 2018). In the DMD iPSCs stem cell line, the X-linked dystrophin gene (DMD) is mutated, which is also mutated in Duchenne muscular dystrophy (DMD) disease and causes the disease. To prepare Matrigel-coated cell culture plates, BD Matrigel (Basement Membrane Matrix Growth Factor Reduced) was diluted in a ratio of 1:30 in ice-cold PBS and immediately stored at 4° C. To prepare Matrigel-coated plates, a 1:120 Matrigel dilution was made with ice-cold PBS. 0.1 ml/cm 2 of the dilution was added to the cell culture flasks. The flasks were stored at 4° C. at least overnight and for a maximum of 2 weeks. Before use, the plates were placed in the 37° C. incubator for at least half an hour.

For passaging (e.g., to detach cells for cryopreservation), cells were washed once with 3 ml TrypLE (Invitrogen), followed by incubation in 5 ml TrypLE for approximately 7 minutes at room temperature. The TrypLE was then washed out and the digestion was stopped with 10 ml of N2-HK medium containing 5 uM Rock inhibitor. In order to induce clumps, the cell suspension was pipetted using a 10-ml pipette. The separation of cells must be gentle enough not to reduce cell viability. Cells were counted using a CASY counter (by adding 20 μl of the cell suspension to 10 ml of CASY buffer). Cells were pelleted at 100×g for 10 min at room temperature. The supernatant was removed and the pellet was gently resuspended in N2-HK medium containing 5 uM Rock inhibitor. Cells were plated out on Matrigel-coated plates at a density of 60-70 000 cells/cm 2 in N2-HK medium containing 5 μM Rock Inhibitor. Starting the next day, N2-HK medium was replaced every other day for 9 days.

For cell freezing (e.g., on day 21, cryopreservation), cells were washed once with 3 ml TrypLE (Invitrogen) and then incubated in 5 ml TrypLE for approximately 7 minutes at room temperature. Afterwards, the TrypLE was washed out, and the digestion was stopped with 10 ml of N2-HK medium containing 5 μM Rock inhibitor. In order to induce clumps, the cell suspension was pipetted using a 10-ml pipette. The separation of cells must be gentle enough not to reduce cell viability. Cells were counted using a CASY counter (by adding 20 μl of the cell suspension to 10 ml of CASY buffer). Cells were pelleted at 100×g for 10 min at room temperature. The supernatant was removed and the pellet was gently resuspended in N2-HK medium containing 5 μM Rock inhibitor and 10% DMSO (Sigma) at 4° C. 10×10 6 cells were frozen in 2 ml per cryovial using Mr Frosty (Thermo) overnight at −80° C. Cells were then transferred to −150° C.

For the RNA extraction, cell lysates embedded in Trizol reagent (Thermo Fisher) were homogenized by vortexing. For every 1 ml of Trizol reagent, 200 μl of chloroform was added (AppliChem). Reagent tubes were tightly closed and inverted five times followed by 5 min incubation at room temperature. Samples were then centrifuged at 10,000-12,000×g for 15 minutes. The aqueous phase containing RNA was transferred to fresh reagent tubes, followed by the addition of 500 μl of isopropanol (Roth) to precipitate the RNA. The reagent tubes were vortexed, allowed to stand at room temperature for 10 min, and then centrifuged at 12,000×g for an additional 10 min. The supernatant was removed and 1 ml of 70% EtOH/diethyl pyrocarbonate (DEPC) H₂O was added to wash the pellet. After gently tapping the reagent tube to dissolve and wash the pellet, the samples were centrifuged one more time at 12,000×g for 5 minutes, and the supernatant was removed. The pellets were left open for 5-10 minutes until the remaining liquid had evaporated, and the RNA was resuspended in DEPC H₂O. RNA concentration and quality were determined using a Nanodrop ND-1000. Prior to sequencing, quality and RNA integrity were further analyzed using the Fragment Analyzer from Advanced Analytical (Standard Sensitivity RNA Analysis Kit (DNF-471)). RNA-Seq libraries were generated using a modified strand-specific massively parallel cDNA sequencing (RNA-Seq) protocol (Illumina: TruSeq Stranded Total RNA (Cat. No. RS-122-2301)). The protocol was optimized to keep the rRNA content in the dataset below 5% (RiboMinus™ technology). The remaining whole transcriptome RiboMinus™ RNA is suitable for direct sequencing. The ligation step was optimized to increase ligation efficiency (>94%), and PCR protocols were adjusted for an optimum final product of the library. For accurate quantification of cDNA libraries, a fluorometric-based system, the quantiFluor™ dsDNA system from Promega was used. The size of the final cDNA libraries was determined using the dsDNA 905 Reagent Kit (Fragment Analyzer from Advanced Bioanalytical), with an average size of 300 bp.

The libraries were pooled (merged) and sequenced on an Illumina HiSeq 4000 (Illumina), generating 50 bp single-end reads (30-40×10\hat{ }6 reads/sample). Sequence images were converted to BCL files using the Illumina software BaseCaller, which were demultiplexed to fastq files using bcl2fastq v2.17.1.14. Quality was evaluated using FastQC version 0.11.5 (Andrews, 2014). Sequence reads were mapped to the human genome reference library (UCSC version hg19 with Bowtie 2.0 (Langmead and Salzberg, 2012)). Then, the number of mapped reads for each identified gene was counted, and DESeq2 software was used to assess differential gene expression (Anders and Huber, 2010). Reads per kilobase transcript per million (RPKM) were calculated based on the Ensembl transcript length extracted from biomaRt (v2.24).

For flow cytometry, single cell suspensions were prepared by digesting cells with TrypLE Select (Thermo Fisher). Cells were resuspended in culture medium, centrifuged at 300 g for 5 minutes, and fixed in 4% formalin (Histofix, Roth). After fixation, cells were centrifuged again and resuspended in block buffer (PBS containing 1 mg/ml BSA (Sigma-Aldrich), 5% FCS (Thermo Fisher), and 0.1% Triton 100× (Sigma)). After 10 min of blocking, cells were pelleted by centrifugation and resuspended in blocking buffer with primary antibodies (sarcomeric α-actinin 1:4,000 (Sigma-Aldrich); Pax7 1:50 (DSHB); MyoD 1:100 (DAKO); myogenin 1:50 (DSHB)) or appropriate IgG1 isotype control for 45 min at 4° C.

Cells were washed twice with PBS, followed by a wash step in blocking buffer and subsequent incubation in secondary antibody (1:1000 anti-mouse 488 [A-11001] or 633 [A-21052], Thermo Fisher) and Hoechst (10 ng/ml; Thermo Fisher) for 30 min at 4° C. Cells were washed with PBS and finally resuspended in PBS for analysis. 10,000 live cell events were analyzed per sample. Measurements were carried out on an LSRII SORP cytometer and analyzed using DIVA software (BD Biosciences).

### Example 2: Production of Engineered Skeletal Muscle (ESM) Tissue from Skeletal Myoblasts and Satellite Cells Derived from Pluripotent Stem Cells (Cells from Example 1

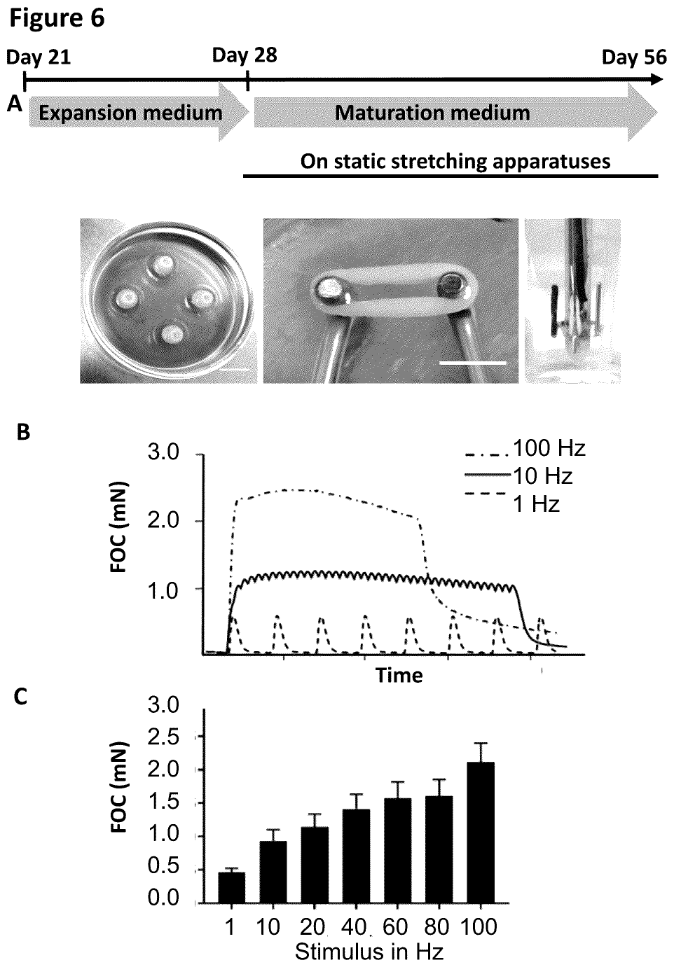

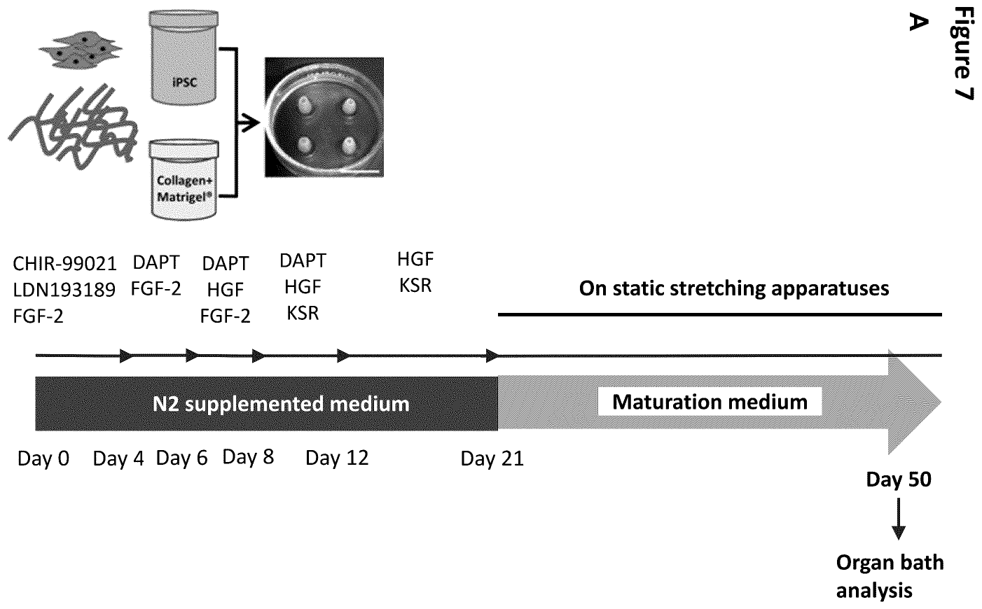

For the construction of engineered skeletal muscle tissue, the cells obtained in Example 1 (cells from day 21) were used as starting material and mixed with an extracellular matrix. By the mixing with an extracellular matrix, the cells are dispersed into a matrix to generate a three-dimensional skeletal muscle tissue. This method is also serum-free and transgene-free. Thus, the reproducibility for producing skeletal muscle tissue is increased, because all the required substances and their concentration have been defined. By this method, a force-generating skeletal muscle tissue can be generated that contracts in a controlled manner in response to an electrical stimulus. A specific temporal sequence of agents and physical stimuli is used, which are shown schematically in FIG. 6A and described in detail below.

To build the engineered skeletal muscle tissue, the cells from Example 1 (cells from 20 day 21) were mixed with an extracellular matrix and cast into ring moulds to support the self-assembly of the cells into a contractile skeletal muscle. This means that the cells were either (a) dissociated from a differentiated cell culture according to Example 1, or (b) frozen cells from Example 1 were used. (See below for a detailed description of how to thaw cells).

To mix the cells from Example 1 with the extracellular matrix, a master mix was mixed in a 50-ml reaction tube on ice. A 2-ml pipette was used to add the collagen. The following exact pipetting sequence was followed:

| ESM number (250 μl per ESM) | | | |
|---|---|---|---|
| All volumes are given in μl | 4 | 8 | 16 |
| Bovine collagen (6.5 mg/ml) | 144 | 288 | 575 |
| 2xDMEM serum-free | 144 | 288 | 575 |
| NaOH 0.1N | 27 | 55 | 109 |
| Matrigel | 100 | 200 | 400 |
| Cell suspension volume | 630 | 1260 | 2545 |
| Cell number (×10⁶) | 5 | 10 | 20 |
| Total volume | 1050 | 2100 | 4200 |

Alternatively, the master mix was pipetted according to the following volumes:

| ESM number (180 μl per ESM) | | | |
|---|---|---|---|
| All volumes are given in μl | 1 | 8 | 48 |
| Bovine collagen (6.84 mg/ml) | 35 | 280 | 1680 |
| 2xDMEM serum-free | 35 | 280 | 1680 |
| NaOH 0.1N | 5 | 40 | 240 |
| Matrigel | 19 | 152 | 912 |
| Cell suspension volume | 100 | 800 | 4800 |
| Cell number (×10⁶) | 1 | 8 | 48 |
| Total volume | 194 | 1552 | 9312 |

The master mix was poured into the ring moulds, and the ring moulds were carefully transferred into an incubator to allow the mixture to rest at 37° C. for 1 hour. After the incubation period, 8 ml of expansion medium containing 5 μM Rock inhibitor per mould was carefully added (FIG. 6A, left panel). Expansion medium (N2-HK medium): DMEM with 1 g/l glucose and L-alanyl-L-glutamine (GlutaMAX™) supplemented with pyruvate (Gibco), 1% Pen/Strep (Invitrogen), 1% serum-free additive N-2 (Thermo Scientific), 1% non-essential amino acids (MEM-NEAA, Invitrogen), 0.1 mM 2-mercaptoethanol (Invitrogen), 10 ng/ml recombinant HGF (Peprotech), 10% knockout serum replacement (Life Technologies).

Cells were thus cultured in expansion medium for 7 days. On days 1, 3, and 5, the medium was replaced with fresh expansion medium (N2-HK medium; without Rock inhibitor). After casting, the mixture compressed in the ring mould, so that the mixture was fully compressed after 24 hours.

After 7 days, the moulded rings were transferred to expansion trays in 6-well plates (FIG. 6A, middle panel). Thus, the cells were further cultured under a physical stimulus, i.e., mechanical stretching. In addition, the maturation of the cells was induced by maturation medium by adding 5 ml of maturation medium per well.

Maturation medium: DMEM containing 1 g/l glucose and L-alanyl-L-glutamine (GlutaMAX™) supplemented with pyruvate (Gibco), 1% Pen/Strep (Invitrogen), 1% N serum-free additive N-2 (Thermo Scientific), 2% B27 serum-free additive (Invitrogen).

To mature the cells into skeletal myotubes and satellite cells, the maturation medium was changed every other day of the subsequent 6 weeks of maturation.

To experimentally test the production of engineered skeletal muscle tissue, the generated skeletal muscle tissue was analyzed using fluorescence microscopy. The characteristic stripe pattern proves that multinuclear skeletal muscle fibers have formed to produce force-generating skeletal muscle.

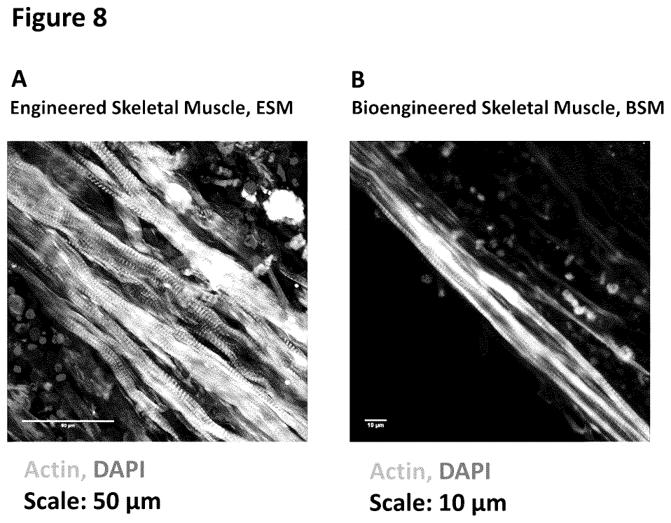

The inventors visualized the structural protein actin of the eukaryotic cytoskeleton using immunostaining, and the DNA in the nuclei was stained with the dye DAPI. The fluorescence images show the characteristic stripe pattern, demonstrating that multinucleated mature skeletal muscle fibers were formed by the method (FIG. 8A). Immunostaining demonstrated that the engineered skeletal muscle tissue exhibited the architecture of mature multinuclear muscle fibers.

Furthermore, to also test the artificially generated muscle tissue functionally, the inventors performed contraction experiments (FIG. 6A, right panel). These contraction experiments in organ baths measure the contraction frequency and contraction force of the produced skeletal muscle tissue in response to electrical stimulation.

For this purpose, the skeletal muscle tissue in the form of a ring was isometrically transferred in organ baths (Fohr Medical Instruments) containing Tyrode's solution (in mmol/L: 120 NaCl, 1 MgCl₂, 1.8 CaCl₂), 5.4 KCl, 22.6 NaHCO₃, 4.2 NaH₂PO₄, 5.6 glucose, and 0.56 ascorbate) at 37° C. and continuous gassing with 5% CO₂ and 95% O₂. The ESMs were mechanically stretched at 125 μm intervals until the maximum force amplitude (force of contraction=FOC) was observed. FOC measurements were performed at electric field stimulation frequencies in the range of 1-100 Hz (4 ms rectangular pulses; 200 mA).

The results of the contraction experiments are shown in FIGS. 6B and 6C. FIG. 6B shows representative contraction force curves of the engineered skeletal muscle tissue at different stimulation frequencies. At a stimulation of 1 Hz (dashed line), eight single contractions with a single duration of approximately 0.5 seconds were recorded; at a stimulation of 10 Hz (solid line), an incipient tetanus was measured; and at a stimulation of 100 Hz (dash-dot line), a fully developed tetanus was detected. FIG. 6C shows the contraction force of the engineered skeletal muscle tissue as a function of the stimulus frequency. The force of contraction (“FOC”) was measured in millinewtons (mN) of skeletal muscle tissue dependent on electrical stimulus frequency (n=3). At a stimulus of 1 Hz, the force of contraction averages 0.5 millinewtons; at a stimulus of 10 Hz, the force of contraction averages 0.9 millinewtons; at a stimulus of 20 Hz, the force of contraction averages 1.1 millinewtons; at a stimulus of 40 Hz, the force of contraction averages 1.4 millinewtons; at a stimulus of 60 Hz, the contraction force averages 1.55 millinewtons; at a stimulus of 80 Hz, the contraction force averages 1.6 millinewtons; at a stimulus of 100 Hz, the contraction force averages 2.1 millinewtons.

The skeletal muscle tissues tested showed a reproducible contraction frequency and contraction force in response to stimulation frequencies between 1 Hz and 100 Hz. At a single stimulation of 1 Hz, a contraction and complete relaxation took approximately 0.5 seconds. Because the contraction and relaxation time is approximately 0.5 seconds, a beginning or complete tetanus was recorded at higher stimulation frequencies. A tetanus is also formed in natural skeletal muscle tissues at an increased stimulation frequency, so that the engineered skeletal muscle tissue behaves analogously to natural skeletal muscle tissue in this regard. Furthermore, the inventors were able to show that the contraction force of the muscle tissue increases with increasing contraction frequency. These properties are consistent with native skeletal muscle tissue, which also exhibits single contractions and tetanic contractions, as well as a positive force-frequency relationship in response to electrical stimulation. In contrast to the engineered skeletal muscle tissue, in natural muscle tissues the electrical impulses are triggered by a neurotransmitter stimulus (acetylcholine) from the motor endplate.

Thus, the described method generated an engineered muscle tissue that shows a characteristic formation of multinuclear muscle fibers (myotubes) and that generates force in response to electrical stimulation.

Materials and Methods

For the dissociation of cells from a cell culture (volumes stated for a T75 cell culture flask), cells were washed once with 3 ml TrypLE (Invitrogen) and then incubated in 5 ml TrypLE for approximately 7 minutes at room temperature. The TrypLE was washed out, and the digestion was stopped with 10 ml expansion medium containing 5 μM Rock inhibitor. In order to induce clumps, the cell suspension was triturated using a 10-ml pipette. The separation of cells must be gentle enough to not reduce cell viability. Cells were counted using a CASY counter (by adding 20 μl of the cell suspension to 10 ml of CASY buffer). Cells were pelleted at 100×g for 10 min at room temperature. The supernatant was removed, and the pellet was gently resuspended in the appropriate volume of expansion medium containing 5 μM Rock inhibitor, depending on the number of ESMs (see master mix). The cell suspension was placed on ice.

For thawing cells, a vial was removed from the −152° C. freezer. Cells were quickly thawed in a water bath at 37° C. for 2 minutes. The vial was sprayed with alcohol and transferred under the cell culture hood. The contents of the cryovial were transferred to a 15 ml reaction tube using a 2 ml serological pipette. The cryovial was washed with 1 ml of expansion medium at room temperature with 5 μM Rock inhibitor, and the expansion medium was added dropwise to the cells to avoid osmotic shock. Another 8 ml of expansion medium containing 5 μM Rock inhibitor were added slowly. The suspension was pipetted up and down no more than twice before cell counting to avoid cell damage. Cells were counted using a CASY counter (by adding 20 μl of the cell suspension to 10 ml of CASY buffer). Cells were pelleted at 100×g for 10 min at room temperature. The supernatant was removed, and the pellet was gently resuspended in the appropriate volume of expansion medium containing 5 μM Rock inhibitor; depending on the number of ESMs, a defined volume of cell suspension was prepared (see master mix). The cell suspension was placed on ice.

### Example 3: Production of Engineered Skeletal Muscle Tissue from Pluripotent Stem Cells (Bioengineered Skeletal Muscle, BSM

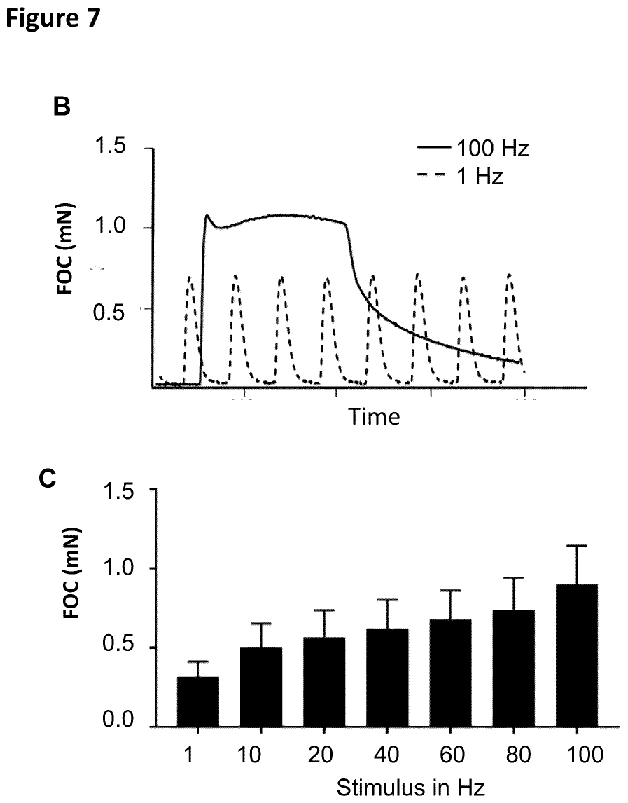

In this example, pluripotent stem cells and an extracellular matrix are used to build engineered skeletal muscle tissue (BSM). In contrast to Examples 1 and 2, no transition from Matrigel-coated cell culture plates to an extracellular matrix occurred in the production of BSM. Instead, human induced pluripotent stem cells were dispersed/embedded directly into a defined extracellular matrix. Self-assembly of pluripotent stem cells into skeletal muscle tissue was supported in the extracellular matrix in the presence of chemical and physical stimuli. This method is also serum-free and transgene-free, so all required substances and their concentrations were defined. Thus, the differentiation and maturation of human pluripotent stem cells into skeletal myotubes and satellite cells (skeletal muscle fibers) was controlled. The schematic of the differentiation protocol is shown in FIG. 7A and illustrates the sequence of the different agents added to the medium and the physical stimulation on stretching apparatuses. During the method depicted in FIG. 7A, mesoderm differentiation was induced (day 0-4), myogenic specification was induced (day 4-12), the cells matured into skeletal myoblasts and satellite cells (day 12-21), and finally matured into skeletal myotubes and satellite cells (day 21-50).

To perform the method, induced pluripotent stem cells were dissociated from a cell culture the day before, counted, and the pellet was gently resuspended in an appropriate volume of medium (iPS-Brew XF with 5 uM Rock Inhibitor, 10% KO serum replacement (Life Technologies) with 10 ng/ml bFGF (Peptrotech)). Stem cells were placed on ice as a cell suspension.

To mix the human pluripotent stem cells with collagen/Matrigel and pour into ring moulds, the master mix was mixed in a 50-ml reaction tube on ice. A 2-ml pipette was used to add the collagen and the following exact pipetting sequence was followed.

| BSM number (180 μl pro BSM) | |
|---|---|
| All volumes are given in μl | 4 |
| Bovine collagen (6.5 mg/ml) | 125 |
| 2xDMEM serum-free | 125 |
| NaOH 0.1N | 24 |
| Matrigel | 96 |
| Cell suspension volume | 448 |
| Cell number (×10⁶) | 3.2 |
| Total volume | 818 |

The master mix was poured into the ring moulds. The ring moulds were carefully transferred to an incubator to allow the mixture to rest at 37° C. for 1 hour. After the incubation period, 8 ml of medium per mould (iPS-Brew XF containing 5 μM Rock Inhibitor, 10% KO serum replacement (Life Technologies) containing 10 ng/ml bFGF (Peptrotech)) were carefully added.

Culturing in N2-FCL medium induced mesoderm differentiation of the pluripotent stem cells. Twenty-four hours after casting, the medium was replaced with N2-FCL medium. On days 1, 2, and 3, the medium was replaced daily with fresh N2-FCL medium. (See Example 1 for composition).

Myogenic specification was induced by culturing in N2-FD, N2-FHD, and N2-HKD media. On days 4 and 5, the medium was replaced with N2-FD medium and changed daily (see Example 1 for composition). On days 6 and 7, the medium was replaced by N2-FHD medium and changed daily. (See Example 1 for composition). On days 8, 9, 10, and 11, the medium was replaced with N2-HKD medium and changed daily (see Example 1 for composition).

On days 12 to 20, the medium was replaced by N2-HK medium (expansion medium) and changed every other day (see Example 1 for composition). By culturing in expansion medium, the cells were matured into skeletal myoblasts.

On day 21, the formed rings were transferred onto stretching apparatuses in 6-well plates and further cultured under maturation conditions. Thus, the cells were further cultured under a physical stimulus, i.e., mechanical stretching. In addition, the maturation of the cells was induced by maturation medium by adding 5 ml of maturation medium per well (see Example 2 for the composition of maturation medium). To mature the cells into skeletal myotubes and satellite cells, the maturation medium was changed every other day of the subsequent 4 weeks of maturation.

To experimentally test the production of engineered skeletal muscle tissue from induced pluripotent stem cells, the generated skeletal muscle tissue was analyzed using fluorescence microscopy, as in Example 2. As in Example 2, the structural protein actin of the eukaryotic cytoskeleton was visualized using immunostaining, and the DNA in the nuclei was stained with the dye DAPI. The fluorescence images showed the characteristic stripe pattern, as in Example 2, demonstrating the formation of multinuclear mature skeletal muscle fibers (FIG. 8b). Thus, the BSM also exhibits multinuclear mature skeletal muscle fibers formed by the method. Furthermore, to test the artificially generated muscle tissue functionally, the inventors performed contraction experiments, as in Example 2. These contraction experiments in organ baths measure the contraction frequency and contraction force of the produced skeletal muscle tissue in response to electrical stimulation.

The results of the contraction experiments are shown in FIGS. 7B and 7C. FIG. 7B shows representative contraction force curves of the engineered skeletal muscle tissue at different stimulus frequencies. At a stimulation of 1 Hz (dashed line), eight single contractions were recorded with a single duration of approximately 0.5 seconds; at a stimulation of 100 Hz (solid line), a fully developed tetanus was detected. FIG. 7C shows the contraction force of the engineered skeletal muscle tissue dependent on stimulus frequency. The force of contraction (“FOC”) was measured in millinewtons (mN) of skeletal muscle tissue (n=3). At a stimulus of 1 Hz, the force of contraction averaged 0.3 millinewtons; at a stimulus of 10 Hz, the force of contraction averaged 0.5 millinewtons; at a stimulus of 20 Hz, the force of contraction averaged 0.55 millinewtons; at a stimulus of 40 Hz, the force of contraction averaged 0.6 millinewtons; at a stimulus of 60 Hz, the contraction force averaged 0.65 millinewtons; at a stimulus of 80 Hz, the contraction force averaged 0.72 millinewtons; at a stimulus of 100 Hz, the contraction force averaged 0.9 millinewtons.

These contraction experiments demonstrate that the BSM also generates force in response to electrical stimulation. The skeletal muscle tissues tested showed reproducible contraction frequency and contraction force in response to stimulation frequencies between 1 Hz and 100 Hz, and the contraction and relaxation times after a single stimulus were approximately 0.6 seconds. In addition, the ESM and BSM show the same characteristics in terms of tetanus formation and increase in contraction force. Like the ESM described in Example 2, the BSM develops a tetanus at an increased stimulation frequency, such as 100 Hz. Also like in Example 2, the contraction force of the BSM increases with increasing stimulus frequency.

Both of these properties are analogous to a contraction behavior in natural muscle tissue, since in natural skeletal muscle, tetanus is also formed and contraction force increases with increased frequency of stimulation. Similar to natural skeletal muscles, the engineered skeletal muscle tissues showed single contractions and tetanic contractions as well as a positive force-frequency relationship in response to electrical stimulation.

Thus, engineered skeletal muscle tissues of Examples 2 and 3 (ESM and BSM) behave analogously to natural skeletal muscle tissues in response to electrical stimulation.

### Example 4—Increasing the Function of the Engineered Skeletal Muscle Tissue

To further increase the function of engineered skeletal muscle, e.g. the contraction force can be increased by adding specific molecules. In this example, we specifically tested the enhancement of the contraction force, as well as contraction and relaxation times, in response to the addition of creatine and an increased concentration of thyroid hormone T3 (triiodo-L-thyronine (T3); from 3 to 100 nmol/L in the maturation medium in step iv). Here, the procedure according to Examples 1 and 2 was performed first. In contrast to Example 2, the maturation medium was supplemented with creatine or an increased concentration of T3 either between days 28 and 56 or between days 56 and 84 of the method.

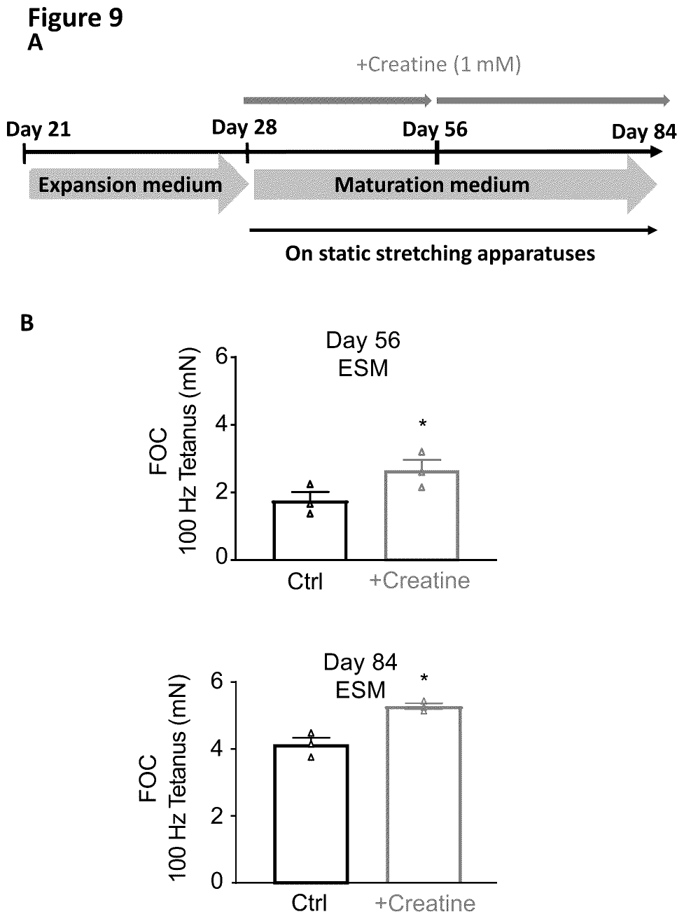

Creatine supplementation: when the maturation medium was supplemented with 1 mM creatine from day 28 of the procedure to day 56, the force of contraction (FOC) increased from 1.8 mN to 2.5 mN during a tetanic contraction at 100 Hz stimulation (FIG. 9B, top). Thus, this medium addition increased the force of contraction by 39%. Furthermore, a possible increase in contraction force in a prolonged method was tested. For this purpose, the method was extended for additional 4 weeks as described in Example 2, and the medium was supplemented with 1 mM creatine during this time. With a supplementation of the maturation medium with 1 mM creatine to from day 56 of the procedure to day 84, the force of contraction (twitch tension) increased from 4.0 mN to 5.2 mN during a tetanic contraction at 100 Hz stimulation (FIG. 9B, bottom). This medium additive thus increased the force of contraction by 30%.

From this, it follows that the addition of creatine to the maturation medium significantly increased the contraction force in both experiments.

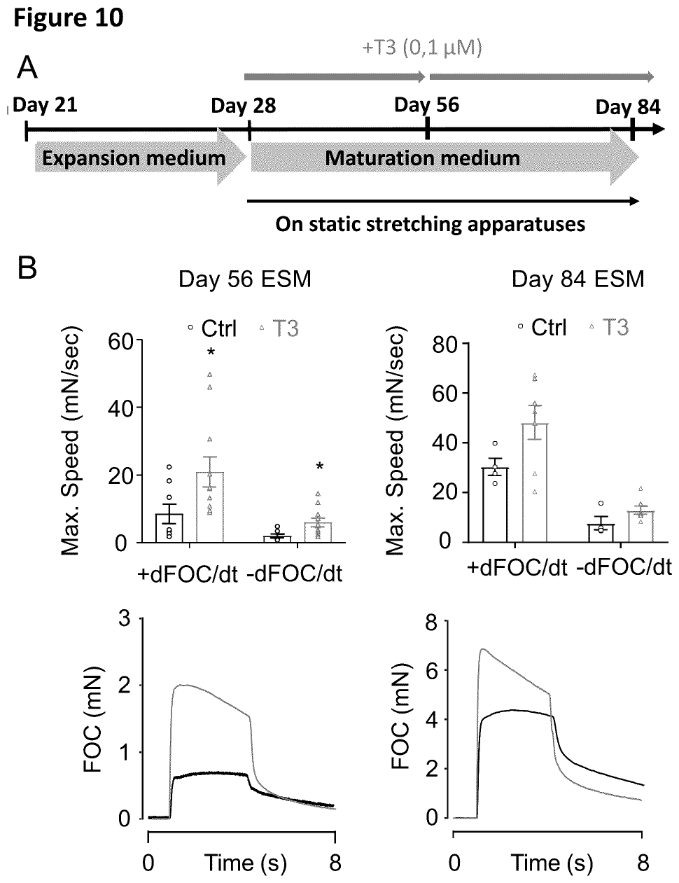

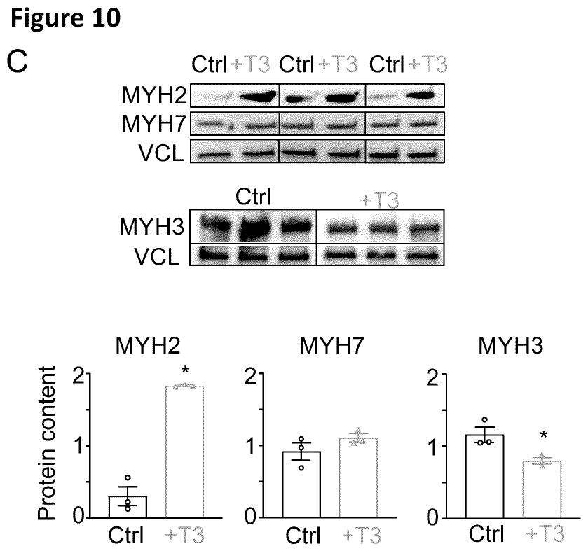

Supplementation with T3: With a supplementation of the maturation medium with 0.1 μM T3 from day 28 of the method to day 56, the contraction and relaxation speeds decreased significantly, as determined by a Student's T test (FIG. 10B). Furthermore, contraction and relaxation speeds decreased when the method was prolonged as described in Example 2, wherein the medium was supplemented with 0.1 μM T3 between days 56 and 84 (FIG. 10B). Thus, the engineered skeletal muscles respond faster to a tetanic stimulation and relax faster after termination of the stimulation.

It can be assumed that generally, a maturation medium with an increased T3 concentration leads to an improvement of skeletal muscle contractility in the sense of an acceleration of the contraction and relaxation times.

To investigate the molecular cause of this improved muscle function, the expression of different proteins was analyzed by Western blots. MYH2 is the heavy chain of the fast myosin (MYH2; fast myosin heavy chain); MYH7 is the heavy chain of the slow myosin (MYH7; slow myosin heavy chain); MYH3 is the heavy chain of the embryonic myosin (MYH3; embryonic myosin heavy chain). Protein expression was analyzed at day 84. As shown in FIG. 10C, the protein expression of MYH2 is significantly increased with a four-week addition of 0.1 μM T3. Based on three independent experiments, the expression is increased at least 5-fold. The expression of MYH7 remained unchanged by an addition of 0.1 μM T3. MYH3 expression was reduced by approximately half on average. These protein expression data support the functional data from FIGS. 10B, as the reduced response time of the engineered skeletal muscle can be explained by an increased expression of the fast myosin (MYH2) isoform by T3.

In conclusion, it was shown that an addition of creatine and/or T3 during maturation increased the function of the engineered skeletal muscle. Specifically, it was shown that an addition of creatine greatly increased the contraction force. In addition, it has been shown that an addition of T3 increases the reaction speed of the engineered skeletal muscle. This increase in function is supported by the increased expression of MYH2.

It can also be assumed that the increase in function of the engineered skeletal muscle will occur in the same manner when an engineered skeletal muscle tissue is prepared according to Example 3 (BSM), and creatine and/or T3 is then added to the maturation medium.

### Example 5—Regenerative Capacity of Engineered Skeletal Muscle Tissues

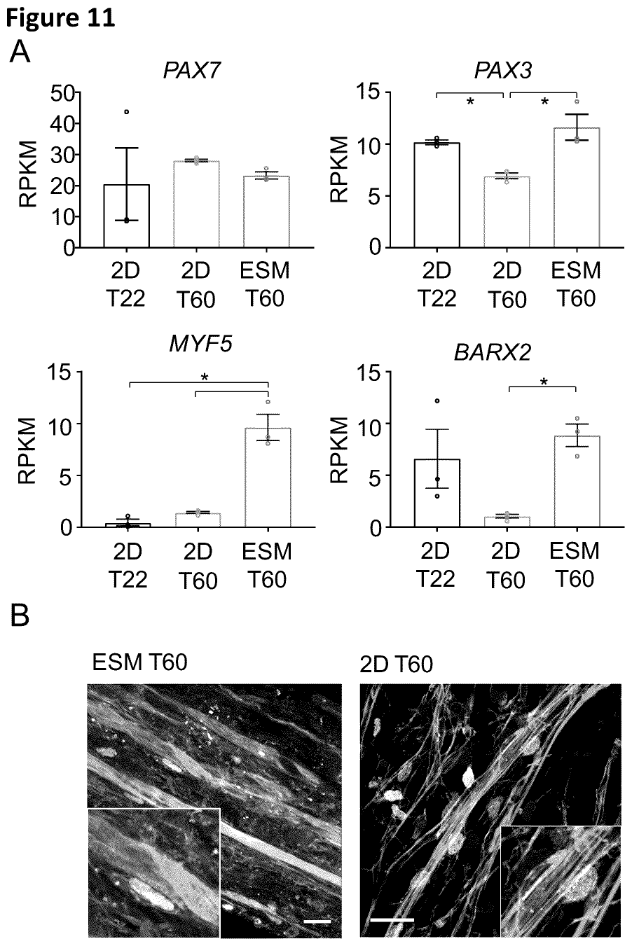

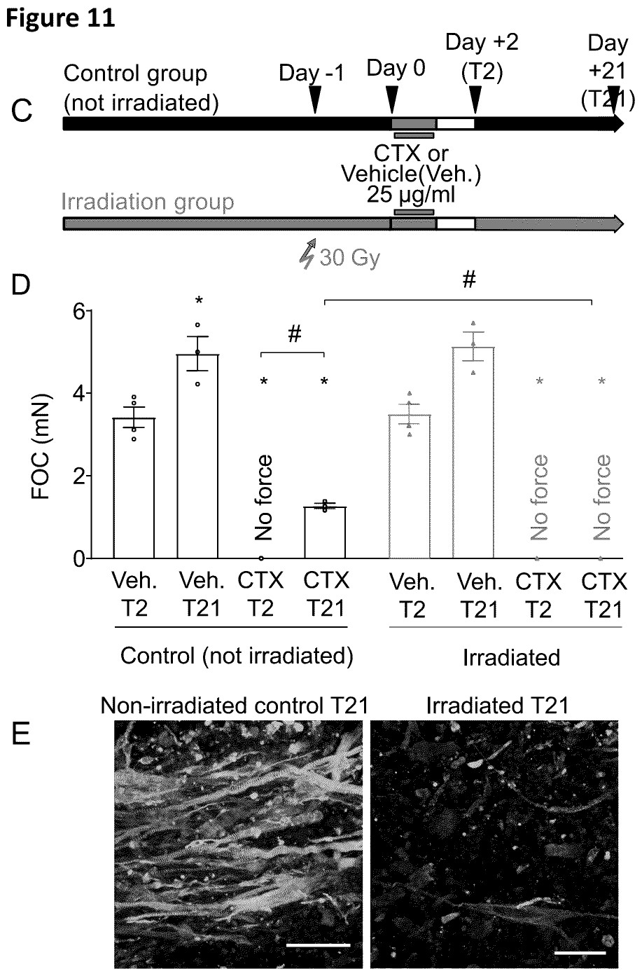

In order to be able to use engineered skeletal muscle tissue, for example, as an implant or as a model for testing regeneration or muscle growth-inducing drugs, the engineered skeletal muscle tissue ideally has a regenerative property. This regenerative property is characterized by the fact that injuries to the engineered skeletal muscle tissue can be repaired. For this repair process, an engineered skeletal muscle tissue requires cells with regenerative properties, for example, satellite cells (skeletal muscle progenitor cells). In FIG. 11A, protein expression of markers expressed in skeletal muscle cell precursors was analyzed (PAX7, PAX3, MYF5, and BARX2). In contrast to 2D cultures, all four markers were clearly expressed in ESM at culture day 60 of the method. Furthermore, PAX3, MYF5, and BARX2 were more highly expressed in engineered skeletal muscle than in skeletal muscle cells cultured in a 2D plate. This is an indication that in engineered skeletal muscle tissue, skeletal muscle cell precursors are maintained and additionally proliferate, in contrast to parallel 2D cultures. FIG. 11B then also shows well-differentiated satellite cell niches in ESM; sporadic and less differentiated satellite cell niches were also seen in 2D cultures analogous to the method described here.

To test the regenerative property, engineered skeletal muscle tissue (60 days old) was incubated with the muscle toxin cardiotoxin (25 μg/ml) for 24 hours. Contraction force was measured 2 and 21 days after incubation (FIG. 11C). As shown in FIG. 11D, engineered skeletal muscle tissues showed no contractions 2 days after incubation with CTX, and 21 days after incubation, engineered skeletal muscle contracted again with a contraction force of 1 mM. Thus, the engineered skeletal muscle is capable of regeneration. In comparison, a skeletal muscle additionally treated with gamma radiation (30 Gy) did not recover from incubation with CTX. This indicates that regeneration of engineered skeletal muscles is dependent on activation of the contained skeletal muscle cell precursor cells. Irradiation inhibits these and all other cells having cell division activity. This experiment further demonstrates that the molecularly and microscopically detectable skeletal muscle cell precursor cells (FIG. 11A-B) are functional. In comparison, FIG. 11E then also shows muscle reconstruction in non-irradiated ESM compared with irradiated ESM by fluorescence microscopy. The detection of cells with sarcomeric actinin at day 21 after CTX-induced muscle cell destruction demonstrates muscle reconstruction in ESM via activation with cell division and differentiation of skeletal muscle cell progenitor cells. No regenerative activity could be detected in irradiated ESM. These morphological observations are consistent with the functional observations in FIG. 11D. These show that engineered skeletal muscles contract again with approximately 1 mN 21 days after CTX-mediated muscle cell destruction.

Methods for Examples 4 and 5

Maturation Conditions

The maturation medium was changed every other day and cultured under mechanical stretching for up to 9 weeks. The maturation medium comprised DMEM, with low glucose, GlutaMAX™ Supplement, pyruvate (Thermo Fisher Scientific), 1% N-2 Supplement (Thermo Fisher Scientific), 2% B-27 Supplement (Thermo Fisher Scientific), and optional antibiotics (e.g., 1% Pen/Strep-Thermo Fisher Scientific). 0.1 μM T3 (Sigma-Aldrich) or 1 mM creatine monohydrate (Sigma-Aldrich) were added to the maturation medium for a four-week period when indicated (e.g., day 28-56 or day 56-84).

Isometric Force Measurements

The contractile function of engineered skeletal muscle tissue was measured under isometric conditions in an organ bath filled with gassed (5% CO2/95% 02) Tyrode solution (containing in mmol/L): 120 NaCl, 1 MgCl2, 0.2 CaCl2), 5.4 KCl, 22.6 NaHCO₃, 4.2 NaH2PO4, 5.6 glucose, and 0.56 ascorbate) at 37° C. To verify the force-length relationship—while ESMs were electrically stimulated at 1 Hz with 5 ms rectangular pulses of 200 mA—muscle length was increased by mechanical stretching in intervals of 125 μm, until the maximum contraction force was observed. At the length of maximum force generation, tetanic contraction force was assessed under defined stimulation frequencies (4-second stimulation at 10, 20, 40, 60, 80, and 100 Hz).

Cardiotoxin Injury Model

The engineered skeletal muscles of the control were subjected to cardiotoxin injury (CTX) in parallel with the irradiated ESM. To induce injury, the tissue was maintained in maturation medium containing 25 μg/ml CTX (Latoxan) for 24 hours (Tiburcy et al., 2019). The injured tissue was then rinsed and placed in expansion medium consisting of DMEM, low glucose, GlutaMAX™ Supplement, pyruvate (Thermo Fisher Scientific), 1% N-2 Supplement (Thermo Fisher Scientific), 1% MEM non-essential amino acid solution (Thermo Fisher Scientific), 10 ng/ml HGF (Peprotech), and 10% knockout serum replacement (Thermo Fisher Scientific) for 1 week, and then cultured in maturation medium consisting of DMEM, low glucose, GlutaMAX™ supplement, pyruvate (Thermo Fisher Scientific), 1% N-2 Supplement (Thermo Fisher Scientific), 2% B-27 Supplement (Thermo Fisher Scientific), and 1 mM creatine monohydrate (Sigma-Aldrich) for an additional 2 weeks of regeneration. The medium was refreshed every other day. Optionally, antibiotics (e.g., 1% Pen/Strep-Thermo Fisher Scientific) may be added.

ESM Irradiation

ESM were placed in the culture dish in an STS Biobeam 8000 gamma irradiator 24 hours prior to CTX treatment, and exposed to a single dose of 30 Gy irradiation for 10 minutes (Tiburcy et al., 2019).

Immunostaining and Confocal Imaging.

2D cell cultures were fixed in formaldehyde 4% (Carl Roth) in phosphate-buffered saline (PBS) at room temperature for 15 min. Engineered skeletal muscles were fixed in 4% paraformaldehyde in PBS at 4° C. overnight. After fixation, engineered skeletal muscles were immersed in 70% ethanol (Carl Roth) for 1 min and then embedded in 2% agarose (peqGOLD) in 1× Tris acetate-EDTA (TAE) buffer. Sections were cut at 400 μm using a Leica Vibrotome (LEICAVT1000S) and stored in cold 1×PBS. Prior to staining, both 2D cell cultures and ESM sections were washed with 1×PBS. To induce blocking and permeabilization, samples were incubated in blocking buffer (1×PBS containing 5% fetal bovine serum, 1% bovine serum albumin (BSA), and 0.5% Triton-X). All primary and secondary antibody stainings were performed in the same blocking solution. The following antibodies were used for primary staining in RT for 4 h or at 4° C. for 24-72 h: Pax3 (1:100, DSHB), Pax7 (1:100, DSHB), MyoD (1:100, Dako), and myogenin (1:10, DSHB). Sarcomeric α-actinin (1:500, Sigma-Aldrich), laminin (1:50, Sigma-Aldrich). After 3×PBS washing, the appropriate Alexa fluorochrome-labelled secondary antibodies (1:1000, Thermo Fisher Scientific) were applied at room temperature for 2 h. In parallel with the secondary antibodies, Alexa 633-conjugated phalloidin (1:100, Thermo Fisher Scientific) and Hoechst 33342 (1:1000, Molecular Probes) were used for f-actin and nuclear staining, respectively. After 3 washes with PBS, samples were stained in Fluoromount-G (Southern Biotech). All images were acquired using a Zeiss LSM 710/NLO confocal microscope. To quantify the labelled cells, 3 random focal planes per sample from 3 different experiments were selected for analysis using the ImageJ Cell Counter Tool.

Western Blot Analysis

For protein isolation, engineered skeletal muscle was placed in an Eppendorf tube and snap frozen in liquid nitrogen. To the engineered skeletal muscle, 150 μl of ice-cold protein lysate buffer (2.38 g HEPES, 10.20 g NaCl, 100 ml glycerol, 102 mg MgCl2, 93 mg EDTA, 19 mg EGTA, 5 ml NP-40 in a total volume of 500 ml ddH2O) containing 1/10 phosphatase inhibitor (Roche) and 1/7 protease inhibitor (Roche) were added. A 7-mm stainless steel ball (Qiagen) was added to the Eppendorf tube, and the sample was homogenized using the TissueLyser II (Qiagen) for 30 seconds at 30 Hz and 4° C., followed by incubation on ice for 2 hours and then centrifugation at 12,000 rpm and 4° C. for 30 minutes. The supernatant was collected as a protein sample, and the protein concentration was measured by Bradford protein assay. 30 μg of the protein sample were loaded onto a 4 to 15% sodium dodecyl sulfate (SDS)-polyacrylamide gel (Bio-Rad), electrophoretically separated at 100 V for approximately 2.5 hours, and then transferred onto a polyvinylidene fluoride (PVDF) membrane at 30 V in an ice-filled box placed in cold storage overnight. To visualize the total protein, the PVDF membrane was stained with Ponceau Red. Staining with primary antibodies (4 hr in room temperature) and secondary antibodies (1 hr in room temperature) was performed in a blocking solution containing 5% milk in 1× Tris-buffered saline (TBS) and 0.1% Tween 20. Protein expression in ESM was analyzed by Western blot using the following primary antibodies: monoclonal embryonic myosin heavy chain 3 (1:500, F1.652, DSHB), slow myosin type heavy chain 7 (1:500, A4.951, DSHB), and fast myosin type heavy chain 2 (1:100, A4.74, DSHB). Protein loading was controlled by vinculin (VCL) antibody (1:5000, V3131, Sigma-Aldrich). The membrane was washed with 1× Tris-buffered saline (TBS) and 0.1% Tween 20 for 5 minutes. Horseradish peroxidase-conjugated goat anti-mouse IgG antibody (1:10,000, P0260, Dako) was used for the secondary staining. After washing the membrane with 1× Tris-buffered saline (TBS) and 0.1% Tween 20 for 5 min, the blot was covered with Femto LUCENTTmLuminol reagent (Gbiosciences), and protein bands were imaged using the BIO-RAD ChemiDocTMMP system. Protein quantification from the Western blot was performed using ImageJ.

Quantitative Real-Time PCR

Total RNA was isolated from 2D cell cultures and engineered skeletal muscle using Trizol reagent (Thermo Fisher Scientific). Trizol was added to the 2D cells in the culture plate, the cells were scraped off, and the cell lysate was homogenized by vortexing. The engineered skeletal muscle was placed in a polypropylene tube (Eppendorf) and snap frozen in liquid nitrogen. 1 ml of Trizol were added to the engineered skeletal muscle in the presence of a 7-mm stainless steel ball (Qiagen), and the sample was lysed using the TissueLyser II (Qiagen) for 2 min at 30 Hz and 4° C. RNA isolation was performed according to the manufacturer's protocol. The RNA concentration was quantified using the Nanodrop spectrophotometer (Thermo Fisher Scientific). According to the manufacturer's instructions, 1 μg of the RNA sample was treated with DNase I (Roche), and then the sample was reverse transcribed into complementary DNA (cDNA) using the High Capacity cDNA Reverse Transcription Kit (Applied Biosystems). Quantitative PCR was performed using the Fast SYBR Green Master Mix (Thermo Fisher Scientific) and the AB7900 HT Fast Real-Time PCR System (Applied Biosystems). Alternatively, transcriptome analysis was performed by RNA sequencing using an Illumina platform.

Materials Used in all Examples

The materials used herein are commercially available unless otherwise noted. For example, penicillin/streptomycin, B27 serum-free additive, essential amino acids (MEM-NEAA), and 2-mercaptoethanol are available from Invitrogen. The name of the company is indicated with each of the materials used.

The stock solutions of N2 and B27 serum-free additive solutions were stored at −20° C. Once thawed, they were added to the medium and stored at 4° C. for a maximum of one week. Knockout serum replacement stock solutions were also stored at −20° C. Once thawed, they were stored at 4° C. for a maximum of two weeks. The LDN193189 stock solution had a concentration of 10 mM in DMSO and was stored at −20° C. The DAPT stock solution had a concentration of 20 mM in DMSO and was stored at −20° C. The bFGF stock solution had a concentration of 10 μg/ml in PBS containing 0.1% human recombinant albumin and was stored at −20° C. The HGF stock solution had a concentration of 10 μg/ml in PBS containing 0.1% human recombinant albumin and was stored at −20° C. The Rock inhibitor had a concentration of 10 mM in DMSO and was stored at −20° C.

Once the stock solutions of growth factors and small molecules were thawed, they were stored at 4° C. for a maximum of one week.

TABLE 1
Composition of the serum-free additive N-2 in 100x effective
concentration (liquid form), i.e. 1% (v/v) corresponds
to a single (1x) effective concentration

| | Molecular | Concentration | Concentration |
|---|---|---|---|
| Components | weight | (μg/ml) | (mM) |
| Human transferrin (Holo) | 10000.0 | 10000.0 | 1.0 |
| Insulin, recombinant full | 5807.7 | 500.0 | 0.0860926 |
| chain | | | |
| Progesterone | 314.47 | 0.63 | 0.0020033708 |
| Putrescine | 161.0 | 1611.0 | 10.006211 |
| Selenite | 173.0 | 0.52 | 0.0030057803 |

TABLE 2
Composition of the non-essential amino acids
in 100x effective concentration (100x)

| | Molecular | Concentration | Concentration |
|---|---|---|---|
| Components | weight | (mg/L) | (mM) |
| Glycine | 75.0 | 750.0 | 10.0 |
| L-Alanine | 89.0 | 890.0 | 10.0 |
| L-Asparagine | 132.0 | 1320.0 | 10.0 |
| L-Aspartic acid | 133.0 | 1330.0 | 10.0 |
| L-Glutamic acid | 147.0 | 1470.0 | 10.0 |
| L-Proline | 115.0 | 1150.0 | 10.0 |
| L-Serine | 105.0 | 1050.0 | 10.0 |

TABLE 3
DMEM, low glucose at 1 g/l, GlutaMAX ™ supplemented
with pyruvate (Gibco, catalogue number: 10567014)

| | | | |
|---|---|---|---|
| | Molecular | Concentration | Concentration |
| Components | weight | (mg/L) | (mM) |

Amino acids

| | | | |
|---|---|---|---|
| Glycine | 75.0 | 30.0 | 0.4 |
| L-Alanyl-Glutamine | 217.0 | 862.0 | 3.9723501 |
| L-Arginine-Hydrochloride | 211.0 | 84.0 | 0.39810428 |
| L-Cystine | 313.0 | 48.0 | 0.15335463 |
| L-Histidine-Hydrochloride-H₂O | 210.0 | 42.0 | 0.2 |
| L-Isoleucine | 131.0 | 105.0 | 0.8015267 |
| L-Leucine | 131.0 | 105.0 | 0.8015267 |
| L-Lysine-Hydrochloride | 183.0 | 146.0 | 0.7978142 |
| L-Methionine | 149.0 | 30.0 | 0.20134228 |
| L-Phenylalanine | 165.0 | 66.0 | 0.4 |
| L-Serine | 105.0 | 42.0 | 0.4 |
| L-Threonin | 119.0 | 95.0 | 0.79831934 |
| L-Tryptophane | 204.0 | 16.0 | 0.078431375 |
| L-Tyrosine | 181.0 | 72.0 | 0.39779004 |
| L-Valine | 117.0 | 94.0 | 0.8034188 |

Vitamins

| | | | |
|---|---|---|---|
| Choline chloride | 140.0 | 4.0 | 0.028571429 |
| D-Calcium pantothenate | 477.0 | 4.0 | 0.008385744 |
| Folic acid | 441.0 | 4.0 | 0.009070295 |
| Niacinamide | 122.0 | 4.0 | 0.032786883 |
| Pyridoxine hydrochloride | 206.0 | 4.0 | 0.019417476 |
| Riboflavin | 376.0 | 0.4 | 0.0010638298 |
| Thiamine hydrochloride | 337.0 | 4.0 | 0.011869436 |
| i-Inositol | 180.0 | 7.2 | 0.04 |

Inorganic salts

| | | | |
|---|---|---|---|
| Calcium chloride (CaCl₂•2H₂O) | 147.0 | 264.0 | 1.7959183 |
| Iron-III-Nitrate (Fe(NO₃)₃•9H₂O) | 404.0 | 0.1 | 2.4752476E−4 |
| Magnesium sulfate (MgSO₄—7H₂O) | 246.0 | 200.0 | 0.8130081 |
| Potassium chloride (KCl) | 75.0 | 400.0 | 5.3333335 |
| Sodium bicarbonate (NaHCO₃) | 84.0 | 3700.0 | 44.04762 |
| Sodium chloride (NaCl) | 58.0 | 6400.0 | 110.344826 |
| Sodium phosphate monobasic | 156.0 | 141.0 | 0.90384614 |
| (NaH₂PO₄—2H₂O) | | | |

Other Components

| | | | |
|---|---|---|---|
| D-Glucose (Dextrose) | 180.0 | 1000.0 | 5.5555553 |
| Phenol red | 376.4 | 15.0 | 0.039851222 |
| Sodium pyruvate | 110.0 | 110.0 | 1.0 |

TABLE 4
Composition of the additional serum-free B27
additive at 50X effective concentration (liquid form)
10 ml of the 50X B27 additive per 500
ml of medium corresponds to 2% (v/v)

| | Concentration in 50x B27 |
|---|---|
| Components | μg/ml |
| BSA fraction V IgG free Fatty Acid Poor | 125000 |
| Catalase | 125 |
| Glutathion reduced | 50 |
| Humane insulin | 156.25 |
| Superoxid dismutase | 125 |
| Humane Holo-Transferin | 250 |
| T3 | 0.1 |
| L-carnitine | 100 |
| Ethanolamine | 50 |
| D+-galactose | 750 |
| Putrescine | 805 |
| Sodium selenite | 0.625 |
| Corticosterone | 1 |
| Linoleic acid | 50 |
| Linolenic acid | 50 |
| Progesterone | 0.315 |
| Retinyl acetate | 5 |
| DL -alpha tocopherol (Vit E) | 50 |
| DL-alpha tocopherol acetate | 50 |
| Biotin | 125 |

TABLE 5
Composition of Knockout Serum Replacement (KSR)

| | Concentration in the | | Concentration in the |
|---|---|---|---|
| Substance | additive (μg/ml) | Substance | additive (ug/ml) |
| Glycine | 150 | Ag⁺ | 0.0006 |
| L-Histidine | 940 | Al³⁺ | 0.0007 |
| L-Isoleucine | 3400 | Ba²⁺ | 0.008 |
| L-Methionine | 90 | Cd²⁺ | 0.03 |
| L-Phenylalanine | 1800 | Cr³⁺ | 0.003 |
| L-Proline | 4000 | Ge⁴⁺ | 0.003 |
| L-Hydroxyproline | 100 | Se⁴⁺ | 0.02 |
| L-Serine | 800 | Br⁻ | 0.004 |
| L-Threonine | 2200 | I⁻ | 0.0007 |
| L-Tryptophane | 440 | Mn²⁺ | 0.0004 |
| L-Tyrosine | 77 | F⁻ | 0.010 |
| L-Valine | 2400 | Si⁴⁺ | 0.01 |
| Thiamine | 33 | V⁵⁺ | 0.003 |
| Reduced glutathione | 10 | Mp⁶⁺ | 0.006 |
| Ascorbic acid -2-PO₄ (Mg salt) | 330 | Ni²⁺ | 0.0002 |
| Transferrin | 55 | Rb⁺ | 0.005 |
| Insulin | 100 | Sn²⁺ | 0.0002 |
| Sodium selenite | 0.07 | Zr⁴⁺ | 0.01 |
| AlbuMAX | 83 000 | | |
| AgNO₃ | 0.0009 | KBr | 0.0006 |
| AlCl₃ 6H₂O | 0.006 | KI | 0.0009 |
| Ba(C₂H₃O₂)₂ | 0.01 | MnCl₂ 4H₂O | 0.002 |
| CdSO₄ 8H₂O | 0.08 | NaF | 0.02 |
| CoCl₂ 6H₂O | 0.01 | Na₂SiO₃ 9H₂O | 1 |
| Cr₂(SO₄)₃ 1H₂O | 0.003 | NaVO₃ | 0.006 |
| GeO₂ | 0.003 | (NH₄)₆Mo₇O₂₄ 4H₂O | 0.06 |
| Na₂SeP₃ | 0.007 | RbCl | 0.007 |
| H₂SeO₃ | 0.02 | SnCl₂ | 0.0003 |
| | | ZrOCl₂ 8H₂O | 0.02 |

### REFERENCES

WO 98/30679
WO 2017/100498 A1
WO 2018/170180 A1
Baghbaderani B A, Tian X, Neo B H, Burkall A, Dimezzo T, Sierra G, Zeng X, Warren K, Kovarcik D P, Fellner T, Rao M S. cGMP-Manufactured Human Induced Pluripotent Stem Cells Are Available for Pre-clinical and Clinical Applications. Stem Cell Reports. 5(4):647-59.
Beldjilali-Labro M, Garcia Garcia A, Farhat F, Bedoui F, Grosset J, Dufresne M, Legallais C. Biomaterials in Tendon and Skeletal Muscle Tissue Engineering: Current Trends and Challenges. Materials 2018 (11) 1116
Brewer, G. J., Torricelli, J. R., Evege, E. K. and Price, P. J. (1993), Optimized survival of hippocampal neurons in B27-supplemented neurobasal™, a new serum-free medium combination. J. Neurosci. Res., 35: 567-576. doi:10.1002/jnr.490350513
Chal J et al. “Generation of human muscle fibers and satellite-like cells from human pluripotent stem cells in vitro”, Nature Protocols, Vol. 11 No. 10, 2016 S.1833-1846
Espejel S, Eckardt S, Harbell J, Roll G R, McLaughlin K J, Willenbring H. Brief report: Parthenogenetic embryonic stem cells are an effective cell source for therapeutic liver repopulation. Stem Cells. 2014 July;32(7):1983-8. doi: 10.1002/stem.1726.
Hughes, C. S., Postovit, L. M. and Lajoie, G. A. (2010), Matrigel: A complex protein mixture required for optimal growth of cell culture. Proteomics, 10: 1886-1890. doi:10.1002/pmic.200900758
James E. Hudson, Gary Brooke, Chris Blair, Ernst Wolvetang, and Justin John Cooper-White. Tissue Engineering Part A. September 2011
Khodabukus A, Prabhu N, Wang J, Bursac N. In Vitro Tissue-Engineered Skeletal Muscle Models for Studying Muscle Physiology and Disease. Adv. Healthcare Mater. 2018 (7) 1701498
Krämer et al. “Tissue engineered muscle to investigate skeletal muscle regeneration in vitro”, Naunyn-Schmiedeberg's Archieves of Pharmacology, (February 2014) Vol. 387, Supplement 1 S. S59, Abstract Number: 235
Liao I C, Liu J B, Bursac N, Leong K W. Effect of Electromechanical Stimulation on the Maturation of Myotubes on Aligned Electrospun Fibers. Cell Mol Bioeng. 2008 Sep. 1; 1(2-3):133-145.
Long C, Li H, Tiburcy M, Rodriguez-Caycedo C, Kyrychenko V, Zhou H, Zhang Y, Min Y L, Shelton J M, Mammen P P A, Liaw N Y, Zimmermann W H, Bassel-Duby R, Schneider J W, Olson E N. Correction of diverse muscular dystrophy mutations in human engineered heart muscle by single-site genome editing. Sci Adv. 2018 Jan. 31; 4(1):eaap9004.
Pavesi A, Adriani G, Rasponi M, Zervantonakis I K, Fiore G B, Kamm R D. Controlled electromechanical cell stimulation on-a-chip. Sci Rep. 2015 Jul. 2; 5:11800.
Rao L, Qian Y, Khodabukus A, Ribar T, Bursac N. Engineering human pluripotent stem cells into a functional skeletal muscle tissue. Nature Communications. 2018; 9(126)
Shahriyari et al. “Engineered human skeletal muscle from pluripotent stem cell-derived myoblasts: a novel pharmaceutical tool”, Naunyn-Schmiedeberg's Archives of Pharmacology, (February 2018) Vol. 391, Supp. Supplement 1, S. S8, Abstract Number: 25.
Takahashi, Kazutoshi et al. Induction of Pluripotent Stem Cells from Adult Human Fibroblasts by Defined Factors. Cell. 2007; 131(5), 861-872
Takahashi K, Yamanaka S. Induction of Pluripotent Stem Cells from Mouse Embryonic and Adult Fibroblast Cultures by Defined Factors. Cell. 2006 Aug. 25; 126(4):P663-676
Tedesco F S, Dellavalle A, Diaz-Manera J, Messina G, Cossu G. Repairing skeletal muscle: regenerative potential of skeletal muscle stem cells. J Clin Invest. 2010; 120(1):11-19.
Thomson J, Itskovitz-Eldor, Shapiro et al. Embryonic Stem Cell Lines Derived from Human Blastocysts. Science. 6 Nov. 1998. 282(5391), 1145-1147
Tiburcy M, Hudson J E, Balfanz P, Schlick S, Meyer T, Chang Liao M L, Levent E, Raad F, Zeidler S, Wingender E, Riegler J, Wang M, Gold J D, Kehat I, Wettwer E, Ravens U, Dierickx P, van Laake L W, Goumans M J, Khadjeh S, Toischer K, Hasenfuss G, Couture L A, Unger A, Linke W A, Araki T, Neel B, Keller G, Gepstein L, Wu J C, Zimmermann W H. Defined Engineered Human Myocardium With Advanced Maturation for Applications in Heart Failure Modeling and Repair. Circulation. 2017 May 9;135(19): 1832-1847.
Tiburcy, M, Markov, A, Kraemer, L K, et al. Regeneration competent satellite cell niches in rat engineered skeletal muscle. FASEB BioAdvances. 2019; 1: 731-746.
Yin H, Price F, Rudnicki M A. Satellite cells and the muscle stem cell niche. Physiol Rev. 2013; 93(1):23-67.
Zimmermann W H, Melnychenko I, Eschenhagen T. Engineered Heart Tissue for Regeneration of Diseased Hearts. Biomaterials (2004) 25:1639-1647

1.-96. (canceled)

97. A method for producing engineered skeletal muscle tissue from pluripotent stem cells, comprising the steps of
(i) inducing mesoderm differentiation of the pluripotent stem cells by culturing pluripotent stem cells in a basal medium comprising an effective amount of (a) FGF2, (b) a GSK3 inhibitor, (c) a SMAD inhibitor, and (d) a serum-free additive comprising transferrin, insulin, progesterone, putrescine and selenium or a bioavailable salt thereof;
(ii) inducing myogenic specification by culturing the cells obtained in step (i) in a basal medium comprising an effective amount of (a) a gamma-secretase/NOTCH inhibitor, (b) FGF2, and (c) a serum-free additive as in (i), followed by continuing the cultivation in the medium with the addition of an effective amount of (d) HGF, followed by culturing the cells in a basal medium comprising an effective amount of (a) a gamma secretase/NOTCH inhibitor, (b) HGF, (c) a serum-free additive as in (i), and (d) knockout serum replacement (KSR);
(iii) expanding and maturing the cells into skeletal myoblasts and satellite cells by culturing the cells obtained in step (ii) in a basal medium comprising an effective amount of (a) HGF, (b) a serum-free additive as in (i), and (c) knockout serum replacement (KSR);
(iv) maturing the cells into skeletal myotubes and satellite cells by culturing the cells obtained in step (iii), which are dispersed in an extracellular matrix, under mechanical stimulation in a basal medium, comprising an effective amount of (a) a serum-free additive as in step (i), and (b) an additional serum-free additive comprising albumin, transferrin, ethanolamine, selenium or a bioavailable salt thereof, L-carnitine, fatty acid additive, and triiodo-L-thyronine (T3);
thereby producing engineered skeletal muscle tissue.

98. The method of claim 97, wherein the skeletal muscle tissue generates a contraction force of at least 0.6 millinewtons (mN) upon a stimulus of 100 Hz, preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, more preferably at least 2 mN, more preferably at least 2.3 mN, more preferably at least 2.6 mN, even more preferably at least 3 mM, even more preferably at least 3.3 mN, even more preferably at least 3.6 mN, and most preferably at least 4 mN.

99. The method of claim 97, wherein in step (iv), the mechanical stimulation is a static tension, a dynamic stimulation, or an auxotonic stimulation, preferably wherein the mechanical stimulation is a static tension.

100. The method claim 97, comprising, prior to step (i), a seeding step, wherein the pluripotent stem cells are seeded in a stem cell medium in the presence of a ROCK inhibitor, and preferably wherein the pluripotent stem cells in the seeding step are first seeded into an engineered form in the presence of one or more components of an extracellular matrix in a master mix before the stem cell medium is added.

101. The method of claim 97, wherein after step (iii), the skeletal myoblasts and satellite cells are seeded into an engineered form in an additional step prior to step (iv) in the presence of one or more components of an extracellular matrix in a master mix.

102. The method of claim 100, wherein the engineered form has the form of a ring, ribbon, strand, patch, pouch, or cylinder, wherein optionally individual skeletal muscle tissues are fused.

103. The method of claim 97, wherein the method does not comprise a differentiation- or maturation-related transgene, preferably wherein the method does not comprise a myogenic transgene, more preferably wherein the method does not comprise the transgene Pax7 or MyoD; and/or
wherein the method does not comprise a skeletal myoblast enrichment step, preferably not an enrichment step by cell selection, more preferably not an enrichment step by antibody-based cell selection.

104. The method of claim 97, wherein the basal medium in step (iv) comprises an effective amount of creatine and/or triiodo-L-thyronine (T3).

105. The method of claim 97, wherein the skeletal muscle tissue has a contraction speed of at least 3 mN/sec upon a stimulation of 100 Hz, preferably at least 4 mN/sec, more preferably at least 5 mN/sec, more preferably at least 6 mN/sec, even more preferably at least 6.5 mN/sec, even more preferably at least 7 mN/sec; and/or
wherein the skeletal muscle tissue has a relaxation speed of at least 0.5 mN/sec upon termination of a stimulation of 100 Hz, preferably at least 0.7 mN/sec, more preferably at least 0.9 mN/sec more preferably at least 1 mN/sec, even more preferably at least 1.2 mN/sec, even more preferably at least 1.5 mN/sec.

106. An engineered skeletal muscle tissue, having multinuclear mature skeletal muscle fibers with satellite cells, and having no blood supply and/or no central nervous system control, wherein the skeletal muscle tissue is serum-free and/or does not comprise a differentiation- or maturation-related transgene, preferably wherein the skeletal muscle tissue does not comprise a myogenic transgene, more preferably wherein the skeletal muscle tissue does not comprise the Pax7 or MyoD transgene.

107. The engineered skeletal muscle tissue of claim 106, wherein the skeletal muscle tissue generates at least a contraction force of 0.6 millinewtons (mN) upon a stimulus of 100 Hz, preferably at least 0.7 mN, more preferably at least 0.8 mN, more preferably at least 0.9 mN, more preferably at least 1 mN, more preferably at least 1.2 mN, more preferably at least 1.3 mN, more preferably at least 1.4 mN, more preferably at least 1.5 mN, more preferably at least 1.6 mN, more preferably at least 1.7 mN, more preferably at least 1.8 mN, more preferably at least 1.9 mN, more preferably at least 2 mN, more preferably at least 2.3 mN, more preferably at least 2.6 mN, even more preferably at least 3 mM, even more preferably at least 3.3 mN, even more preferably at least 3.6 mN, and most preferably at least 4 mN; and or
wherein the skeletal muscle tissue has a contraction speed of at least 3 mN/sec upon a stimulation of 100 Hz, preferably at least 4 mN/sec, more preferably at least 5 mN/sec, more preferably at least 6 mN/sec, even more preferably at least 6.5 mN/sec, even more preferably at least 7 mN/sec; and/or
wherein the skeletal muscle tissue has a relaxation speed of at least 0.5 mN/sec upon termination of a stimulation of 100 Hz, preferably at least 0.7 mN/sec, more preferably at least 0.9 mN/sec, more preferably at least 1 mN/sec, even more preferably at least 1.2 mN/sec, even more preferably at least 1.5 mN/sec.

108. A skeletal muscle tissue according to claim 106 for use in medicine.

109. An in vitro method for testing the efficacy of a drug candidate on a skeletal muscle tissue, comprising the steps of
(a) providing a skeletal muscle tissue according to claim 106,
(b) optionally inflicting damage on the skeletal muscle tissue, and
(c) contacting the skeletal muscle tissue of step (a) or (b) with a drug candidate;
preferably wherein the method further comprises determining the contraction force and/or the structure of the skeletal muscle tissue and/or the metabolic function and/or molecular parameters and/or protein biochemical parameters before and/or after step (c).

110. An in vitro method for testing the toxicity of a substance on a skeletal muscle tissue, comprising the steps of
(a) providing a skeletal muscle tissue according to claim 106,
(b) contacting the skeletal muscle tissue from step (a) with a substance to be tested,
preferably wherein the method further comprises determining the contraction force and/or skeletal muscle tissue structure and/or metabolic function and/or molecular parameters and/or protein biochemical parameters before and/or after step (b).

111. An in vitro method for testing the effect of nutrients and dietary supplements on skeletal muscle tissue performance, comprising the steps of
(a) providing a skeletal muscle tissue according to claim 106,
(b) contacting the skeletal muscle tissue from step (a) with a nutrient or dietary supplement to be tested
preferably wherein the method further comprises determining the contraction force and/or the structure of the skeletal muscle tissue and/or the metabolic function and/or molecular parameters and/or protein biochemical parameters before and/or after step (b).

112. The method of claim 101, wherein the engineered form has the form of a ring, ribbon, strand, patch, pouch, or cylinder, wherein optionally individual skeletal muscle tissues are fused.
